SEQUENCE LISTING (158 sequences)

SEQ ID NO: 1 - protein, 113 aa, Artificial Sequence
  Description of Artificial Sequence Synthetic polypeptide
MGGLEPCSRLLLLPLLLAVSGLRPVQAQAQSDCSCSTVSPGVLAGIVMGDLVLTVLIALAVYFLGRLVPRGRGAAEAATRKQRITETESPYQELQGQRSDVYSDLNTQRPYYK

SEQ ID NO: 2 - DNA, 342 bp, Artificial Sequence
  Description of Artificial Sequence Synthetic polynucleotide
atggggggacttgaaccctgcagcaggctcctgctcctgcctctcctgctggctgtaagtggtctccgtcctgtccaggcccaggcccagagcgattgcagttgctctacggtgagcccgggcgtgctggcagggatcgtgatgggagacctggtgctgacagtgctcattgccctggccgtgtacttcctgggccggctggtccctcgggggcgaggggctgcggaggcagcgacccggaaacagcgtatcactgagaccgagtcgccttatcaggagctccagggtcagaggtcggatgtctacagcgacctcaacacacagaggccgtattacaaatga

SEQ ID NO: 3 - protein, 67 aa, Artificial Sequence
  Description of Artificial Sequence Synthetic polypeptide
VLVVVGGVLACYSLLVTVAFIIFWVRSKRSRLLHSDYMNMTPRRPGPTRKHYQPYAPPRDFAAYRSR

SEQ ID NO: 4 - DNA, 196 bp, Artificial Sequence
  Description of Artificial Sequence Synthetic polynucleotide
gtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgct

SEQ ID NO: 5 - protein, 247 aa, Artificial Sequence
  Description of Artificial Sequence Synthetic polypeptide
SYVTVSSQDPAEPKSPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPK

SEQ ID NO: 6 - DNA, 734 bp, Artificial Sequence
  Description of Artificial Sequence Synthetic polynucleotide
gtacgtcactgtctcttcacaggatcccgccgagcccaaatctcctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcaaccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaaaagatc

SEQ ID NO: 7 - DNA, 660 bp, Artificial Sequence
  Description of Artificial Sequence Synthetic polynucleotide
atgagcactgaaatgcatcccggaagggggtcctggcacgaggaggcgctccccaagaagacaggggggccccagggctccaggcggtgcttgttcctcagcctcttctccttcctgatcgtggcaggcgccaccacgctcttcttcctgctgcactttggagtgatcggcccccagagggaagagttccccagggacctctctctaatcagccctctgcaggcagcccatgttgtagcaaaccctcaagctgaggggcagctccagtggctgaaccgccgggccaatgccctcctggccaatggcgtggagctgagagataaccagctggttgtgccatcagagggcctgtacctcatctactcccaggtcctcttcaagggccaaggctgcccctccacccatgtgctcctcacccacaccatcagccgcatcgccgtctcccaccagaccaaggtcaacctcctcttcgccatcaagagcccctgccagagggagaccccagagggggctgaggctaagccctggtatgagcccatctatctgggaggggtcttccagctggagaagggtgaccgactcatcgctgagatcaatcggcccgactatctctactttgccgagtatgggcaggtctactttgggatcattgccctgtcg

SEQ ID NO: 8 - DNA, 813 bp, Artificial Sequence
  Description of Artificial Sequence Synthetic polynucleotide
atgcttctcctggtgacaagccttctgctctgtgagttaccacacccagcattcctcctgatcccaggggacattgttttgacccaatcacctccctctctcgccatgtccttgggtaaacgggcaacaatctcctgtagagcttccgaaagtgtaacaattcttggaagccacctcatacattggtatcagcaaaagccggggcagccccctacattgctcattcagttggcttcaaatgtccagacgggtgtaccagcgagattctcagggagtggctcccgaacggatttcacactgacgattgatcccgtcgaagaggacgatgtcgcagtttattattgcctccaaagtcggacaattccgaggacttttggaggcggaacaaaattggaaatcaaagggggtggaggttctggcggagggggcagcggtggtggaggaagtgggggcggtgggagtcaaatccagctcgtccaatccggtccagagttgaagaaacccggcgagacggtaaaaatcagctgtaaagcctcaggttacacgtttacggactatagcattaattgggttaagagggctccggggaaggggctcaaatggatgggctggataaacacagagacgagagagcccgcatatgcgttcgactttagaggtcgattcgctttcagtcttgaaacctctgcttctaccgcgtatctccagataaacaacctgaaatatgaggatacagcaacttatttttgcgctctcgattacagctatgcgatggattattggggacaaggaacttccgtgactgtgtcaagc

SEQ ID NO: 9 - protein, 270 aa, Artificial Sequence
  Description of Artificial Sequence Synthetic polypeptide
MLLLVTSLLLCELPHPAFLLIPDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGGGGSGGGGSGGGGSGGGGSQIQLVQSGPELKKPGETVKISCKASGYTFTDYSINWVKRAPGKGLKWMGWINTETREPAYAFDFRGRFAFSLETSASTAYLQINNLKYEDTATYFCALDYSYAMDYWGQGTSVTVSS

SEQ ID NO: 10 - DNA, 812 bp, Artificial Sequence
  Description of Artificial Sequence Synthetic polynucleotide
tgcttctcctggtgacaagccttctgctctgtgagttaccacacccagcattcctcctgatcccaggggacatcgtgctgacccagagcccccccagcctggccatgtctctgggcaagagagccaccatcagctgccgggccagcgagagcgtgaccatcctgggcagccacctgatctactggtatcagcagaagcctggccagccccccaccctgctgatccagctggctagcaatgtgcagaccggcgtgcccgccagattcagcggcagcggcagcagaaccgacttcaccctgaccatcgaccccgtggaagaggacgacgtggccgtgtactactgcctgcagagccggaccatcccccggacctttggcggaggaacaaagctggaaatcaagggtggtggtggttctggtggtggtggttctggcggcggcggctccggtggtggtggatcccagattcagctggtgcagagcggccctgagctgaagaaacccggcgagacagtgaagatcagctgcaaggcctccggctacaccttccggcactacagcatgaactgggtgaaacaggcccctggcaagggcctgaagtggatgggccggatcaacaccgagagcggcgtgcccatctacgccgacgacttcaagggcagattcgccttcagcgtggaaaccagcgccagcaccgcctacctggtgatcaacaacctgaaggacgaggataccgccagctacttctgcagcaacgactacctgtacagcctggacttctggggccagggcaccgccctgaccgtgtccagc

SEQ ID NO: 11 - protein, 271 aa, Artificial Sequence
  Description of Artificial Sequence Synthetic polypeptide
MLLLVTSLLLCELPHPAFLLIPGDIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIKGGGGSGGGGSGGGGSGGGGSQIQLVQSGPELKKPGETVKISCKASGYTFRHYSMNWVKQAPGKGLKWMGRINTESGVPIYADDFKGRFAFSVETSASTAYLVINNLKDEDTASYFCSNDYLYSLDFWGQGTALTVSS

SEQ ID NO: 12 - DNA, 801 bp, Artificial Sequence
  Description of Artificial Sequence Synthetic polynucleotide
atggccctgcctgtgacagctctgctcctccctctggccctgctgctccatgccgccagacccgacatcgtgctgacccagagcccccccagcctggccatgtctctgggcaagagagccaccatcagctgccgggccagcgagagcgtgaccatcctgggcagccacctgatccactggtatcagcagaagcccggccagccccccaccctgctgatccagctcgccagcaatgtgcagaccggcgtgcccgccagattcagcggcagcggcagcagaaccgacttcaccctgaccatcgaccccgtggaagaggacgacgtggccgtgtactactgcctgcagagccggaccatcccccggacctttggcggaggcaccaaactggaaatcaagggcagcaccagcggctccggcaagcctggctctggcgagggcagcacaaagggacagattcagctggtgcagagcggccctgagctgaagaaacccggcgagacagtgaagatcagctgcaaggcctccggctacaccttcaccgactacagcatcaactgggtgaaaagagcccctggcaagggcctgaagtggatgggctggatcaacaccgagacaagagagcccgcctacgcctacgacttccggggcagattcgccttcagcctggaaaccagcgccagcaccgcctacctgcagatcaacaacctgaagtacgaggacaccgccacctacttttgcgccctggactacagctacgctatggactactggggccagggcaccagcgtgaccgtgtccagc

SEQ ID NO: 13 - protein, 267 aa, Artificial Sequence
  Description of Artificial Sequence Synthetic polypeptide
MALPVTALLLPLALLLHAARPDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 14 - DNA, 803 bp, Artificial Sequence
  Description of Artificial Sequence Synthetic polynucleotide
ggatggccctgcctgtgacagctctgctgctgcccctggccctgctgctccatgccgccagacccgacatcgtgctgacccagagcccccccagcctggccatgtctctgggcaagagagccaccatcagctgccgggccagcgagagcgtgaccatcctgggcagccacctgatctactggtatcagcagaagcctggccagccccccaccctgctgatccagctggctagcaatgtgcagaccggcgtgcccgccagattcagcggcagcggcagcagaaccgacttcaccctgaccatcgaccccgtggaagaggacgacgtggccgtgtactactgcctgcagagccggaccatcccccggacctttggcggaggaacaaagctggaaatcaagggcagcaccagcggctccggcaagcctggctctggcgagggcagcacaaagggacagattcagctggtgcagagcggccctgagctgaagaaacccggcgagacagtgaagatcagctgcaaggcctccggctacaccttccggcactacagcatgaactgggtgaaacaggcccctggcaagggcctgaagtggatgggccggatcaacaccgagagcggcgtgcccatctacgccgacgacttcaagggcagattcgccttcagcgtggaaaccagcgccagcaccgcctacctggtgatcaacaacctgaaggacgaggataccgccagctacttctgcagcaacgactacctgtacagcctggacttctggggccagggcaccgccctgaccgtgtccagc

SEQ ID NO: 15 - protein, 243 aa, Artificial Sequence
  Description of Artificial Sequence Synthetic polypeptide
DIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIKGGGGSGGGGSGGGGSQIQLVQSGPELKKPGETVKISCKASGYTFRHYSMNWVKQAPGKGLKWMGRINTESGVPIYADDFKGRFAFSVETSASTAYLVINNLKDEDTASYFCSNDYLYSLDFWGQGTALTVSS

SEQ ID NO: 16 - DNA, 810 bp, Artificial Sequence
  Description of Artificial Sequence Synthetic polynucleotide
atggagtttgggctgagctggctttttcttgtggctattttaaaaggtgtccagtgctctagacagatacagctcgtccaatccggtcccgatttgaaaaagcctggcgaaacagttaaactgtcatgtaaggcgagcggatacacgtttacgaacttcgggatgaattgggtaaaacaggccccgggaaaaggttttaagtggatggcttggataaacacctacactggtgagtcctacttcgcagacgatttcaaagggcggttcgcgttttcagtagagacttccgccacaactgcttatctccaaataaacaacttgaagaccgaggatacggcaacctacttttgcgctcggggcgagatttattatggatatgacggcgggttcgcttactggggtcaggggacgttggttaccgtgtctgccggtggtggtggttctggtggtggtggttctggcggcggcggctccggtggtggtggatccgacgtggtgatgacgcagagccaccgattcatgagtacctctgtaggcgaccgcgtctcaattacttgtcgagcgtctcaggacgtaaatacagcggtgagctggtatcagcaaaagcccggacagagcccgaaattgctgatcttttcagcctcatacagatataccggagtcccagaccgctttacaggttccggtagtggcgcggactttactctcacaatcagctctgtacaagctgaagatttggctgtttactattgtcagcagcactatagtacgccctggaccttcgggggcggtacgaagttggatattaag

SEQ ID NO: 17 - protein, 270 aa, Artificial Sequence
  Description of Artificial Sequence Synthetic polypeptide
MEFGLSWLFLVAILKGVQCSRQIQLVQSGPDLKKPGETVKLSCKASGYTFTNFGMNWVKQAPGKGFKWMAWINTYTGESYFADDFKGRFAFSVETSATTAYLQINNLKTEDTATYFCARGEIYYGYDGGFAYWGQGTLVTVSAGGGGSGGGGSGGGGSGGGGSDVVMTQSHRFMSTSVGDRVSITCRASQDVNTAVSWYQQKPGQSPKLLIFSASYRYTGVPDRFTGSGSGADFTLTISSVQAEDLAVYYCQQHYSTPWTFGGGTKLDIK

SEQ ID NO: 18 - DNA, 810 bp, Artificial Sequence
  Description of Artificial Sequence Synthetic polynucleotide
atggagtttgggctgagctggctttttcttgtggctattttaaaaggtgtccagtgctctagagacgtggtgatgacgcagagccaccgattcatgagtacctctgtaggcgaccgcgtctcaattacttgtcgagcgtctcaggacgtaaatacagcggtgagctggtatcagcaaaagcccggacagagcccgaaattgctgatcttttcagcctcatacagatataccggagtcccagaccgctttacaggttccggtagtggcgcggactttactctcacaatcagctctgtacaagctgaagatttggctgtttactattgtcagcagcactatagtacgccctggaccttcgggggcggtacgaagttggatattaagggtggtggtggttctggtggtggtggttctggcggcggcggctccggtggtggtggatcccagatacagctcgtccaatccggtcccgatttgaaaaagcctggcgaaacagttaaactgtcatgtaaggcgagcggatacacgtttacgaacttcgggatgaattgggtaaaacaggccccgggaaaaggttttaagtggatggcttggataaacacctacactggtgagtcctacttcgcagacgatttcaaagggcggttcgcgttttcagtagagacttccgccacaactgcttatctccaaataaacaacttgaagaccgaggatacggcaacctacttttgcgctcggggcgagatttattatggatatgacggcgggttcgcttactggggtcaggggacgttggttaccgtgtctgcc

SEQ ID NO: 19 - protein, 270 aa, Artificial Sequence
  Description of Artificial Sequence Synthetic polypeptide
MEFGLSWLFLVAILKGVQCSRDVVMTQSHRFMSTSVGDRVSITCRASQDVNTAVSWYQQKPGQSPKLLIFSASYRYTGVPDRFTGSGSGADFTLTISSVQAEDLAVYYCQQHYSTPWTFGGGTKLDIKGGGGSGGGGSGGGGSGGGGSQIQLVQSGPDLKKPGETVKLSCKASGYTFTNFGMNWVKQAPGKGFKWMAWINTYTGESYFADDFKGRFAFSVETSATTAYLQINNLKTEDTATYFCARGEIYYGYDGGFAYWGQGTLVTVSA

SEQ ID NO: 20 - protein, 231 aa, Artificial Sequence
  Description of Artificial Sequence Synthetic polypeptide
MSTESMIRDVELAEEALPKKTGGPQGSRRCLFLSLFSFLIVAGATTLFCLLHFGVIGPQREEFPRDLSLISPLAQARSSSRTPSDKVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 21 - DNA, 696 bp, Artificial Sequence
  Description of Artificial Sequence Synthetic polynucleotide
atgagcactgaaagcatgatccgggacgtggagctggccgaggaggcgctccccaagaagacaggggggccccagggctccaggcggtgcttgttcctcagcctcttctccttcctgatcgtggcaggcgccaccacgctcttctgcctgctgcactttggagtgatcggcccccagagggaagagttccccagggacctctctctaatcagccctctggcccaggcaagatcatcttctcgaaccccgagtgacaaggtagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctcgactttgccgagtctgggcaggtctactttgggatcattgccctgtcg

SEQ ID NO: 22 - protein, 220 aa, Artificial Sequence
  Description of Artificial Sequence Synthetic polypeptide
MSTESMIRDVELAEEALPKKTGGPQGSRRCLFLSLFSFLIVAGATTLFCLLHFGVIGPQREEFPRDLSLISPLAQAAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 23 - DNA, 663 bp, Artificial Sequence
  Description of Artificial Sequence Synthetic polynucleotide
atgagcactgaaagcatgatccgggacgtggagctggccgaggaggcgctccccaagaagacaggggggccccagggctccaggcggtgcttgttcctcagcctcttctccttcctgatcgtggcaggcgccaccacgctcttctgcctgctgcactttggagtgatcggcccccagagggaagagttccccagggacctctctctaatcagccctctggcccaggcagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctcgactttgccgagtctgggcaggtctactttgggatcattgccctgtcg

SEQ ID NO: 24 - protein, 231 aa, Artificial Sequence
  Description of Artificial Sequence Synthetic polypeptide
MSTESMIRDVELAEEALPKKTGGPQGSRRCLFLSLFSFLIVAGATTLFCLLHFGVIGPQREEFPRDLSLISPLAQARSSSRTPSDKPAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 25 - DNA, 696 bp, Artificial Sequence
  Description of Artificial Sequence Synthetic polynucleotide
atgagcactgaaagcatgatccgggacgtggagctggccgaggaggcgctccccaagaagacaggggggccccagggctccaggcggtgcttgttcctcagcctcttctccttcctgatcgtggcaggcgccaccacgctcttctgcctgctgcactttggagtgatcggcccccagagggaagagttccccagggacctctctctaatcagccctctggcccaggcaagatcatcttctcgaaccccgagtgacaagcctgcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctcgactttgccgagtctgggcaggtctactttgggatcattgccctgtcg

SEQ ID NO: 26 - DNA, 673 bp, Artificial Sequence
  Description of Artificial Sequence Synthetic polynucleotide
tcgagtcgagatgagcactgaaagcatgatccgggacgtggagctggccgaggaggcgctccccaagaagacaggggggccccagggctccaggcggtgcttgttcctcagcctcttctccttcctgatcgtggcaggcgccaccacgctcttctgcctgctgcactttggagtgatcggcccccagagggaagagttccccagggacctctctctaatcagccctctgcaggcagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctcgactttgccgagtctgggcaggtctactttgggatcattgccctgtcgtcg

SEQ ID NO: 27 - protein, 219 aa, Artificial Sequence
  Description of Artificial Sequence Synthetic polypeptide
MSTESMIRDVELAEEALPKKTGGPQGSRRCLFLSLFSFLIVAGATTLFCLLHFGVIGPQREEFPRDLSLISPLQAAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 28 - DNA, 654 bp, Artificial Sequence
  Description of Artificial Sequence Synthetic polynucleotide
atgagcactgaaatgcatcccggaagggggtcctggcacgaggaggcgctccccaagaagacaggggggccccagggctccaggcggtgcttgttcctcagcctcttctccttcctgatcgtggcaggcgccaccacgctcttcttcctgctgcactttggagtgatcggcccccagagggaagagttccccagggacctctctctaatcagccctctggcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcccaccagaccaaggtcaacctcctcttcgccatcaagagcccctgccagagggagaccccagagggggctgaggccaagccctggtatgagcccatctatctgggaggggtcttccagctggagaagggtgaccgactcatcgctgagatcaatcggcccgactatctctactttgccgagtatgggcaggtctactttgggatcattgccctgtcg

SEQ ID NO: 29 - protein, 218 aa, Artificial Sequence
  Description of Artificial Sequence Synthetic polypeptide
MSTEMHPGRGSWHEEALPKKTGGPQGSRRCLFLSLFSFLIVAGATTLFFLLHFGVIGPQREEFPRDLSLISPLAHVVANPQAEGQLQWLNRRANALLANGVELRDNQLVVPSEGLYLIYSQVLFKGQGCPSTHVLLTHTISRIAVSHQTKVNLLFAIKSPCQRETPEGAEAKPWYEPIYLGGVFQLEKGDRLIAEINRPDYLYFAEYGQVYFGIIALS

SEQ ID NO: 30 - protein, 233 aa, Homo sapiens
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 31 - protein, 157 aa, Homo sapiens [sequence omitted]

SEQ ID NO: 32 - protein, 33 aa, Homo sapiens
MSTESMIRDVELAEEALPKKTGGPQGSRRCLFL

SEQ ID NO: 33 - protein, 4 aa, Homo sapiens
STES

SEQ ID NO: 34 - protein, 21 aa, Homo sapiens
FSFLIVAGATTLFCLLHFGVI

SEQ ID NO: 35 - protein, 20 aa, Homo sapiens
GPQREEFPRDLSLISPLAQA

SEQ ID NO: 36 - protein, 13 aa, Homo sapiens
VRSSSRTPSDKPV

SEQ ID NO: 37 - DNA, 662 bp, Artificial Sequence
  Description of Artificial Sequence Synthetic polynucleotide
atgagcactgaaatgcatcccggaagggggtcctggcacgaggaggcgctccccaagaagacaggggggccccagggctccaggcggtgcttgttcctcagcctcttctccttcctgatcgtggcaggcgccaccacgctcttctgcctgctgcactttggagtgatcggcccccagagggaagagttccccagggacctctctctaatcagccctctggcccaggcagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctcgactttgccgagtctgggcaggtctactttgggatcattgccctgcg

SEQ ID NO: 38 - protein, 233 aa, Artificial Sequence
  Description of Artificial Sequence Synthetic polypeptide
MSTEMHPGRGSWHEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 39 - DNA, 663 bp, Artificial Sequence
  Description of Artificial Sequence Synthetic polynucleotide
atgagcactgaaagcaagatccgggacgtggagctggccgaggaggcgctccccaagaagacaggggggccccagggctccaggcggtgcttgttcctcagcctcttctccttcctgatcgtggcaggcgccaccacgctcttctgcctgctgcactttggagtgatcggcccccagagggaagagttccccagggacctctctctaatcagccctctggcccaggcagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcccaccagaccaaggtcaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctcgactttgccgagtctgggcaggtctactttgggatcattgccctgtcg

SEQ ID NO: 40 - protein, 220 aa, Artificial Sequence
  Description of Artificial Sequence Synthetic polypeptide
MSTESKIRDVELAEEALPKKTGGPQGSRRCLFLSLFSFLIVAGATTLFCLLHFGVIGPQREEFPRDLSLISPLAQAAHVVANPQAEGQLQWLNRRANALLANGVELRDNQLVVPSEGLYLIYSQVLFKGQGCPSTHVLLTHTISRIAVSHQTKVNLLSAIKSPCQRETPEGAEAKPWYEPIYLGGVFQLEKGDRLSAEINRPDYLDFAESGQVYFGIIAL

SEQ ID NO: 41 - DNA, 663 bp, Artificial Sequence
  Description of Artificial Sequence Synthetic polynucleotide
atgagcactgaaagcatgatccgggacgtggagctggccgaggaggcgctccccaagaagacaggggggccccagggctccaggcggtgcttgttcctcagcctcttctccttcctgatcgtggcaggcgccaccacgctcttcttcctgctgcactttggagtgatcggcccccagagggaagagttccccagggacctctctctaatcagccctctggcccaggcagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctcttcgccatcaagagcccctgccagagggagaccccagagggggctgaggccaagccctggtatgagcccatctatctgggaggggtcttccagctggagaagggtgaccgactcagcgctgagatcaatcggcccgactatctcgactttgccgagtctgggcaggtctactttgggatcattgccctgtcg

SEQ ID NO: 42 - protein, 220 aa, Artificial Sequence
  Description of Artificial Sequence Synthetic polypeptide
MSTESMIRDVELAEEALPKKTGGPQGSRRCLFLSLFSFLIVAGATTLFFLLHFGVIGPQREEFPRDLSLISPLAQAAHVVANPQAEGQLQWLNRRANALLANGVELRDNQLVVPSEGLYLIYSQVLFKGQGCPSTHVLLTHTISRIAVSYQTKVNLLFAIKSPCQRETPEGAEAKPWYEPIYLGGVFQLEKGDRLSAEINRPDYLDFAESGQVYFGIIAL

SEQ ID NO: 43 - DNA, 663 bp, Artificial Sequence
  Description of Artificial Sequence Synthetic polynucleotide
atgagcactgaaagcatgatccgggacgtggagctggccgaggaggcgctccccaagaagacaggggggccccagggctccaggcggtgcttgttcctcagcctcttctccttcctgatcgtggcaggcgccaccacgctcttctgcctgctgcactttggagtgatcggcccccagagggaagagttccccagggacctctctctaatcagccctctggcccaggcagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgactcatcgctgagatcaatcggcccgactatctcgactttgccgagtatgggcaggtctactttgggatcattgccctgtcg

SEQ ID NO: 44 - protein, 220 aa, Artificial Sequence
  Description of Artificial Sequence Synthetic polypeptide
MSTESMIRDVELAEEALPKKTGGPQGSRRCLFLSLFSFLIVAGATTLFCLLHFGVIGPQREEFPRDLSLISPLAQAAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLIAEINRPDYLDFAEYGQVYFGIIAL

SEQ ID NO: 45 - DNA, 663 bp, Artificial Sequence
  Description of Artificial Sequence Synthetic polynucleotide
atgagcactgaaagcatgatccgggacgtggagctggccgaggaggcgctccccaagaagacaggggggccccagggctccaggcggtgcttgttcctcagcctcttctccttcctgatcgtggcaggcgccaccacgctcttctgcctgctgcactttggagtgatcggcccccagagggaagagttccccagggacctctctctaatcagccctctggcccaggcagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctctactttgccgagtctgggcaggtctactttgggatcattgccctgtcg

SEQ ID NO: 46 - protein, 220 aa, Artificial Sequence
  Description of Artificial Sequence Synthetic polypeptide
MSTESMIRDVELAEEALPKKTGGPQGSRRCLFLSLFSFLIVAGATTLFCLLHFGVIGPQREEFPRDLSLISPLAQAAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLYFAESGQVYFGIIAL

SEQ ID NO: 47 - DNA, 660 bp, Artificial Sequence
  Description of Artificial Sequence Synthetic polynucleotide
atgagcactgaaagcatgatccgggacgtggagctggccgaggaggcgctccccaagaagacaggggggccccagggctccaggcggtgcttgttcctcagcctcttctccttcctgatcgtggcaggcgccaccacgctcttctgcctgctgcactttggagtgatcggcccccagagggaagagttccccagggacctctctctaatcagccctctgcaggcagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctctactttgccgagtctgggcaggtctactttgggatcattgccctgtcg

SEQ ID NO: 48 - protein, 219 aa, Artificial Sequence
  Description of Artificial Sequence Synthetic polypeptide
MSTESMIRDVELAEEALPKKTGGPQGSRRCLFLSLFSFLIVAGATTLFCLLHFGVIGPQREEFPRDLSLISPLAQAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLYFAESGQVYFGIIAL

SEQ ID NO: 49 - DNA, 671 bp, Artificial Sequence
  Description of Artificial Sequence Synthetic polynucleotide
atgctcgagtcgagatgagcactgaaatgcatcccggaagggggtcctggcacgaggaggcgctccccaagaagacaggggggccccagggctccaggcggtgcttgttcctcagcctcttctccttcctgatcgtggcaggcgccaccacgctcttcttcctgctgcactttggagtgatcggcccccagagggaagagttccccagggacctctctctaatcagccctctggcagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcccaccagaccaaggtcaacctcctcttcgccatcaagagcccctgccagagggagaccccagagggggctgaggccaagccctggtatgagcccatctatctgggaggggtcttccagctggagaagggtgaccgactcatcgctgagatcaatcggcccgactatctctactttgccgagtatgggcaggtctactttgggatcattgccctgtcg

SEQ ID NO: 50 - protein, 219 aa, Artificial Sequence
  Description of Artificial Sequence Synthetic polypeptide
MSTEMHPGRGSWHEEALPKKTGGPQGSRRCLFLSLFSFLIVAGATTLFFLLHFGVIGPQREEFPRDLSLISPLAQAHVVANPQAEGQLQWLNRRANALLANGVELRDNQLVVPSEGLYLIYSQVLFKGQGCPSTHVLLTHTISRIAVSHQTKVNLLFAIKSPCQRETPEGAEAKPWYEPIYLGGVFQLEKGDRLIAEINRPDYLYFAEYGQVYFGIIAL

SEQ ID NO: 51 - protein, 9 aa, Unknown
  Description of Unknown 2A peptide highly conserved sequence
  MOD_RES (5)..(5): Any amino acid
GDVEXNPGP

SEQ ID NO: 52 - protein, 21 aa, Artificial Sequence
  Description of Artificial Sequence Synthetic peptide
  MISC_FEATURE (1)..(3): May or may not be present
GSGEGRGSLLTCGDVEENPGP

SEQ ID NO: 53 - protein, 22 aa, Artificial Sequence
  Description of Artificial Sequence Synthetic peptide
  MISC_FEATURE (1)..(3): May or may not be present
GSGATNFSLLKQAGDVEENPGP

SEQ ID NO: 54 - protein, 23 aa, Artificial Sequence
  Description of Artificial Sequence Synthetic peptide
  MISC_FEATURE (1)..(3): May or may not be present
GSGQCTNYALLKLAGDVESNPGP

SEQ ID NO: 55 - protein, 25 aa, Artificial Sequence
  Description of Artificial Sequence Synthetic peptide
  MISC_FEATURE (1)..(3): May or may not be present
GSGVKQTLNFDLLKLAGDVESNPGP

SEQ ID NO: 56 - DNA, 810 bp, Artificial Sequence
  Description of Artificial Sequence Synthetic polynucleotide
atggagtttgggctgagctggctttttcttgtggctattttaaaaggtgtccagtgctctagagacgtggtgatgacccagagccacaggttcatgagcaccagcgtgggcgacagggtgagcatcacctgcagggccagccaggacgtgaacaccgccgtgagctggtaccagcagaagcccggccagagccccaagctgctgatcttcagcgccagctacaggtacaccggcgtgcccgacaggttcaccggcagcggcagcggcgccgacttcaccctgaccatcagcagcgtgcaggccgaggacctggccgtgtactactgccagcagcactacagcaccccctggaccttcggcggcggcaccaagctggacatcaagggtggtggtggttctggtggtggtggttctggcggcggcggctccggtggtggtggatcccagatccagctggtgcagagcggccccgacctgaagaagcccggcgagaccgtgaagctgagctgcaaggccagcggctacaccttcaccaacttcggcatgaactgggtgaagcaggcccccggcaagggcttcaagtggatggcctggatcaacacctacaccggcgagagctacttcgccgacgacttcaagggcaggttcgccttcagcgtggagaccagcgccaccaccgcctacctgcagatcaacaacctgaagaccgaggacaccgccacctacttctgcgccaggggcgagatctactacggctacgacggcggcttcgcctactggggccagggcaccctggtgaccgtgagcgcc

SEQ ID NO: 57 - protein, 270 aa, Artificial Sequence; identical to SEQ ID NO: 19
  Description of Artificial Sequence Synthetic polypeptide

SEQ ID NO: 58 - DNA, 1784 bp, Artificial Sequence
  Description of Artificial Sequence Synthetic polynucleotide
agactgccatgctcgagatggagtttgggctgagctggctttttcttgtggctattttaaaaggtgtccagtgctctagacagatccagctggtgcagagcggccccgacctgaagaagcccggcgagaccgtgaagctgagctgcaaggccagcggctacaccttcaccaacttcggcatgaactgggtgaagcaggcccccggcaagggcttcaagtggatggcctggatcaacacctacaccggcgagagctacttcgccgacgacttcaagggcaggttcgccttcagcgtggagaccagcgccaccaccgcctacctgcagatcaacaacctgaagaccgaggacaccgccacctacttctgcgccaggggcgagatctactacggctacgacggcggcttcgcctactggggccagggcaccctggtgaccgtgagcgccggtggtggtggttctggtggtggtggttctggcggcggcggctccggtggtggtggatccgacgtggtgatgacccagagccacaggttcatgagcaccagcgtgggcgacagggtgagcatcacctgcagggccagccaggacgtgaacaccgccgtgagctggtaccagcagaagcccggccagagccccaagctgctgatcttcagcgccagctacaggtacaccggcgtgcccgacaggttcaccggcagcggcagcggcgccgacttcaccctgaccatcagcagcgtgcaggccgaggacctggccgtgtactactgccagcagcactacagcaccccctggaccttcggcggcggcaccaagctggacatcaagcgtacggtcactgtctcttcacaggatcccgccgagcccaaatctcctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcaaccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaaaagatcccaaattttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcacgcgtgaagttc

SEQ ID NO: 59 - protein, 594 aa, Artificial Sequence
  Description of Artificial Sequence Synthetic polypeptide
TAMLEMEFGLSWLFLVAILKGVQCSRQIQLVQSGPDLKKPGETVKLSCKASGYTFTNFGMNWVKQAPGKGFKWMAWINTYTGESYFADDFKGRFAFSVETSATTAYLQINNLKTEDTATYFCARGEIYYGYDGGFAYWGQGTLVTVSAGGGGSGGGGSGGGGSGGGGSDVVMTQSHRFMSTSVGDRVSITCRASQDVNTAVSWYQQKPGQSPKLLIFSASYRYTGVPDRFTGSGSGADFTLTISSVQAEDLAVYYCQQHYSTPWTFGGGTKLDIKRTVTVSSQDPAEPKSPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKFWVLVVVGGVLACYSLLVTVAFIIFWVRSKRSRLLHSDYMNMTPRRPGPTRKHYQPYAPPRDFAAYRSRVKF

SEQ ID NO: 60 - DNA, 1766 bp, Artificial Sequence
  Description of Artificial Sequence Synthetic polynucleotide
ctcgagatggagtttgggctgagctggctttttcttgtggctattttaaaaggtgtccagtgctctagacagatacagctcgtccaatccggtcccgatttgaaaaagcctggcgaaacagttaaactgtcatgtaaggcgagcggatacacgtttacgaacttcgggatgaattgggtaaaacaggccccgggaaaaggttttaagtggatggcttggataaacacctacactggtgagtcctacttcgcagacgatttcaaagggcggttcgcgttttcagtagagacttccgccacaactgcttatctccaaataaacaacttgaagaccgaggatacggcaacctacttttgcgctcggggcgagatttattatggatatgacggcgggttcgcttactggggtcaggggacgttggttaccgtgtctgccggtggtggtggttctggtggtggtggttctggcggcggcggctccggtggtggtggatccgacgtggtgatgacgcagagccaccgattcatgagtacctctgtaggcgaccgcgtctcaattacttgtcgagcgtctcaggacgtaaatacagcggtgagctggtatcagcaaaagcccggacagagcccgaaattgctgatcttttcagcctcatacagatataccggagtcccagaccgctttacaggttccggtagtggcgcggactttactctcacaatcagctctgtacaagctgaagatttggctgtttactattgtcagcagcactatagtacgccctggaccttcgggggcggtacgaagttggatattaagcgtacggtcactgtctcttcacaggatcccgccgagcccaaatctcctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcaaccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaaaagatcccaaattttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcacgcgt

SEQ ID NO: 61 - protein, 588 aa, Artificial Sequence
  Description of Artificial Sequence Synthetic polypeptide
LEMEFGLSWLFLVAILKGVQCSRQIQLVQSGPDLKKPGETVKLSCKASGYTFTNFGMNWVKQAPGKGFKWMAWINTYTGESYFADDFKGRFAFSVETSATTAYLQINNLKTEDTATYFCARGEIYYGYDGGFAYWGQGTLVTVSAGGGGSGGGGSGGGGSGGGGSDVVMTQSHRFMSTSVGDRVSITCRASQDVNTAVSWYQQKPGQSPKLLIFSASYRYTGVPDRFTGSGSGADFTLTISSVQAEDLAVYYCQQHYSTPWTFGGGTKLDIKRTVTVSSQDPAEPKSPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKFWVLVVVGGVLACYSLLVTVAFIIFWVRSKRSRLLHSDYMNMTPRRPGPTRKHYQPYAPPRDFAAYRSR

SEQ ID NO: 62 - DNA, 827 bp, Artificial Sequence
  Description of Artificial Sequence Synthetic polynucleotide
ccatggggatgcttctcctggtgacaagccttctgctctgtgagttaccacacccagcattcctcctgatcccaggggacatcgtgctgacccagagcccccccagcctggccatgtctctgggcaagagagccaccatcagctgccgggccagcgagagcgtgaccatcctgggcagccacctgatccactggtatcagcagaagcccggccagccccccaccctgctgatccagctcgccagcaatgtgcagaccggcgtgcccgccagattcagcggcagcggcagcagaaccgacttcaccctgaccatcgaccccgtggaagaggacgacgtggccgtgtactactgcctgcagagccggaccatcccccggacctttggcggaggcaccaaactggaaatcaagggtggtggtggttctggtggtggtggttctggcggcggcggctccggtggtggtggatcccagattcagctggtgcagagcggccctgagctgaagaaacccggcgagacagtgaagatcagctgcaaggcctccggctacaccttcaccgactacagcatcaactgggtgaaaagagcccctggcaagggcctgaagtggatgggctggatcaacaccgagacaagagagcccgcctacgcctacgacttccggggcagattcgccttcagcctggaaaccagcgccagcaccgcctacctgcagatcaacaacctgaagtacgaggacaccgccacctacttttgcgccctggactacagctacgctatggactactggggccagggcaccagcgtgaccgtgtccagccgtacg

SEQ ID NO: 63 - protein, 275 aa, Artificial Sequence
  Description of Artificial Sequence Synthetic polypeptide
MGMLLLVTSLLLCELPHPAFLLIPGDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGGGGSGGGGSGGGGSGGGGSQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSRT

SEQ ID NO: 64 - DNA, 3366 bp, Artificial Sequence
  Description of Artificial Sequence Synthetic polynucleotide
atgagcactgaaatgcatcccggaagggggtcctggcacgaggaggcgctccccaagaagacaggggggccccagggctccaggcggtgcttgttcctcagcctcttctccttcctgatcgtggcaggcgccaccacgctcttcttcctgctgcactttggagtgatcggcccccagagggaagagttccccagggacctctctctaatcagccctctgcaggcagcccatgttgtagcaaaccctcaagctgaggggcagctccagtggctgaaccgccgggccaatgccctcctggccaatggcgtggagctgagagataaccagctggttgtgccatcagagggcctgtacctcatctactcccaggtcctcttcaagggccaaggctgcccctccacccatgtgctcctcacccacaccatcagccgcatcgccgtctcccaccagaccaaggtcaacctcctcttcgccatcaagagcccctgccagagggagaccccagagggggctgaggctaagccctggtatgagcccatctatctgggaggggtcttccagctggagaagggtgaccgactcatcgctgagatcaatcggcccgactatctctactttgccgagtatgggcaggtctactttgggatcattgccctgtcgtcgcgagccgagggcaggggaagtcttctaacatgcggggacgtggaggaaaatcccgggcccatggggatggccctgcctgtgacagctctgctgctgcccctggccctgctgctccatgccgccagacccgacatcgtgctgacccagagcccccccagcctggccatgtctctgggcaagagagccaccatcagctgccgggccagcgagagcgtgaccatcctgggcagccacctgatctactggtatcagcagaagcctggccagccccccaccctgctgatccagctggctagcaatgtgcagaccggcgtgcccgccagattcagcggcagcggcagcagaaccgacttcaccctgaccatcgaccccgtggaagaggacgacgtggccgtgtactactgcctgcagagccggaccatcccccggacctttggcggaggaacaaagctggaaatcaagggcagcaccagcggctccggcaagcctggctctggcgagggcagcacaaagggacagattcagctggtgcagagcggccctgagctgaagaaacccggcgagacagtgaagatcagctgcaaggcctccggctacaccttccggcactacagcatgaactgggtgaaacaggcccctggcaagggcctgaagtggatgggccggatcaacaccgagagcggcgtgcccatctacgccgacgacttcaagggcagattcgccttcagcgtggaaaccagcgccagcaccgcctacctggtgatcaacaacctgaaggacgaggataccgccagctacttctgcagcaacgactacctgtacagcctggacttctggggccagggcaccgccctgaccgtgtccagccgtacggtcactgtctcttcacaggatcccgccgagcccaaatctcctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcaaccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaaaagatcccaaattttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcacgcgtgaagttcagcaggagcgcagacgcccccgcgtaccagcagggccagaaccagctctataacgagctcaatctaggacgaagagaggagtacgatgttttggacaaaagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggaccgcagtgtactaattatgctctcttgaaattggctggagatgttgagagcaatcccgggcccatgcgcattagcaagccccacctgcggagcatcagcatccagtgctacctgtgcctgctgctgaacagccacttcctgaccgaggccggcatccacgtgttcatcctgggctgcttcagcgccggactgcccaagaccgaggccaactgggtgaacgtgatcagcgacctgaagaagatcgaggacctgatccagagcatgcacatcgacgccaccctgtacaccgagagcgacgtgcaccccagctgcaaggtgaccgccatgaagtgctttctgctggaactgcaggtgatcagcctggaaagcggcgacgccagcatccacgacaccgtggagaacctgatcatcctggccaacaacagcctgagcagcaacggcaacgtgaccgagagcggctgcaaagagtgcgaggaactggaagagaagaacatcaaagagtttctgcagagcttcgtgcacatcgtgcagatgttcatcaacaccagctga

SEQ ID NO: 65 - protein, 1121 aa, Artificial Sequence
  Description of Artificial Sequence Synthetic polypeptide
MSTEMHPGRGSWHEEALPKKTGGPQGSRRCLFLSLFSFLIVAGATTLFFLLHFGVIGPQREEFPRDLSLISPLQAAHVVANPQAEGQLQWLNRRANALLANGVELRDNQLVVPSEGLYLIYSQVLFKGQGCPSTHVLLTHTISRIAVSHQTKVNLLFAIKSPCQRETPEGAEAKPWYEPIYLGGVFQLEKGDRLIAEINRPDYLYFAEYGQVYFGIIALSSRAEGRGSLLTCGDVEENPGPMGMALPVTALLLPLALLLHAARPDIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFRHYSMNWVKQAPGKGLKWMGRINTESGVPIYADDFKGRFAFSVETSASTAYLVINNLKDEDTASYFCSNDYLYSLDFWGQGTALTVSSRTVTVSSQDPAEPKSPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGPQCTNYALLKLAGDVESNPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 66 - DNA, 1777 bp, Artificial Sequence
  Description of Artificial Sequence Synthetic polynucleotide
ccatgctcgagatggagtttgggctgagctggctttttcttgtggctattttaaaaggtgtccagtgctctagagacgtggtgatgacgcagagccaccgattcatgagtacctctgtaggcgaccgcgtctcaattacttgtcgagcgtctcaggacgtaaatacagcggtgagctggtatcagcaaaagcccggacagagcccgaaattgctgatcttttcagcctcatacagatataccggagtcccagaccgctttacaggttccggtagtggcgcggactttactctcacaatcagctctgtacaagctgaagatttggctgtttactattgtcagcagcactatagtacgccctggaccttcgggggcggtacgaagttggatattaagggtggtggtggttctggtggtggtggttctggcggcggcggctccggtggtggtggatcccagatacagctcgtccaatccggtcccgatttgaaaaagcctggcgaaacagttaaactgtcatgtaaggcgagcggatacacgtttacgaacttcgggatgaattgggtaaaacaggccccgggaaaaggttttaagtggatggcttggataaacacctacactggtgagtcctacttcgcagacgatttcaaagggcggttcgcgttttcagtagagacttccgccacaactgcttatctccaaataaacaacttgaagaccgaggatacggcaacctacttttgcgctcggggcgagatttattatggatatgacggcgggttcgcttactggggtcaggggacgttggttaccgtgtctgcccgtacggtcactgtctcttcacaggatcccgccgagcccaaatctcctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcaaccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaaaagatcccaaattttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcacgcgtgaagtt

SEQ ID NO: 67 - protein, 591 aa, Artificial Sequence
  Description of Artificial Sequence Synthetic polypeptide
MLEMEFGLSWLFLVAILKGVQCSRDVVMTQSHRFMSTSVGDRVSITCRASQDVNTAVSWYQQKPGQSPKLLIFSASYRYTGVPDRFTGSGSGADFTLTISSVQAEDLAVYYCQQHYSTPWTFGGGTKLDIKGGGGSGGGGSGGGGSGGGGSQIQLVQSGPDLKKPGETVKLSCKASGYTFTNFGMNWVKQAPGKGFKWMAWINTYTGESYFADDFKGRFAFSVETSATTAYLQINNLKTEDTATYFCARGEIYYGYDGGFAYWGQGTLVTVSARTVTVSSQDPAEPKSPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKFWVLVVVGGVLACYSLLVTVAFIIFWVRSKRSRLLHSDYMNMTPRRPGPTRKHYQPYAPPRDFAAYRSRVK

SEQ ID NO: 68 - protein, 20 aa, Artificial Sequence
  Description of Artificial Sequence Synthetic peptide
GGGGSGGGGSGGGGSGGGGS

SEQ ID NO: 69 - DNA, 60 bp, Artificial Sequence
  Description of Artificial Sequence Synthetic oligonucleotide
ggtggtggtggttctggtggtggtggttctggcggcggcggctccggtggtggtggatcc

SEQ ID NO: 70 - protein, 247 aa, Artificial Sequence
  Description of Artificial Sequence Synthetic polypeptide
RTVTVSSQDPAEPKSPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPK

SEQ ID NO: 71 - DNA, 741 bp, Artificial Sequence
  Description of Artificial Sequence Synthetic polynucleotide
cgtacggtcactgtctcttcacaggatcccgccgagcccaaatctcctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcaaccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaaaagatcccaaa

SEQ ID NO: 72 - protein, 71 aa, Artificial Sequence
  Description of Artificial Sequence Synthetic polypeptide
KFWVLVVVGGVLACYSLLVTVAFIIFWVRSKRSRLLHSDYMNMTPRRPGPTRKHYQPYAPPRDFAAYRSRV

SEQ ID NO: 73 - DNA, 210 bp, Artificial Sequence
  Description of Artificial Sequence Synthetic polynucleotide
aaattttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcacgc

SEQ ID NO: 74 - protein, 21 aa, Artificial Sequence
  Description of Artificial Sequence Synthetic peptide
MALPVTALLLPLALLLHAARP

SEQ ID NO: 75 - DNA, 63 bp, Artificial Sequence
  Description of Artificial Sequence Synthetic oligonucleotide
atggccctgcctgtgacagctctgctcctccctctggccctgctgctccatgccgccagaccc

SEQ ID NO: 76 - protein, 22 aa, Artificial Sequence
  Description of Artificial Sequence Synthetic peptide
MLLLVTSLLLCELPHPAFLLIP

SEQ ID NO: 77 - DNA, 66 bp, Artificial Sequence
  Description of Artificial Sequence Synthetic oligonucleotide
atgcttctcctggtgacaagccttctgctctgtgagttaccacacccagcattcctcctgatccca

SEQ ID NO: 78 - protein, 113 aa, Artificial Sequence
  Description of Artificial Sequence Synthetic polypeptide
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRG

SEQ ID NO: 79 - DNA, 339 bp, Artificial Sequence
  Description of Artificial Sequence Synthetic polynucleotide
cgcgtgaagttcagcaggagcgcagacgcccccgcgtaccagcagggccagaaccagctctataacgagctcaatctaggacgaagagaggagtacgatgttttggacaaaagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgga

SEQ ID NO: 80 - protein, 161 aa, Artificial Sequence
  Description of Artificial Sequence Synthetic polypeptide
RISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 81 - DNA, 483 bp, Artificial Sequence
  Description of Artificial Sequence Synthetic polynucleotide
cgcattagcaagccccacctgcggagcatcagcatccagtgctacctgtgcctgctgctgaacagccacttcctgaccgaggccggcatccacgtgttcatcctgggctgcttcagcgccggactgcccaagaccgaggccaactgggtgaacgtgatcagcgacctgaagaagatcgaggacctgatccagagcatgcacatcgacgccaccctgtacaccgagagcgacgtgcaccccagctgcaaggtgaccgccatgaagtgctttctgctggaactgcaggtgatcagcctggaaagcggcgacgccagcatccacgacaccgtggagaacctgatcatcctggccaacaacagcctgagcagcaacggcaacgtgaccgagagcggctgcaaagagtgcgaggaactggaagagaagaacatcaaagagtttctgcagagcttcgtgcacatcgtgcagatgttcatcaacaccagc

SEQ ID NO: 82 - protein, 107 aa, Artificial Sequence
  Description of Artificial Sequence Synthetic polypeptide
DVVMTQSHRFMSTSVGDRVSITCRASQDVNTAVSWYQQKPGQSPKLLIFSASYRYTGVPDRFTGSGSGADFTLTISSVQAEDLAVYYCQQHYSTPWTFGGGTKLDIK

SEQ ID NO: 83 - DNA, 321 bp, Artificial Sequence
  Description of Artificial Sequence Synthetic polynucleotide
gacgtggtgatgacccagagccacaggttcatgagcaccagcgtgggcgacagggtgagcatcacctgcagggccagccaggacgtgaacaccgccgtgagctggtaccagcagaagcccggccagagccccaagctgctgatcttcagcgccagctacaggtacaccggcgtgcccgacaggttcaccggcagcggcagcggcgccgacttcaccctgaccatcagcagcgtgcaggccgaggacctggccgtgtactactgccagcagcactacagcaccccctggaccttcggcggcggcaccaagctggacatcaag

SEQ ID NO: 84 - protein, 11 aa, Artificial Sequence
  Description of Artificial Sequence Synthetic peptide
RASQDVNTAVS

SEQ ID NO: 85 - DNA, 33 bp, Artificial Sequence
  Description of Artificial Sequence Synthetic oligonucleotide
agggccagccaggacgtgaacaccgccgtgagc

SEQ ID NO: 86 - protein, 7 aa, Artificial Sequence
  Description of Artificial Sequence Synthetic peptide
SASYRYT

SEQ ID NO: 87 - DNA, 21 bp, Artificial Sequence
  Description of Artificial Sequence Synthetic oligonucleotide
agcgccagctacaggtacacc

SEQ ID NO: 88 - protein, 9 aa, Artificial Sequence
  Description of Artificial Sequence Synthetic peptide
QQHYSTPWT

SEQ ID NO: 89 - DNA, 27 bp, Artificial Sequence
  Description of Artificial Sequence Synthetic oligonucleotide
cagcagcactacagcaccccctggacc

SEQ ID NO: 90 - protein, 122 aa, Artificial Sequence
  Description of Artificial Sequence Synthetic polypeptide
QIQLVQSGPDLKKPGETVKLSCKASGYTFTNFGMNWVKQAPGKGFKWMAWINTYTGESYFADDFKGRFAFSVETSATTAYLQINNLKTEDTATYFCARGEIYYGYDGGFAYWGQGTLVTVSA

SEQ ID NO: 91 - DNA, 366 bp, Artificial Sequence
  Description of Artificial Sequence Synthetic polynucleotide
cagatccagctggtgcagagcggccccgacctgaagaagcccggcgagaccgtgaagctgagctgcaaggccagcggctacaccttcaccaacttcggcatgaactgggtgaagcaggcccccggcaagggcttcaagtggatggcctggatcaacacctacaccggcgagagctacttcgccgacgacttcaagggcaggttcgccttcagcgtggagaccagcgccaccaccgcctacctgcagatcaacaacctgaagaccgaggacaccgccacctacttctgcgccaggggcgagatctactacggctacgacggcggcttcgcctactggggccagggcaccctggtgaccgtgagcgcc

SEQ ID NO: 92 - protein, 5 aa, Artificial Sequence
  Description of Artificial Sequence Synthetic peptide
NFGMN

SEQ ID NO: 93 - DNA, 15 bp, Artificial Sequence
  Description of Artificial Sequence Synthetic oligonucleotide
aacttcggcatgaac

SEQ ID NO: 94 - protein, 16 aa, Artificial Sequence
  Description of Artificial Sequence Synthetic peptide
INTYTGESYFADDFKG

SEQ ID NO: 95 - DNA, 48 bp, Artificial Sequence
  Description of Artificial Sequence Synthetic oligonucleotide
atcaacacctacaccggcgagagctacttcgccgacgacttcaagggc

SEQ ID NO: 96 - protein, 13 aa, Artificial Sequence
  Description of Artificial Sequence Synthetic peptide
GEIYYGYDGGFAY

SEQ ID NO: 97 - DNA, 39 bp, Artificial Sequence
  Description of Artificial Sequence Synthetic oligonucleotide
ggcgagatctactacggctacgacggcggcttcgcctac

SEQ ID NO: 98 - DNA, 366 bp, Artificial Sequence
  Description of Artificial Sequence Synthetic polynucleotide
cagatacagctcgtccaatccggtcccgatttgaaaaagcctggcgaaacagttaaactgtcatgtaaggcgagcggatacacgtttacgaacttcgggatgaattgggtaaaacaggccccgggaaaaggttttaagtggatggcttggataaacacctacactggtgagtcctacttcgcagacgatttcaaagggcggttcgcgttttcagtagagacttccgccacaactgcttatctccaaataaacaacttgaagaccgaggatacggcaacctacttttgcgctcggggcgagatttattatggatatgacggcgggttcgcttactggggtcaggggacgttggttaccgtgtctgcc

SEQ ID NO: 99 - DNA, 15 bp, Artificial Sequence
  Description of Artificial Sequence Synthetic oligonucleotide
aacttcgggatgaat

SEQ ID NO: 100 - DNA, 48 bp, Artificial Sequence
  Description of Artificial Sequence Synthetic oligonucleotide
ataaacacctacactggtgagtcctacttcgcagacgatttcaaaggg

SEQ ID NO: 101 - DNA, 39 bp, Artificial Sequence
  Description of Artificial Sequence Synthetic oligonucleotide
ggcgagatttattatggatatgacggcgggttcgcttac

SEQ ID NO: 102 - DNA, 320 bp, Artificial Sequence
  Description of Artificial Sequence Synthetic polynucleotide
acgtggtgatgacgcagagccaccgattcatgagtacctctgtaggcgaccgcgtctcaattacttgtcgagcgtctcaggacgtaaatacagcggtgagctggtatcagcaaaagcccggacagagcccgaaattgctgatcttttcagcctcatacagatataccggagtcccagaccgctttacaggttccggtagtggcgcggactttactctcacaatcagctctgtacaagctgaagatttggctgtttactattgtcagcagcactatagtacgccctggaccttcgggggcggtacgaagttggatattaag

SEQ ID NO: 103 - DNA, 33 bp, Artificial Sequence
  Description of Artificial Sequence Synthetic oligonucleotide
cgagcgtctcaggacgtaaatacagcggtgagc

SEQ ID NO: 104 - DNA, 21 bp, Artificial Sequence
  Description of Artificial Sequence Synthetic oligonucleotide
tcagcctcatacagatatacc

SEQ ID NO: 105 - DNA, 27 bp, Artificial Sequence
  Description of Artificial Sequence Synthetic oligonucleotide
cagcagcactatagtacgccctggacc

SEQ ID NO: 106 - protein, 111 aa, Artificial Sequence
  Description of Artificial Sequence Synthetic polypeptide
DIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIK

SEQ ID NO: 107 - DNA, 333 bp, Artificial Sequence
  Description of Artificial Sequence Synthetic polynucleotide
gacatcgtgctgacccagagcccccccagcctggccatgtctctgggcaagagagccaccatcagctgccgggccagcgagagcgtgaccatcctgggcagccacctgatccactggtatcagcagaagcccggccagccccccaccctgctgatccagctcgccagcaatgtgcagaccggcgtgcccgccagattcagcggcagcggcagcagaaccgacttcaccctgaccatcgaccccgtggaagaggacgacgtggccgtgtactactgcctgcagagccggaccatcccccggacctttggcggaggcaccaaactggaaatcaag

SEQ ID NO: 108 - protein, 15 aa, Artificial Sequence
  Description of Artificial Sequence Synthetic peptide
RASESVTILGSHLIH

SEQ ID NO: 109 - DNA, 45 bp, Artificial Sequence
  Description of Artificial Sequence Synthetic oligonucleotide
cgggccagcgagagcgtgaccatcctgggcagccacctgatccac

SEQ ID NO: 110 - protein, 7 aa, Artificial Sequence
  Description of Artificial Sequence Synthetic peptide
LASNVQT

SEQ ID NO: 111 - DNA, 21 bp, Artificial Sequence
  Description of Artificial Sequence Synthetic oligonucleotide
ctcgccagcaatgtgcagacc

SEQ ID NO: 112 - protein, 9 aa, Artificial Sequence
  Description of Artificial Sequence Synthetic peptide
LQSRTIPRT

SEQ ID NO: 113 - DNA, 27 bp, Artificial Sequence
  Description of Artificial Sequence Synthetic oligonucleotide
ctgcagagccggaccatcccccggacc

SEQ ID NO: 114 - protein, 117 aa, Artificial Sequence
  Description of Artificial Sequence Synthetic polypeptide
QIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 115 - DNA, 351 bp, Artificial Sequence
  Description of Artificial Sequence Synthetic polynucleotide
cagattcagctggtgcagagcggccctgagctgaagaaacccggcgagacagtgaagatcagctgcaaggcctccggctacaccttcaccgactacagcatcaactgggtgaaaagagcccctggcaagggcctgaagtggatgggctggatcaacaccgagacaagagagcccgcctacgcctacgacttccggggcagattcgccttcagcctggaaaccagcgccagcaccgcctacctgcagatcaacaacctgaagtacgaggacaccgccacctacttttgcgccctggactacagctacgctatggactactggggccagggcaccagcgtgaccgtgtccagc

SEQ ID NO: 116 - protein, 5 aa, Artificial Sequence
  Description of Artificial Sequence Synthetic peptide
DYSIN

SEQ ID NO: 117 - DNA, 15 bp, Artificial Sequence
  Description of Artificial Sequence Synthetic oligonucleotide
ctgatgtcgtagttg

SEQ ID NO: 118 - protein, 17 aa, Artificial Sequence
  Description of Artificial Sequence Synthetic peptide
WINTETREPAYAYDFRG

SEQ ID NO: 119 - DNA, 51 bp, Artificial Sequence
  Description of Artificial Sequence Synthetic oligonucleotide
tggatcaacaccgagacaagagagcccgcctacgcctacgacttccggggc

SEQ ID NO: 120 - protein, 8 aa, Artificial Sequence
  Description of Artificial Sequence Synthetic peptide
DYSYAMDY

SEQ ID NO: 121 - DNA, 24 bp, Artificial Sequence
  Description of Artificial Sequence Synthetic oligonucleotide
gactacagctacgctatggactac

SEQ ID NO: 122 - protein, 110 aa, Artificial Sequence
  Description of Artificial Sequence Synthetic polypeptide
DIVLTQSPPSLAMSLGKRATISCRASESVTILGSHLIHWYQQKPGQPPTLLIQLASNVQTVPARFSGSGSRTDFTLTIDPVEEDDVAVYYCLQSRTIPRTFGGGTKLEIK

SEQ ID NO: 123 - DNA, 333 bp, Artificial Sequence
  Description of Artificial Sequence Synthetic polynucleotide
gacattgttttgacccaatcacctccctctctcgccatgtccttgggtaaacgggcaacaatctcctgtagagcttccgaaagtgtaacaattcttggaagccacctcatacattggtatcagcaaaagccggggcagccccctacattgctcattcagttggcttcaaatgtccagacgggtgtaccagcgagattctcagggagtggctcccgaacggatttcacactgacgattgatcccgtcgaagaggacgatgtcgcagtttattattgcctccaaagtcggacaattccgaggacttttggaggcggaacaaaattggaaatcaaa

SEQ ID NO: 124 - DNA, 45 bp, Artificial Sequence
  Description of Artificial Sequence Synthetic oligonucleotide
agagcttccgaaagtgtaacaattcttggaagccacctcatacat

SEQ ID NO: 125 - DNA, 22 bp, Artificial Sequence
  Description of Artificial Sequence Synthetic oligonucleotide
ttggcttcaaatgtccagacgg

SEQ ID NO: 126 - DNA, 27 bp, Artificial Sequence
  Description of Artificial Sequence Synthetic oligonucleotide
ctccaaagtcggacaattccgaggact

SEQ ID NO: 127 - protein, 117 aa, Artificial Sequence
  Description of Artificial Sequence Synthetic polypeptide
QIQLVQSGPELKKPGETVKISCKASGYTFTDYSINWVKRAPGKGLKWMGWINTETREPAYAFDFRGRFAFSLETSASTAYLQINNLKYEDTATYFCALDYSYAMDYWGQGTSVTVSS

SEQ ID NO: 128 - DNA, 351 bp, Artificial Sequence
  Description of Artificial Sequence Synthetic polynucleotide
caaatccagctcgtccaatccggtccagagttgaagaaacccggcgagacggtaaaaatcagctgtaaagcctcaggttacacgtttacggactatagcattaattgggttaagagggctccggggaaggggctcaaatggatgggctggataaacacagagacgagagagcccgcatatgcgttcgactttagaggtcgattcgctttcagtcttgaaacctctgcttctaccgcgtatctccagataaacaacctgaaatatgaggatacagcaacttatttttgcgctctcgattacagctatgcgatggattattggggacaaggaacttccgtgactgtgtcaagc

SEQ ID NO: 129 - DNA, 15 bp, Artificial Sequence
  Description of Artificial Sequence Synthetic oligonucleotide
gactatagcattaat

SEQ ID NO: 130 - protein, 17 aa, Artificial Sequence
  Description of Artificial Sequence Synthetic peptide
WINTETREPAYAFDFRG

SEQ ID NO: 131 - DNA, 51 bp, Artificial Sequence
  Description of Artificial Sequence Synthetic oligonucleotide
tggataaacacagagacgagagagcccgcatatgcgttcgactttagaggt

SEQ ID NO: 132 - DNA, 24 bp, Artificial Sequence
  Description of Artificial Sequence Synthetic oligonucleotide
gattacagctatgcgatggattat

SEQ ID NO: 133 - protein, 111 aa, Artificial Sequence
  Description of Artificial Sequence Synthetic polypeptide
DIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIK

SEQ ID NO: 134 - DNA, 333 bp, Artificial Sequence
  Description of Artificial Sequence Synthetic polynucleotide
gacatcgtgctgacccagagcccccccagcctggccatgtctctgggcaagagagccaccatcagctgccgggccagcgagagcgtgaccatcctgggcagccacctgatctactggtatcagcagaagcctggccagccccccaccctgctgatccagctggctagcaatgtgcagaccggcgtgcccgccagattcagcggcagcggcagcagaaccgacttcaccctgaccatcgaccccgtggaagaggacgacgtggccgtgtactactgcctgcagagccggaccatcccccggacctttggcggaggaacaaagctggaaatcaag

SEQ ID NO: 135 - protein, 14 aa, Artificial Sequence
  Description of Artificial Sequence Synthetic peptide
ASESVTILGSHLIY

SEQ ID NO: 136 - DNA, 41 bp, Artificial Sequence
  Description of Artificial Sequence Synthetic oligonucleotide
ccagcgagagcgtgaccatcctgggcagccacctgatctac

SEQ ID NO: 137 - protein, 6 aa, Artificial Sequence
  Description of Artificial Sequence Synthetic peptide
ASNVQT

SEQ ID NO: 138 - DNA, 18 bp, Artificial Sequence
  Description of Artificial Sequence Synthetic oligonucleotide
gctagcaatgtgcagacc

SEQ ID NO: 139 - protein, 9 aa, Artificial Sequence
  Description of Artificial Sequence Synthetic peptide
LQSRTIPRT

SEQ ID NO: 140 - DNA, 27 bp, Artificial Sequence
  Description of Artificial Sequence Synthetic oligonucleotide
ctgcagagccggaccatcccccggacc

SEQ ID NO: 141 - protein, 117 aa, Artificial Sequence
  Description of Artificial Sequence Synthetic polypeptide
QIQLVQSGPELKKPGETVKISCKASGYTFRHYSMNWVKQAPGKGLKWMGRINTESGVPIYADDFKGRFAFSVETSASTAYLVINNLKDEDTASYFCSNDYLYSLDFWGQGTALTVSS

SEQ ID NO: 142 - DNA, 351 bp, Artificial Sequence
  Description of Artificial Sequence Synthetic polynucleotide
cagattcagctggtgcagagcggccctgagctgaagaaacccggcgagacagtgaagatcagctgcaaggcctccggctacaccttccggcactacagcatgaactgggtgaaacaggcccctggcaagggcctgaagtggatgggccggatcaacaccgagagcggcgtgcccatctacgccgacgacttcaagggcagattcgccttcagcgtggaaaccagcgccagcaccgcctacctggtgatcaacaacctgaaggacgaggataccgccagctacttctgcagcaacgactacctgtacagcctggacttctggggccagggcaccgccctgaccgtgtccagc

SEQ ID NO: 143 - protein, 5 aa, Artificial Sequence
  Description of Artificial Sequence Synthetic peptide
HYSMN

SEQ ID NO: 144 - DNA, 15 bp, Artificial Sequence
  Description of Artificial Sequence Synthetic oligonucleotide
cactacagcatgaac

SEQ ID NO: 145 - protein, 17 aa, Artificial Sequence
  Description of Artificial Sequence Synthetic peptide
RINTESGVPIYADDFKG

SEQ ID NO: 146 - DNA, 51 bp, Artificial Sequence
  Description of Artificial Sequence Synthetic oligonucleotide
cggatcaacaccgagagcggcgtgcccatctacgccgacgacttcaagggc

SEQ ID NO: 147 - protein, 7 aa, Artificial Sequence
  Description of Artificial Sequence Synthetic peptide
YLYSLDF

SEQ ID NO: 148 - DNA, 21 bp, Artificial Sequence
  Description of Artificial Sequence Synthetic oligonucleotide
tacctgtacagcctggacttc

SEQ ID NO: 149 - protein, 220 aa, Artificial Sequence
  Description of Artificial Sequence Synthetic polypeptide
MSTEMHPGRGSWHEEALPKKTGGPQGSRRCLFLSLFSFLIVAGATTLFFLLHFGVIGPQREEFPRDLSLISPLQAAHVVANPQAEGQLQWLNRRANALLANGVELRDNQLVVPSEGLYLIYSQVLFKGQGCPSTHVLLTHTISRIAVSHQTKVNLLFAIKSPCQRETPEGAEAKPWYEPIYLGGVFQLEKGDRLIAEINRPDYLYFAEYGQVYFGIIALS

SEQ ID NO: 150 - DNA, 661 bp, Artificial Sequence
  Description of Artificial Sequence Synthetic polynucleotide
atgagcactgaaatgcatcccggaagggggtcctggcacgaggaggcgctccccaagaagacaggggggccccagggctccaggcggtgcttgttcctcagcctcttctccttcctgatcgtggcaggcgccaccacgctcttcttcctgctgcactttggagtgatcggcccccagagggaagagttccccagggacctctctctaatcagccctctgcaggcagcccatgttgtagcaaaccctcaagctgaggggcagctccagtggctgaaccgccgggccaatgccctcctggccaatggcgtggagctgagagataaccagctggttgtgccatcagagggcctgtacctcatctactcccaggtcctcttcaagggccaaggctgcccctccacccatgtgctcctcacccacaccatcagccgcatcgccgtctcccaccagaccaaggtcaacctcctcttcgccatcaagagcccctgccagagggagaccccagagggggctgaggctaagccctggtatgagcccatctatctgggaggggtcttccagctggagaagggtgaccgactcatcgctgagatcaatcggcccgactatctctactttgccgagtatgggcaggtctactttgggatcattgccctgtcgt

SEQ ID NO: 151 - DNA, 1766 bp, Artificial Sequence; identical to SEQ ID NO: 60
  Description of Artificial Sequence Synthetic polynucleotide

SEQ ID NO: 152 - protein, 588 aa, Artificial Sequence; identical to SEQ ID NO: 61
  Description of Artificial Sequence Synthetic polypeptide

SEQ ID NO: 153 - DNA, 3366 bp, Artificial Sequence
  Description of Artificial Sequence Synthetic polynucleotide
atgagcactgaaatgcatcccggaagggggtcctggcacgaggaggcgctccccaagaagacaggggggccccagggctccaggcggtgcttgttcctcagcctcttctccttcctgatcgtggcaggcgccaccacgctcttcttcctgctgcactttggagtgatcggcccccagagggaagagttccccagggacctctctctaatcagccctctgcaggcagcccatgttgtagcaaaccctcaagctgaggggcagctccagtggctgaaccgccgggccaatgccctcctggccaatggcgtggagctgagagataaccagctggttgtgccatcagagggcctgtacctcatctactcccaggtcctcttcaagggccaaggctgcccctccacccatgtgctcctcacccacaccatcagccgcatcgccgtctcccaccagaccaaggtcaacctcctcttcgccatcaagagcccctgccagagggagaccccagagggggctgaggctaagccctggtatgagcccatctatctgggaggggtcttccagctggagaagggtgaccgactcatcgctgagatcaatcggcccgactatctctactttgccgagtatgggcaggtctactttgggatcattgccctgtcgtcgcgagccgagggcaggggaagtcttctaacatgcggggacgtggaggaaaatcccgggcccatggggatggccctgcctgtgacagctctgctcctccctctggccctgctgctccatgccgccagacccgacatcgtgctgacccagagcccccccagcctggccatgtctctgggcaagagagccaccatcagctgccgggccagcgagagcgtgaccatcctgggcagccacctgatccactggtatcagcagaagcccggccagccccccaccctgctgatccagctcgccagcaatgtgcagaccggcgtgcccgccagattcagcggcagcggcagcagaaccgacttcaccctgaccatcgaccccgtggaagaggacgacgtggccgtgtactactgcctgcagagccggaccatcccccggacctttggcggaggcaccaaactggaaatcaagggcagcaccagcggctccggcaagcctggctctggcgagggcagcacaaagggacagattcagctggtgcagagcggccctgagctgaagaaacccggcgagacagtgaagatcagctgcaaggcctccggctacaccttcaccgactacagcatcaactgggtgaaaagagcccctggcaagggcctgaagtggatgggctggatcaacaccgagacaagagagcccgcctacgcctacgacttccggggcagattcgccttcagcctggaaaccagcgccagcaccgcctacctgcagatcaacaacctgaagtacgaggacaccgccacctacttttgcgccctggactacagctacgctatggactactggggccagggcaccagcgtgaccgtgtccagccgtacggtcactgtctcttcacaggatcccgccgagcccaaatctcctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcaaccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaaaagatcccaaattttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcacgcgtgaagttcagcaggagcgcagacgcccccgcgtaccagcagggccagaaccagctctataacgagctcaatctaggacgaagagaggagtacgatgttttggacaaaagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggaccgcagtgtactaattatgctctcttgaaattggctggagatgttgagagcaatcccgggcccatgcgcattagcaagccccacctgcggagcatcagcatccagtgctacctgtgcctgctgctgaacagccacttcctgaccgaggccggcatccacgtgttcatcctgggctgcttcagcgccggactgcccaagaccgaggccaactgggtgaacgtgatcagcgacctgaagaagatcgaggacctgatccagagcatgcacatcgacgccaccctgtacaccgagagcgacgtgcaccccagctgcaaggtgaccgccatgaagtgctttctgctggaactgcaggtgatcagcctggaaagcggcgacgccagcatccacgacaccgtggagaacctgatcatcctggccaacaacagcctgagcagcaacggcaacgtgaccgagagcggctgcaaagagtgcgaggaactggaagagaagaacatcaaagagtttctgcagagcttcgtgcacatcgtgcagatgttcatcaacaccagctga

SEQ ID NO: 154 - protein, 1121 aa, Artificial Sequence
  Description of Artificial Sequence Synthetic polypeptide
MSTEMHPGRGSWHEEALPKKTGGPQGSRRCLFLSLFSFLIVAGATTLFFLLHFGVIGPQREEFPRDLSLISPLQAAHVVANPQAEGQLQWLNRRANALLANGVELRDNQLVVPSEGLYLIYSQVLFKGQGCPSTHVLLTHTISRIAVSHQTKVNLLFAIKSPCQRETPEGAEAKPWYEPIYLGGVFQLEKGDRLIAEINRPDYLYFAEYGQVYFGIIALSSRAEGRGSLLTCGDVEENPGPMGMALPVTALLLPLALLLHAARPDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSRTVTVSSQDPAEPKSPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGPQCTNYALLKLAGDVESNPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 155 - DNA, 821 bp, Artificial Sequence
  Description of Artificial Sequence Synthetic polynucleotide
ccatggggatgcttctcctggtgacaagccttctgctctgtgagttaccacacccagcattcctcctgatcccagggcaaatccagctcgtccaatccggtccagagttgaagaaacccggcgagacggtaaaaatcagctgtaaagcctcaggttacacgtttacggactatagcatcaattgggttaagagggctccggggaaggggctcaaatggatgggctggataaacacagagacgagagagcccgcatatgcgtacgactttagaggtcgattcgctttcagtcttgaaacctctgcttctaccgcgtatctccagataaacaacctgaaatatgaggatacagcaacttatttttgcgctctcgattacagctatgcgatggattattggggacaaggaacttccgtgactgtgtcaagcgggggtggaggttctggcggagggggcagcggtggtggaggaagtgggggcggtgggagtgacattgttttgacccaatcacctccctctctcgccatgtccttgggtaaacgggcaacaatctcctgtagagcttccgaaagtgtaacaattcttggaagccacctcatacattggtatcagcaaaagccggggcagccccctacattgctcattcaattggcttcaaatgtccagacgggtgtaccagcgagattctcagggagtggctcccgaacggatttcacactgacgattgatcccgtcgaagaggacgatgtcgcagtttattattgcctccaaagtcggacaattccgaggacttttggaggcggaacaaaattggaaatcaaa

SEQ ID NO: 156 - protein, 273 aa, Artificial Sequence
  Description of Artificial Sequence Synthetic polypeptide
MGMLLLVTSLLLCELPHPAFLLIPGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSGGGGSGGGGSGGGGSGGGGSDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIK

SEQ ID NO: 157 - DNA, 3366 bp, Artificial Sequence; identical to SEQ ID NO: 153
  Description of Artificial Sequence Synthetic polynucleotide

SEQ ID NO: 158 - protein, 1121 aa, Artificial Sequence; identical to SEQ ID NO: 154
  Description of Artificial Sequence Synthetic polypeptide

(19) United States
(12) Patent Application Publication
(10) Pub. No.: US 2022/0370500 A1
(43) Pub. Date: Nov. 24, 2022

(54) A METHOD OF ENGINEERING NATURAL KILLER-CELLS TO TARGET BCMA-POSITIVE TUMORS

(71) Applicant: BOARD OF REGENTS, THE UNIVERSITY OF TEXAS SYSTEM, Austin, TX (US)

(72) Inventors: Katy REZVANI, Houston, TX (US); David MARIN COSTA, Houston, TX (US)

(73) Assignee: BOARD OF REGENTS, THE UNIVERSITY OF TEXAS SYSTEM, Austin, TX (US)

(21) Appl. No.: 17/761,190

(22) PCT Filed: Sep. 15, 2020
(86) PCT No.: PCT/US2020/050864

§ 371 Date: Mar. 16, 2022

Related U.S. Application Data
(60) Provisional application No. 62/902,237, filed on Sep. 18, 2019.

(51) Int. Cl. A61K 35/17; A61P 35/00

(52) U.S. Cl. CPC A61K 35/17; A61P 35/00

(57) ABSTRACT

Embodiments of the disclosure include methods and compositions related to targeting of BCMA-expressing cells by NK cells specifically engineered to bind the BCMA antigen. In particular embodiments, NK cells that are manipulated to expressing BCMA-targeting chimeric antigen receptors (CARs) are utilized to target cancers that express BCMA. In certain embodiments, vectors that express the BCMA-targeting CARs also express particular suicide genes and/or particular cytokines.

This application claims priority to U.S. Provisional Patent Application Ser. No. 62/902,237, filed Sep. 18, 2019, which is incorporated by reference herein in its entirety.

### TECHNICAL FIELD

Embodiments of the disclosure include at least the fields of cell biology, molecular biology, immunology, and medicine, including cancer medicine.

### BACKGROUND

Genetic reprogramming of Natural Killer (NK) cells for adoptive cancer immunotherapy has clinically relevant applications and benefits such as 1) innate anti-tumor surveillance without prior need for sensitization; 2) allogeneic efficacy without graft versus host reactivity; and 3) direct cell-mediated cytotoxicity and cytolysis of target tumors. Human NK cell development and acquisition of self-tolerance, alloreactivity, and effector functions is an adaptive process of licensing, calibration, and arming. At the molecular level, specific activating and inhibitory receptors direct NK cellular functions by aggregating, balancing, and integrating extracellular signals into distinct effector functions. The functional activity of NK cells and responsiveness to extrinsic stimuli follow the ‘rheostat’ model of continuous education and thus amenable to reprogramming. Genetic modification of NK cells to redirect their effector functions is an effective method to harness their cytotoxic capability to kill tumor cells.

The present disclosure provides a solution to long felt needs in the art of treating cancer effectively.

### BRIEF SUMMARY

The present disclosure is directed to methods and compositions related to cancers in which targeting of cancer cells through B Cell Maturation Antigen (BCMA) would be effective. In particular embodiments, the present disclosure is directed to methods and compositions related to treatment of BCMA-positive cancers, and in at least certain cases the BCMA-positive cancers are targeted through the use of natural killer (NK) cells.

The present disclosure provides methods and compositions for the treatment of cancer patients with BCMA-positive cancers (for example, B cell malignancies, multiple myeloma, head and neck cancer, lung cancer, thyroid cancer, or breast cancer) including through the ablation of BCMA-expressing cancer cells.

In particular embodiments, the presently disclosed methods and compositions allow for the use of off-the-shelf NK cells that in specific embodiments are also transduced to express one or more cytokines, such as IL-15, IL-12, IL-18, IL-2, and/or IL-21.

Encompassed herein are methods to genetically engineer mammalian NK cells, including human NK cells, to target BCMA-positive tumors of any kind, including at least myeloma. The disclosure encompasses a number of examples of chimeric antigen receptor (CAR) constructs that target BCMA that may be expressed on multiple myeloma cancer cells as well as other B cell malignancies and other cancers, including at least lung and breast cancer. In specific embodiments, the present disclosure provides a number of expression constructs (including retroviral constructs) that express a single chain variable fragment (scFv) against BCMA and, in some embodiments, the constructs include cytokines such as IL-15 (as one example) to support NK cell survival and proliferation. The cytokine(s) are not part of the CAR molecule, in specific embodiments. In a series of in vitro studies provided herein, the activity of anti-BCMA.CAR/IL-15 transduced cord blood (CB)-NK cells against myeloma cell lines is demonstrated.

In particular embodiments, the NK cells of the disclosure harboring one or more vectors that encode CARs that target BCMA also have a vector that encodes a suicide gene. The vector that encodes the CAR may or may not also encode the suicide gene (and may or may not encode the cytokine). In particular embodiments, the suicide gene is a mutant TNFalpha, including a mutant TNFalpha that is nonsecretable and engineered by the hand of man.

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention. Aspects of an embodiment set forth in the Examples are also embodiments that may be implemented in the context of embodiments discussed elsewhere in a different Example or elsewhere in the application, such as in the Brief Summary, Detailed Description, Claims, and Brief Description of the Drawings.

The foregoing has outlined rather broadly the features and technical advantages of the present disclosure in order that the detailed description that follows may be better understood. Additional features and advantages will be described hereinafter which form the subject of the claims herein. It should be appreciated by those skilled in the art that the conception and specific embodiments disclosed may be readily utilized as a basis for modifying or designing other structures for carrying out the same purposes of the present designs. It should also be realized by those skilled in the art that such equivalent constructions do not depart from the spirit and scope as set forth in the appended claims. The novel features which are believed to be characteristic of the designs disclosed herein, both as to the organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

### BRIEF DESCRIPTION OF THE DRAWINGS

For a more complete understanding of the present disclosure, reference is now made to the following descriptions taken in conjunction with the accompanying drawings.

FIG. 1 is an illustration of a vector that encodes a BCMA-targeting chimeric antigen receptor (CAR) utilizing codon optimized (co) C11D5.3 scFv VL and VH chains, and a granulocyte-macrophage colony-stimulating factor receptor (GMCSFR) signal peptide. A linker links the VH and VL chains.

FIG. 2 illustrates a vector that comprises TNF-alpha mutant suicide gene separated by a 2A element from a BCMA-targeting CAR at the 5′ end of the sequence that encodes the CAR, and the CAR is also separated from IL-15 with another 2A element at the 3′ end of the sequence that encodes the CAR. The BMCA-targeting CAR includes codon optimized C12A3.2 scFv VH and VL chains, the IgG1 hinge, CD28 costimulatory domain, and CD3zeta.

FIG. 3 exemplifies a vector encoding a BCMA-targeting CAR that includes the CD8alpha signal peptide, the C11D5.3 scFV VL and VH chains, IgG1 hinge and CD28 costimulatory domain and CD3zeta. The CAR is separated by IL15 with a 2A element.

FIG. 4 is an illustration of a vector that encodes a BCMA-targeting CAR that employs the codon-optimized C12A3.2 scFv VH and VL chains, the IgG1 linker, CD28 costimulatory domain, and CD3 zeta. The CAR is also separated from IL15 by a 2A element.

FIG. 5 shows a vector that encodes a codon-optimized BCMA-targeting CAR with the A7D12.2VH chain of the antibody upstream in a 5′ to 3′ direction from the A7D12.2 VL chain, in addition to the IgG1 hinge, CD28 costimulatory domain, and CD3zeta. The CAR also includes the Ig Heavy chain signal peptide and is separated from IL15 by a 2A element.

FIG. 6 shows a vector that encodes a BCMA-targeting CAR with the codon-optimized A7D12.2VL chain of the antibody upstream in a 5′ to 3′ direction from the A7D12.2 VH chain, in addition to the IgG1 hinge, CD28 costimulatory domain, and CD3zeta. The CAR also includes the Ig Heavy chain signal peptide and is separated from IL15 by a 2A element.

FIG. 7 provides one example of an expression vector that encodes a BCMA-targeting CAR with A7D12.2 VL chain linked in a 5′ to 3′ direction to A7D12.2 VH chain and also including the Ig heavy chain signal peptide.

FIG. 8 illustrates an example of an expression vector that encodes a BCMA-targeting CAR with A7D12.2 VH chain linked in a 5′ to 3′ direction to A7D12.2 VL chain and the IgG1 hinge. The CAR utilizes the Ig heavy chain signal peptide and the CD28 costimulatory domains.

FIG. 9 provides an illustration of an expression vector that encodes a BCMA-targeting CAR with codon-optimized A7D12.2 VH chain linked in a 5′ to 3′ direction to codon-optimized A7D12.2 VL chain and utilizing an Ig heavy chain signal peptide, an IgG1 hinge and CD28 costimulatory domain.

FIG. 10 is an illustration of an expression vector that encodes a BCMA-targeting CAR with C11D5.3 VL chain linked in a 5′ to 3′ direction to C11D5.3 VH chain, with the CAR utilizing GMCSF-R signal peptide.

FIG. 11 shows an illustration of a plasmid map of an expression vector encoding a BCMA-targeting CAR utilizing codon-optimized (CO) C12A3.2 VL chain linked in a 5′ to 3′ direction to codon-optimized C12A3.2 VH chain, wherein the CAR incorporates the CD8 signal peptide, the IgG1 hinge, CD28, and CD3zeta. The vector also encodes a particular TNFalpha mutant, delAla-1 to Val13 (14aa del) CKI mutant 5aa mut and encodes IL15. IL15 and the TNFalpha mutant are separated from the CAR by 2A peptides sequences.

FIG. 12 provides an illustration of an expression vector that encodes a BCMA-targeting CAR with codon optimized A7D12.2 VL linked in a 5′ to 3′ direction to A7D12.2 VH and utilizing the Ig heavy chain signal peptide, IgG1 hinge, and CD28 costimulatory domain.

FIG. 13 shows an illustration of an expression vector that encodes a BCMA-targeting CAR with an Ig Heavy Chain signal peptide, codon optimized A7D12.2 VH chain linked to codon optimized A7D12.2 VL chain in a 5′ to 3′ direction, in addition to IgG1 hinge and CD28. The CDR sequences for the VH and VL chains are illustrated.

FIG. 14 illustrates an expression vector that encodes a BCMA-targeting CAR with C11D5.3 VL chain linked in a 5′ to 3′ direction to the C11D5.3 VH chain and also including CD8a signal peptide and IgG1 hinge.

FIG. 15 provides an illustration of an expression vector that encodes a BCMA-targeting CAR with C11D5.3 VH chain linked in a 5′ to 3′ direction to the C11D5.3 VL chain, wherein the CAR employs that GMCSF-R signal peptide. The CDRs of the corresponding VH and VL chains are illustrated.

FIG. 16 shows an illustration of an expression vector that encodes a BCMA-targeting CAR with C11D5.3 VL chain is linked in a 5′ to 3′ direction to the C11D5.3 VH chain, and wherein the CAR employs IgG1 hinge, CD28, and CD3z. The CDRs of the corresponding VH and VL chains are illustrated. The construct also encodes a TNFalpha mutant and IL15, each separated from the CAR sequence by a 2A peptide sequence.

FIGS. 17A-17B indicate the cytotoxicity of NK cells transduced with 5 different BCMA constructs against the myeloma cell line MM1S. BCMA1 is IgSPCOA7D12VLVH28Z15 (a construct comprising Ig Heavy Chain Signal Peptide; codon optimized A7D12 light chain that is 5′ to codon optimized A7D12 heavy chain; CD28 costimulatory domain; CD3 zeta chain; and IL-15); BCMA2 is CD8SPC11D5.3VLVH15 (a construct comprising CD8 Signal Peptide; the 11D5.3 scFv light chain that is 5′ to 11D5.3 heavy chain; CD28 costimulatory domain; CD3 zeta chain; and IL-15); BCMA3 is COGSPC11D5.3VLVHZIL15 (a construct comprising GM-CSF Signal Peptide; codon optimized 11D5.3 light chain that is 5′ to codon optimized 11D5.3 heavy chain; CD28 costimulatory domain; CD3 zeta chain; and IL-15); BCMA4 is IgSPA7D12VHVL28Z15 (a construct comprising Ig Heavy Chain Signal Peptide; A7D12 heavy chain that is 5′ to A7D12 light chain; CD28 costimulatory domain; CD3 zeta chain; and IL-15); and BCMA5 is IgSPA7D12VLVH28Z15 (a construct comprising Ig Heavy Chain Signal Peptide; A7D12 light chain that is 5′ to A7D12 heavy chain; CD28 costimulatory domain; CD3 zeta chain; and IL-15).

FIG. 18 demonstrates cytotoxicity of T cells transduced with five different BCMA constructs against the myeloma cell line MM1.S compared to control. From left to right in the groupings of bars: BCMA1 is IgSPCOA7D12VLVH28Z15; BCMA2 is CD8SPC11D51VLVH15; BCMA3 is COGSPC11D51VLVHZIL15; BCMA4 is IgSPA7D12VHVL28Z15; and BCMA5 is IgSPA7D12VLVH28Z15.Control is “empty” virus. No target plasmid, only the two helper plasmids when making the virus.

FIG. 19 demonstrates BCMA surface expression on multiple myeloma cell lines (MM1S, H929, and RPMI 8226).

FIG. 20 shows a chromium assay for BCMA CAR NK cell cytotoxicity against multiple myeloma targets (MM1S, H929, RPMI 8226). BCMA1-5 are the same constructs utilized as in FIG. 18, and the control is non-transduced (NT) cells.

FIG. 21 demonstrates that silencing BCMA by CRISPR deletion in MM1S eliminates enhanced killing from CAR BCMA NK cells.

FIG. 22 shows production of particular effector cytokines by BCMA CAR NK cells when co-cultured with MM1S or H929 targets.

FIG. 23 illustrates an example of a BCMA mouse experimental plan to characterize the ability of BCMA CAR NK cells to control MM1S tumor in vivo.

FIG. 24 shows BCMA transduction efficiency with various constructs BCMA-1 through BCMA-5.

FIG. 25 demonstrates BCMA CAR NK cell antitumor activity in a FFluc-MM1S mouse model based on bioluminescence imaging.

FIG. 26 shows BCMA CAR NK cells antitumor activity in MM1S mouse model as a function of survival.

### DETAILED DESCRIPTION

The following applications are incorporated by reference herein in their entirety: PCT/US2019/018989; U.S. Provisional Patent Application No. 62/769,405, filed Nov. 19, 2018; U.S. Provisional Patent Application No. 62/773,372, filed Nov. 30, 2018; U.S. Provisional Patent Application No. 62/791,464, filed Jan. 11, 2019; U.S. Provisional Patent Application No. 62/769,414, filed Nov. 19, 2018; U.S. Provisional Patent Application No. 62/773,394, filed Nov. 30, 2019; and U.S. Provisional Patent Application No. 62/791,491, filed Jan. 11, 2019. While various embodiments of the disclosure have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the disclosure described herein may be employed.

### 1. Examples of Definitions

In keeping with long-standing patent law convention, the words “a” and “an” when used in the present specification in concert with the word comprising, including the claims, denote “one or more.” Some embodiments of the disclosure may consist of or consist essentially of one or more elements, method steps, and/or methods of the disclosure. It is contemplated that any method or composition described herein can be implemented with respect to any other method or composition described herein and that different embodiments may be combined.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

Throughout this application, the term “about” is used according to its plain and ordinary meaning in the area of cell and molecular biology to indicate that a value includes the standard deviation of error for the device or method being employed to determine the value.

The term “engineered” as used herein refers to an entity that is generated by the hand of man, including a cell, nucleic acid, polypeptide, vector, and so forth. In at least some cases, an engineered entity is synthetic and comprises elements that are not naturally present or configured in the manner in which it is utilized in the disclosure. In specific embodiments, a vector is engineered through recombinant nucleic acid technologies, and a cell is engineered through transfection or transduction of an engineered vector.

As used herein, “prevent,” and similar words such as “prevented,” “preventing” etc., indicate an approach for preventing, inhibiting, or reducing the likelihood of the occurrence or recurrence of, a disease or condition, e.g., cancer. It also refers to delaying the onset or recurrence of a disease or condition or delaying the occurrence or recurrence of the symptoms of a disease or condition. As used herein, “prevention” and similar words also includes reducing the intensity, effect, symptoms and/or burden of a disease or condition prior to onset or recurrence of the disease or condition.

The term “sample,” as used herein, generally refers to a biological sample. The sample may be taken from tissue or cells from an individual. In some examples, the sample may comprise, or be derived from, a tissue biopsy, blood (e.g., whole blood), blood plasma, extracellular fluid, dried blood spots, cultured cells, discarded tissue. The sample may have been isolated from the source prior to collection. Non-limiting examples include blood, serum, plasma, cerebral spinal fluid, pleural fluid, amniotic fluid, lymph fluid, saliva, urine, stool, tears, sweat, bone marrow, or mucosal excretions, and other bodily fluids isolated from the primary source prior to collection. In some examples, the sample is isolated from its primary source (cells, tissue, bodily fluids such as blood, environmental samples, etc.) during sample preparation. The sample may or may not be purified or otherwise enriched from its primary source. In some cases the primary source is homogenized prior to further processing. The sample may be filtered or centrifuged to remove buffy coat, lipids, or particulate matter. The sample may also be purified or enriched for nucleic acids, or may be treated with RNases. The sample may contain tissues or cells that are intact, fragmented, or partially degraded.

The term “subject,” as used herein, generally refers to an individual having a biological sample that is undergoing processing or analysis and, in specific cases, has or is suspected of having cancer. The subject can be any organism or animal subject that is an object of a method or material, including mammals, e.g., humans, laboratory animals (e.g., primates, rats, mice, rabbits), livestock (e.g., cows, sheep, goats, pigs, turkeys, and chickens), household pets (e.g., dogs, cats, and rodents), horses, and transgenic non-human animals. The subject can be a patient, e.g., have or be suspected of having a disease (that may be referred to as a medical condition), such as benign or malignant neoplasias, or cancer. The subject may being undergoing or having undergone treatment. The subject may be asymptomatic. The subject may be healthy individuals but that are desirous of prevention of cancer. The term “individual” may be used interchangeably, in at least some cases. The “subject” or “individual”, as used herein, may or may not be housed in a medical facility and may be treated as an outpatient of a medical facility. The individual may be receiving one or more medical compositions via the internet. An individual may comprise any age of a human or non-human animal and therefore includes both adult and juveniles (i.e., children) and infants and includes in utero individuals. It is not intended that the term connote a need for medical treatment, therefore, an individual may voluntarily or involuntarily be part of experimentation whether clinical or in support of basic science studies.

As used herein “treatment” or “treating,” includes any beneficial or desirable effect on the symptoms or pathology of a disease or pathological condition, and may include even minimal reductions in one or more measurable markers of the disease or condition being treated, e.g., cancer. Treatment can involve optionally either the reduction or amelioration of symptoms of the disease or condition, or the delaying of the progression of the disease or condition. “Treatment” does not necessarily indicate complete eradication or cure of the disease or condition, or associated symptoms thereof.

The present disclosure encompasses BCMA-targeting cells, including NK cells manipulated to express a BCMA-targeting CAR and optionally wherein the NK cells express a suicide gene (such as a nonsecretable mutant TNFalpha) and optionally one or more cytokines. In particular embodiments, NK cells express a BCMA-targeting CAR, a mutant nonsecretable TNFalpha, and at least one cytokine.

The skilled artisan recognizes that BCMA is also known as tumor necrosis factor receptor superfamily member 17 (TNFRSF17); CD269; TNFRSF13A; and TNF receptor superfamily member 17.

### I. EXAMPLES OF CAR EMBODIMENTS

In particular embodiments, the disclosure concerns the reprogramming of NK cells (for example, cord blood (CB)-derived NK cells) to target cancer cells expressing BCMA. The disclosure provides a number of novel CAR constructs incorporating different BCMA scFvs heterologously fused to a signaling domain comprising cytoplasmic portions of CD247 (also known as CD3) and CD28. In alternative embodiments, other costimulatory domain(s) besides CD28 are utilized. In particular embodiments, the scFv is a fusion of the variable fragments derived from the heavy (V_{H}) and light (V_{L}) chains of a murine antibody with specificity for human BCMA antigen. The scFv has been codon optimized, in particular embodiments. In specific embodiments, the vector also carries a cytokine gene, for example IL-15, to produce human interleukins. IL-15, as one example, aids in the survival and maintenance of NK cells. The cells, thus modified and in one embodiment, may be referred to herein as CAR.BCMA.CD28.CD3z-IL15 CB-NK.

### A. General Embodiments of BCMA-Targeting CARs

The present disclosure provides for cells (particularly NK cells) that harbor a vector that encodes at least one CAR, and the CAR may be first generation, second generation, or third or a subsequent generation, for example. The CAR may or may not be bispecific for two or more different antigens, one of which is BCMA. The CAR may comprise one or more co-stimulatory domains. Each co-stimulatory domain may comprise the costimulatory domain of any one or more of, for example, members of the TNFR superfamily, CD28, CD137 (4-1BB), CD134 (OX40), DAP10, DAP12, CD27, CD2, CD5, ICAM-1, LFA-1 (CD11a/CD18), Lck, TNFR-I, TNFR-II, Fas, CD30, CD40 or combinations thereof, for example. In specific embodiments, the CAR comprises CD3zeta. In certain embodiments, the CAR lacks one or more specific costimulatory domains; for example, the CAR may lack 4-1BB.

In a specific embodiment, the CAR comprises DAP12 as a costimulatory domain, and in certain aspects the CAR polypeptide comprises a particular DAP12 amino acid sequence or is encoded by a particular DAP12 nucleic acid sequence. Examples are as follows:

An example of a DAP12 amino acid sequence:

(SEQ ID NO: 1)
MGGLEPCSRLLLLPLLLAVSGLRPVQAQAQSDCSCSTVSPGVLAGIVMGD
LVLTVLIALAVYFLGRLVPRGRGAAEAATRKQRITETESPYQELQGQRSD
VYSDLNTQRPYYK

An example of a DAP12 nucleic acid sequence:

(SEQ ID NO: 2)
ATGGGGGGACTTGAACCCTGCAGCAGGCTCCTGCTCCTGCCTCTCCTGCT
GGCTGTAAGTGGTCTCCGTCCTGTCCAGGCCCAGGCCCAGAGCGATTGCA
GTTGCTCTACGGTGAGCCCGGGCGTGCTGGCAGGGATCGTGATGGGAGAC
CTGGTGCTGACAGTGCTCATTGCCCTGGCCGTGTACTTCCTGGGCCGGCT
GGTCCCTCGGGGGCGAGGGGCTGCGGAGGCAGCGACCCGGAAACAGCGTA
TCACTGAGACCGAGTCGCCTTATCAGGAGCTCCAGGGTCAGAGGTCGGAT
GTCTACAGCGACCTCAACACACAGAGGCCGTATTACAAATGA

In a specific embodiment, the CAR comprises at least CD28 as a costimulatory domain, and in certain aspects the BCMA-targeting CAR polypeptide comprises a particular CD28 amino acid sequence or is encoded by a particular CD28 nucleic acid sequence. Examples are as follows:

An example of a CD28 amino acid sequence including CD28 transmembrane domain and CD28 intracellular domain (but no CD8a or CD3z sequences):

(SEQ ID NO: 3)
VLVVVGGVLACYSLLVTVAFIIFWVRSKRSRLLHSDYMNMTPRRPGPTRK
HYQPYAPPRDFAAYRSR

An example of a CD28 nucleic acid sequence:

(SEQ ID NO: 4)
GTGCTGGTGGTGGTTGGTGGAGTCCTGGCTTGCTATAGCTTGCTAGTAAC
AGTGGCCTTTATTATTTTCTGGGTGAGGAGTAAGAGGAGCAGGCTCCTGC
ACAGTGACTACATGAACATGACTCCCCGCCGCCCCGGGCCCACCCGCAAG
CATTACCAGCCCTATGCCCCACCACGCGACTTCGCAGCCTATCGCT

In particular embodiments, the CAR polypeptide comprises an extracellular spacer domain (that may also be referred to as a hinge) that links the antigen binding domain and the transmembrane domain. Extracellular spacer domains may include, but are not limited to, Fc fragments of antibodies or fragments or derivatives thereof, hinge regions of antibodies or fragments or derivatives thereof, CH2 regions of antibodies, CH3 regions antibodies, artificial spacer sequences or combinations thereof. Examples of extracellular spacer domains include but are not limited to CD8-alpha hinge, CD28 hinge, artificial spacers made of polypeptides such as Gly3, or CH1, CH2, and/or CH3 domains of IgGs (such as human IgG1 or IgG4). In specific cases, the extracellular spacer domain may comprise (i) a hinge, CH2 and CH3 regions of IgG4, (ii) a hinge region of IgG4, (iii) a hinge and CH2 of IgG4, (iv) a hinge region of CD8-alpha, (v) a hinge region of CD28, (vi) a hinge, CH2 and CH3 regions of IgG1, (vii) a hinge region of IgG1 or (viii) a hinge and CH2 of IgG1 or a combination thereof.

In specific embodiments, the hinge is from IgG1 and in certain aspects the CAR polypeptide comprises a particular IgG1 hinge amino acid sequence or is encoded by a particular IgG1 hinge nucleic acid sequence. Examples are as follows:

IgG1 hinge amino acid sequence:

(SEQ ID NO: 5)
SYVTVSSQDPAEPKSPDKTHTCPPCPAPELLGGPSVFLFPPKPKDTLMI
SRTPEVTCVVVDVSHEDPEVKFNWYVDGVEVHNAKTKPREEQYNSTYRV
VSVLTVLHQDWLNGKEYKCKVSNKALPAPIEKTISKAKGQPREPQVYTL
PPSRDELTKNQVSLTCLVKGFYPSDIAVEWESNGQPENNYKTTPPVLDS
DGSFFLYSKLTVDKSRWQQGNVFSCSVMHEALHNHYTQKSLSLSPGKKD
PK

IgG1 hinge nucleic acid sequence:

(SEQ ID NO: 6)
GTACGTCACTGTCTCTTCACAGGATCCCGCCGAGCCCAAATCTCCTGAC
AAAACTCACACATGCCCACCGTGCCCAGCACCTGAACTCCTGGGGGGAC
CGTCAGTCTTCCTCTTCCCCCCAAAACCCAAGGACACCCTCATGATCTC
CCGGACCCCTGAGGTCACATGCGTGGTGGTGGACGTGAGCCACGAAGAC
CCTGAGGTCAAGTTCAACTGGTACGTGGACGGCGTGGAGGTGCATAATG
CCAAGACAAAGCCGCGGGAGGAGCAGTACAACAGCACGTACCGTGTGGT
CAGCGTCCTCACCGTCCTGCACCAGGACTGGCTGAATGGCAAGGAGTAC
AAGTGCAAGGTCTCCAACAAAGCCCTCCCAGCCCCCATCGAGAAAACCA
TCTCCAAAGCCAAAGGGCAGCCCCGAGAACCACAGGTGTACACCCTGCC
CCCATCCCGGGATGAGCTGACCAAGAACCAGGTCAGCCTGACCTGCCTG
GTCAAAGGCTTCTATCCCAGCGACATCGCCGTGGAGTGGGAGAGCAATG
GGCAACCGGAGAACAACTACAAGACCACGCCTCCCGTGCTGGACTCCGA
CGGCTCCTTCTTCCTCTACAGCAAGCTCACCGTGGACAAGAGCAGGTGG
CAGCAGGGGAACGTCTTCTCATGCTCCGTGATGCATGAGGCTCTGCACA
ACCACTACACGCAGAAGAGCCTCTCCCTGTCTCCGGGTAAAAAAGATC

A particular linker that links the VH and VL chains may be utilized. One example of a linker amino acid sequence is as follows: G G G G S G G G G S G G G G S G G G G S (SEQ ID NO:68).

One example of a linker nucleic acid sequence is as follows:

(SEQ ID NO: 69)
GGTGGTGGTGGTTCTGGTGGTGGTGGTTCTGGCGGCGGCGGCTCCGGTG
GTGGTGGATCC

One example of an IgG1 hinge amino acid sequence is as follows (and may differ from SEQ ID NO:50 only in cloning artifact(s)):

(SEQ ID NO: 70)
RTVTVSSQDPAEPKSPDKTHTCPPCPAPELLGGPSVFLFPP
KPKDTLMISRTPEVTCVVVDVSHEDPEVKFNWYVDGVEVHNA
KTKPREEQYNSTYRVVSVLTVLHQDWLNGKEYKCKVSNKALP
APIEKTISKAKGQPREPQVYTLPPSRDELTKNQVSLTCLVKGFY
PSDIAVEWESNGQPENNYKTTPPVLDSDGSFFLYSKLTVDKSR
WQQGNVFSCSVMHEALHNHYTQKSLSLSPGKKDPK

One example of an IgG1 hinge nucleic acid sequence is as follows (and may differ from SEQ ID NO:6 with respect to cloning artifact(s)):

(SEQ ID NO: 71)
CGTACGGTCACTGTCTCTTCACAGGATCCCGCCGAGCCCAAATCTCCTG
ACAAAACTCACACATGCCCACCGTGCCCAGCACCTGAACTCCTGGGGGG
ACCGTCAGTCTTCCTCTTCCCCCCAAAACCCAAGGACACCCTCATGATC
TCCCGGACCCCTGAGGTCACATGCGTGGTGGTGGACGTGAGCCACGAAG
ACCCTGAGGTCAAGTTCAACTGGTACGTGGACGGCGTGGAGGTGCATAA
TGCCAAGACAAAGCCGCGGGAGGAGCAGTACAACAGCACGTACCGTGTG
GTCAGCGTCCTCACCGTCCTGCACCAGGACTGGCTGAATGGCAAGGAGT
ACAAGTGCAAGGTCTCCAACAAAGCCCTCCCAGCCCCCATCGAGAAAAC
CATCTCCAAAGCCAAAGGGCAGCCCCGAGAACCACAGGTGTACACCCTG
CCCCCATCCCGGGATGAGCTGACCAAGAACCAGGTCAGCCTGACCTGCC
TGGTCAAAGGCTTCTATCCCAGCGACATCGCCGTGGAGTGGGAGAGCAA
TGGGCAACCGGAGAACAACTACAAGACCACGCCTCCCGTGCTGGACTCC
GACGGCTCCTTCTTCCTCTACAGCAAGCTCACCGTGGACAAGAGCAGGT
GGCAGCAGGGGAACGTCTTCTCATGCTCCGTGATGCATGAGGCTCTGCA
CAACCACTACACGCAGAAGAGCCTCTCCCTGTCTCCGGGTAAAAAAGAT
CCCAAA

One example of a CD28 costimulatory domain amino acid sequence is as follows:

(SEQ ID NO: 72)
KFWVLVVVGGVLACYSLLVTVAFIIFWVRSKRSRLLHSD
YMNMTPRRPGPTRKHYQPYAPPRDFAAYRSRV

One example of a CD28 costimulatory domain nucleic acid sequence is as follows:

(SEQ ID NO: 73)
AAATTTTGGGTGCTGGTGGTGGTTGGTGGAGTCCTGGCTTGCTATAGCTT
GCTAGTAACAGTGGCCTTTATTATTTTCTGGGTGAGGAGTAAGAGGAGCA
GGCTCCTGCACAGTGACTACATGAACATGACTCCCCGCCGCCCCGGGCCC
ACCCGCAAGCATTACCAGCCCTATGCCCCACCACGCGACTTCGCAGCCTA
TCGCTCACGC

An example of CD8a signal peptide amino acid sequence is as follows:

(SEQ ID NO: 74)
MALPVTALLLPLALLLHAARP

An example of CD8a signal peptide nucleic acid sequence is as follows:

(SEQ ID NO: 75)
ATGGCCCTGCCTGTGACAGCTCTGCTCCTCCCTCTGGCCCTGCTGCTCC
ATGCCGCCAGACCC

One example of GMCSF-R signal peptide amino acid sequence is as follows:

(SEQ ID NO: 76)
MLLLVTSLLLCELPHPAFLLIP

One example of GMCSF-R signal peptide nucleic acid sequence is as follows:

(SEQ ID NO: 77)
ATGCTTCTCCTGGTGACAAGCCTTCTGCTCTGTGAGTTACCACACCCAG
CATTCCTCCTGATCCCA

One example of a CD3 zeta amino acid sequence is as follows:

(SEQ ID NO: 78)
RVKFSRSADAPAYQQGQNQLYNELNLGRREEYDVLDKRR
GRDPEMGGKPRRKNPQEGLYNELQKDKMAEAYSEIGMKGERR
RGKGHDGLYQGLSTATKDTYDALHMQALPPRG

One example of a CD3 zeta nucleic acid sequence is as follows:

(SEQ ID NO: 79)
CGCGTGAAGTTCAGCAGGAGCGCAGACGCCCCCGCGTACCAGCAGGGCC
AGAACCAGCTCTATAACGAGCTCAATCTAGGACGAAGAGAGGAGTACGA
TGTTTTGGACAAAAGACGTGGCCGGGACCCTGAGATGGGGGGAAAGCCG
AGAAGGAAGAACCCTCAGGAAGGCCTGTACAATGAACTGCAGAAAGATA
AGATGGCGGAGGCCTACAGTGAGATTGGGATGAAAGGCGAGCGCCGGAG
GGGCAAGGGGCACGATGGCCTTTACCAGGGTCTCAGTACAGCCACCAAG
GACACCTACGACGCCCTTCACATGCAGGCCCTGCCCCCTCGCGGA

One example of an IL-15 amino acid sequence is as follows:

(SEQ ID NO: 80)
RISKPHLRSISIQCYLCLLLNSHFLTEAGIHVFILGCFSAGL
PKTEANWVNVISDLKKIEDLIQSMHIDATLYTESDVHPSCKVT
AMKCFLLELQVISLESGDASIHDTVENLIILANNSLSSNGNVTE
SGCKECEELEEKNIKEFLQSFVHIVQMFINTS

One example of an IL-15 nucleic acid sequence is as follows:

(SEQ ID NO: 81)
CGCATTAGCAAGCCCCACCTGCGGAGCATCAGCATCCAGTGCTACCTGT
GCCTGCTGCTGAACAGCCACTTCCTGACCGAGGCCGGCATCCACGTGTT
CATCCTGGGCTGCTTCAGCGCCGGACTGCCCAAGACCGAGGCCAACTGG
GTGAACGTGATCAGCGACCTGAAGAAGATCGAGGACCTGATCCAGAGCA
TGCACATCGACGCCACCCTGTACACCGAGAGCGACGTGCACCCCAGCTG
CAAGGTGACCGCCATGAAGTGCTTTCTGCTGGAACTGCAGGTGATCAGC
CTGGAAAGCGGCGACGCCAGCATCCACGACACCGTGGAGAACCTGATCA
TCCTGGCCAACAACAGCCTGAGCAGCAACGGCAACGTGACCGAGAGCGG
CTGCAAAGAGTGCGAGGAACTGGAAGAGAAGAACATCAAAGAGTTTCTG
CAGAGCTTCGTGCACATCGTGCAGATGTTCATCAACACCAGC

### B. Specific Examples of CAR Constructs

In specific examples of CAR constructs encompassed herein, there are particular (but interchangeable) selections for a variety of elements of the CAR and/or the vector itself.

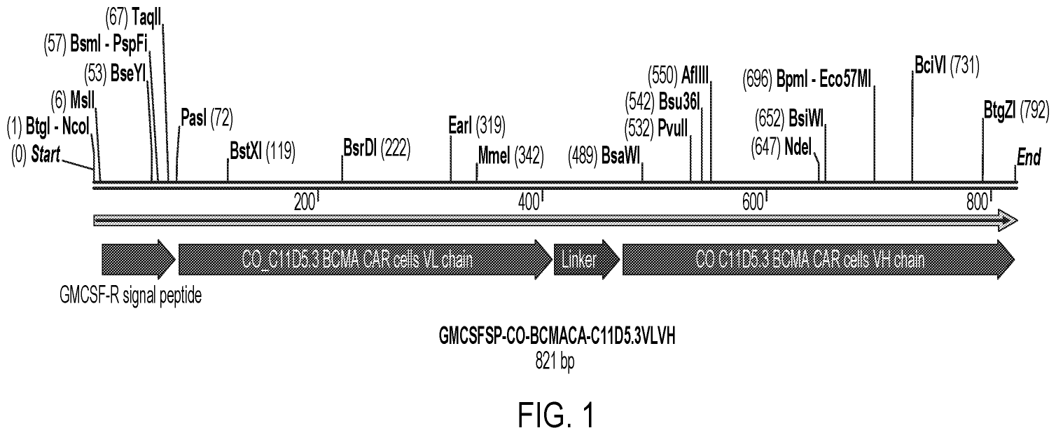

One example of a particular vector construct including a BCMA-targeting CAR is illustrated in FIG. 1. This vector includes the BCMA-targeting CAR having the granulocyte-macrophage colony-stimulating factor receptor signaling peptide (GMCSFRsp) as part of a CAR that includes codon optimized (co) versions of the VH chain and VL chain of C11D5.3 antibody (as one example) separated by a linker of any kind. The VL chain is upstream of the VH chain in a 5′ to 3′ direction, in this embodiment. The particular CAR in FIG. 1 also uses the CH2CH3 domain of IgG1 as the hinge and CD28 as a costimulatory domain, in addition to CD3zeta. The vector comprises a cytokine, such as IL-15, and the cytokine becomes a separate polypeptide from the CAR with the utilization of the 2A element that separates them.

In some embodiments of BCMA-targeting CARs, the vector expressing the CAR may also express one or more suicide genes. As one example, a TNFa mutant may be utilized as a suicide gene. In the examples of the vectors in the figures herein, the TNFa mutant that was utilized was del Ala-1 to Val 13 (14aa del) CKI mut 5 aa mutant (see elsewhere herein; SEQ ID NO:37), although any other suicide genes, including other mutant TNFalphas, may be utilized.

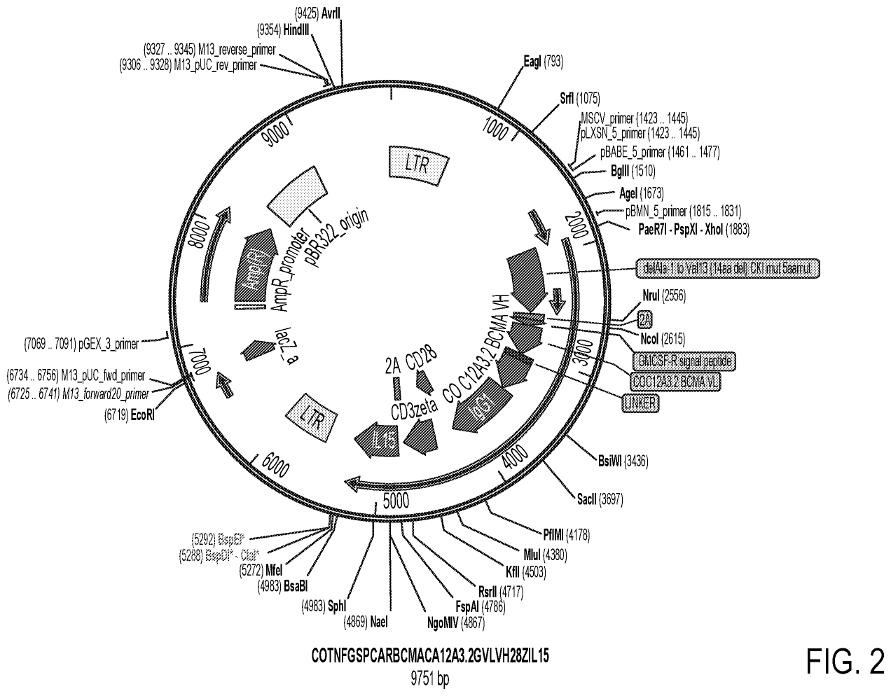

One example of a BCMA-targeting CAR is illustrated in FIG. 2, and the vector encompasses an example of a TNFa mutant and a BCMA-targeting CAR that employs a codon optimized version of the C12A3.2 antibody. In particular embodiments, the TNFa mutant-2A-GMCSFRspcoC12A3.2 BCMAVLVH28Z-2A-IL15 may be provided in a vector and include (1) a TNFa mutant suicide gene that upon processing of the 2A element becomes a separate polypeptide from (2) a BCMA CAR including the granulocyte-macrophage colony-stimulating factor receptor signaling peptide (GMCSFRsp) and the co C12A3.2 antibody; and that upon processing of the 2A element also becomes a separate polypeptide from (3) a cytokine. In this particular embodiment, the nature of the intervening 2A sequences allows for ultimate production of separate polypeptides for the TNFa mutant, the BCMA-targeting CAR, and the cytokine. In FIG. 2, the VL chain of C12A3.2 is upstream of the VH chain in a 5′ to 3′ direction, in this particular example. This specific CAR also utilizes CD28 and CD3zeta.

Certain constructs utilize suicide genes, including TNFa mutants in some cases. In any specific example of vector constructs utilizing a TNFa mutant, an example of a nucleotide sequence of one example of a TNFa mutant as a suicide gene is as follows (and this suicide gene and any others may be used in other specific constructs):

(SEQ ID NO: 7)
ATGAGCACTGAAATGCATCCCGGAAGGGGGTCCTGGCACGAGGAGGCGC
TCCCCAAGAAGACAGGGGGGCCCCAGGGCTCCAGGCGGTGCTTGTTCCT
CAGCCTCTTCTCCTTCCTGATCGTGGCAGGCGCCACCACGCTCTTCTTC
CTGCTGCACTTTGGAGTGATCGGCCCCCAGAGGGAAGAGTTCCCCAGGG
ACCTCTCTCTAATCAGCCCTCTGCAGGCAGCCCATGTTGTAGCAAACCC
TCAAGCTGAGGGGCAGCTCCAGTGGCTGAACCGCCGGGCCAATGCCCTC
CTGGCCAATGGCGTGGAGCTGAGAGATAACCAGCTGGTTGTGCCATCAG
AGGGCCTGTACCTCATCTACTCCCAGGTCCTCTTCAAGGGCCAAGGCTG
CCCCTCCACCCATGTGCTCCTCACCCACACCATCAGCCGCATCGCCGTC
TCCCACCAGACCAAGGTCAACCTCCTCTTCGCCATCAAGAGCCCCTGCC
AGAGGGAGACCCCAGAGGGGGCTGAGGCTAAGCCCTGGTATGAGCCCAT
CTATCTGGGAGGGGTCTTCCAGCTGGAGAAGGGTGACCGACTCATCGCT
GAGATCAATCGGCCCGACTATCTCTACTTTGCCGAGTATGGGCAGGTCT
ACTTTGGGATCATTGCCCTGTCG

An example of a nucleotide sequence of BCMA CAR that utilizes the coC11D5.3 antibody instead of the co C12A3.2 antibody is as follows (and may be referred to as (GMCSFRspcoC11D5.3 BCMAVLVH):

(SEQ ID NO: 8)
ATGCTTCTCCTGGTGACAAGCCTTCTGCTCTGTGAGTTACCACACCCAG
CATTCCTCCTGATCCCAGGGGACATTGTTTTGACCCAATCACCTCCCTC
TCTCGCCATGTCCTTGGGTAAACGGGCAACAATCTCCTGTAGAGCTTCC
GAAAGTGTAACAATTCTTGGAAGCCACCTCATACATTGGTATCAGCAAA
AGCCGGGGCAGCCCCCTACATTGCTCATTCAGTTGGCTTCAAATGTCCA
GACGGGTGTACCAGCGAGATTCTCAGGGAGTGGCTCCCGAACGGATTTC
ACACTGACGATTGATCCCGTCGAAGAGGACGATGTCGCAGTTTATTATT
GCCTCCAAAGTCGGACAATTCCGAGGACTTTTGGAGGCGGAACAAAATT
GGAAATCAAAGGGGGTGGAGGTTCTGGCGGAGGGGGCAGCGGTGGTGGA
GGAAGTGGGGGCGGTGGGAGTCAAATCCAGCTCGTCCAATCCGGTCCAG
AGTTGAAGAAACCCGGCGAGACGGTAAAAATCAGCTGTAAAGCCTCAGG
TTACACGTTTACGGACTATAGCATTAATTGGGTTAAGAGGGCTCCGGGG
AAGGGGCTCAAATGGATGGGCTGGATAAACACAGAGACGAGAGAGCCCG
CATATGCGTTCGACTTTAGAGGTCGATTCGCTTTCAGTCTTGAAACCTC
TGCTTCTACCGCGTATCTCCAGATAAACAACCTGAAATATGAGGATACA
GCAACTTATTTTTGCGCTCTCGATTACAGCTATGCGATGGATTATTGGG
GACAAGGAACTTCCGTGACTGTGTCAAGC

A polypeptide sequence of BCMA CAR (GMCSFRspcoC11D5.3 BCMAVLVH) utilizing the C11D5.3 antibody is as follows:

(SEQ ID NO: 9)
MLLLVTSLLLCELPHPAFLLIPDIVLTQSPPSLAMSLGKRATISCRASE
SVTILGSHLIHWYQQKPGQPPTLLIQLASNVQTGVPARFSGSGSRTDFT
LTIDPVEEDDVAVYYCLQSRTIPRTFGGGTKLEIKGGGGSGGGGSGGGG
SGGGGSQIQLVQSGPELKKPGETVKISCKASGYTFTDYSINWVKRAPGK
GLKWMGWINTETREPAYAFDFRGRFAFSLETSASTAYLQINNLKYEDTA
TYFCALDYSYAMDYWGQGTSVTVSS

A nucleotide sequence of one example of a BCMA CAR (GMCSFRspcoC12A3.2 BCMAVLVH) utilizing the codon-optimized C12A3.2 antibody (see FIG. 2) is as follows:

(SEQ ID NO: 10)
TGCTTCTCCTGGTGACAAGCCTTCTGCTCTGTGAGTTACCACACCCAGC
ATTCCTCCTGATCCCAGGGGACATCGTGCTGACCCAGAGCCCCCCCAGC
CTGGCCATGTCTCTGGGCAAGAGAGCCACCATCAGCTGCCGGGCCAGCG
AGAGCGTGACCATCCTGGGCAGCCACCTGATCTACTGGTATCAGCAGAA
GCCTGGCCAGCCCCCCACCCTGCTGATCCAGCTGGCTAGCAATGTGCAG
ACCGGCGTGCCCGCCAGATTCAGCGGCAGCGGCAGCAGAACCGACTTCA
CCCTGACCATCGACCCCGTGGAAGAGGACGACGTGGCCGTGTACTACTG
CCTGCAGAGCCGGACCATCCCCCGGACCTTTGGCGGAGGAACAAAGCTG
GAAATCAAGGGTGGTGGTGGTTCTGGTGGTGGTGGTTCTGGCGGCGGCG
GCTCCGGTGGTGGTGGATCCCAGATTCAGCTGGTGCAGAGCGGCCCTGA
GCTGAAGAAACCCGGCGAGACAGTGAAGATCAGCTGCAAGGCCTCCGGC
TACACCTTCCGGCACTACAGCATGAACTGGGTGAAACAGGCCCCTGGCA
AGGGCCTGAAGTGGATGGGCCGGATCAACACCGAGAGCGGCGTGCCCAT
CTACGCCGACGACTTCAAGGGCAGATTCGCCTTCAGCGTGGAAACCAGC
GCCAGCACCGCCTACCTGGTGATCAACAACCTGAAGGACGAGGATACCG
CCAGCTACTTCTGCAGCAACGACTACCTGTACAGCCTGGACTTCTGGGG
CCAGGGCACCGCCCTGACCGTGTCCAGC

A polypeptide of BCMA CAR (GMCSFRspcoC12A3.2 BCMAVLVH) utilizing the codon-optimized C12A3.2 antibody (see FIG. 2) is as follows:

(SEQ ID NO: 11)
MLLLVTSLLLCELPHPAFLLIPGDIVLTQSPPSLAMSLGKRATISCRAS
ESVTILGSHLIYWYQQKPGQPPTLLIQLASNVQTGVPARFSGSGSRTDF
TLTIDPVEEDDVAVYYCLQSRTIPRTFGGGTKLEIKGGGGSGGGGSGGG
GSGGGGSQIQLVQSGPELKKPGETVKISCKASGYTFRHYSMNWVKQAPG
KGLKWMGRINTESGVPIYADDFKGRFAFSVETSASTAYLVINNLKDEDT
ASYFCSNDYLYSLDFWGQGTALTVSS

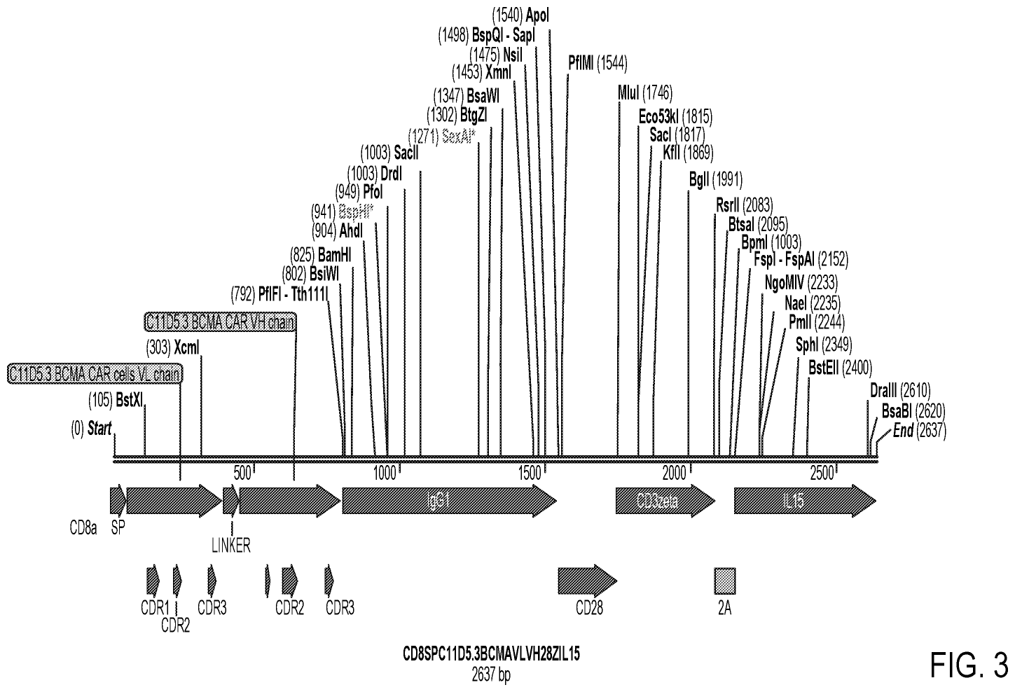

In some examples of vector constructs, a signal peptide from CD8a is utilized instead of a signal peptide from GMCSFR. In the example of the vector in FIG. 3, a CD8a signal peptide is employed with C11D5.3 BCMA VL chain linked by a linker to C11D5.3 BCMA VH chain (and the VL chain is upstream of the VH chain in a 5′ to 3′ direction in this example), the IgG1 hinge, CD28, and CD3zeta, followed by IL-15 (separated by a 2A element). In such cases, a suicide gene including mutant TNFa may or may not be utilized.

An example of a nucleotide sequence for CD8spC11D53VLVH is as follows:

(SEQ ID NO: 12)
ATGGCCCTGCCTGTGACAGCTCTGCTCCTCCCTCTGGCCCTGCTGCTCC
ATGCCGCCAGACCCGACATCGTGCTGACCCAGAGCCCCCCCAGCCTGGC
CATGTCTCTGGGCAAGAGAGCCACCATCAGCTGCCGGGCCAGCGAGAGC
GTGACCATCCTGGGCAGCCACCTGATCCACTGGTATCAGCAGAAGCCCG
GCCAGCCCCCCACCCTGCTGATCCAGCTCGCCAGCAATGTGCAGACCGG
CGTGCCCGCCAGATTCAGCGGCAGCGGCAGCAGAACCGACTTCACCCTG
ACCATCGACCCCGTGGAAGAGGACGACGTGGCCGTGTACTACTGCCTGC
AGAGCCGGACCATCCCCCGGACCTTTGGCGGAGGCACCAAACTGGAAAT
CAAGGGCAGCACCAGCGGCTCCGGCAAGCCTGGCTCTGGCGAGGGCAGC
ACAAAGGGACAGATTCAGCTGGTGCAGAGCGGCCCTGAGCTGAAGAAAC
CCGGCGAGACAGTGAAGATCAGCTGCAAGGCCTCCGGCTACACCTTCAC
CGACTACAGCATCAACTGGGTGAAAAGAGCCCCTGGCAAGGGCCTGAAG
TGGATGGGCTGGATCAACACCGAGACAAGAGAGCCCGCCTACGCCTACG
ACTTCCGGGGCAGATTCGCCTTCAGCCTGGAAACCAGCGCCAGCACCGC
CTACCTGCAGATCAACAACCTGAAGTACGAGGACACCGCCACCTACTTT
TGCGCCCTGGACTACAGCTACGCTATGGACTACTGGGGCCAGGGCACCA
GCGTGACCGTGTCCAGC

An example of a polypeptide sequence for CD8spC11D53VLVH is as follows:

(SEQ ID NO: 13)
MALPVTALLLPLALLLHAARPDIVLTQSPPSLAMSLGKRATISCRASES
VTILGSHLIHWYQQKPGQPPTLLIQLASNVQTGVPARFSGSGSRTDFTL
TIDPVEEDDVAVYYCLQSRTIPRTFGGGTKLEIKGSTSGSGKPGSGEGS
TKGQIQLVQSGPELKKPGETVKISCKASGYTFTDYSINWVKRAPGKGLK
WMGWINTETREPAYAYDFRGRFAFSLETSASTAYLQINNLKYEDTATYF
CALDYSYAMDYWGQGTSVTVSS

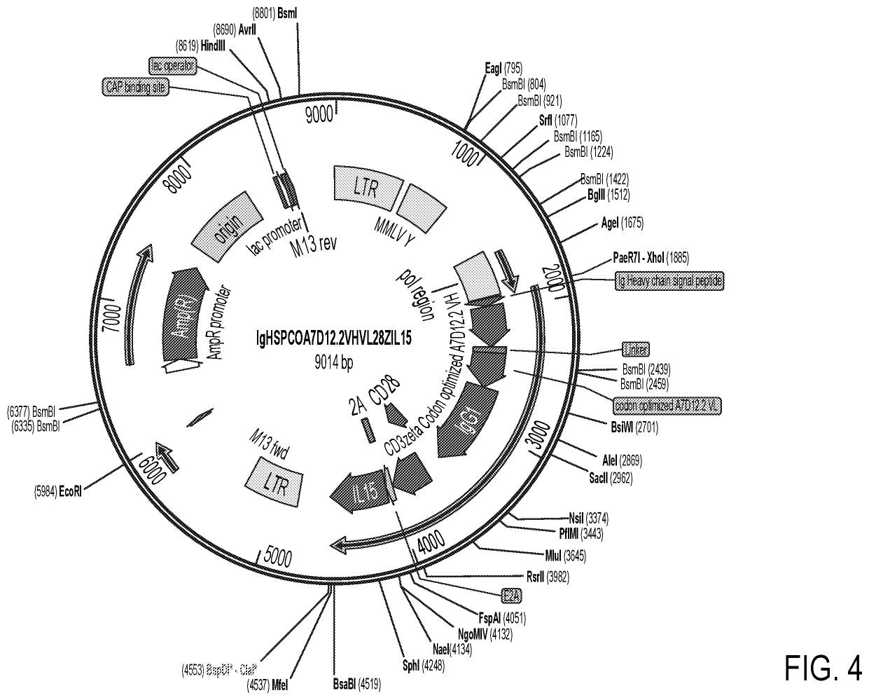

One example of an expression construct utilizing a TNFa mutant and the C12A3.2 antibody is exemplified in FIG. 4. In such an example, a TNFa mutant is separated by a 2A element from the BCMA-targeting CAR that includes GMCSF-R signal peptide, the C12A3.2 VL chain upstream of, but linked through a linker to, the C12A3.2 VH chain, and the CAR also includes the IgG1 hinge, CD28, and CD3zeta. A further 2A element separates the BCMA-targeting CAR from IL-15.

An example of a nucleotide sequence expressing TNFamut-CD8spC12A3.2.BCMAVLVH is as follows:

(SEQ ID NO: 14)
GGATGGCCCTGCCTGTGACAGCTCTGCTGCTGCCCCTGGCCCTGCTGCT
CCATGCCGCCAGACCCGACATCGTGCTGACCCAGAGCCCCCCCAGCCTG
GCCATGTCTCTGGGCAAGAGAGCCACCATCAGCTGCCGGGCCAGCGAGA
GCGTGACCATCCTGGGCAGCCACCTGATCTACTGGTATCAGCAGAAGCC
TGGCCAGCCCCCCACCCTGCTGATCCAGCTGGCTAGCAATGTGCAGACC
GGCGTGCCCGCCAGATTCAGCGGCAGCGGCAGCAGAACCGACTTCACCC
TGACCATCGACCCCGTGGAAGAGGACGACGTGGCCGTGTACTACTGCCT
GCAGAGCCGGACCATCCCCCGGACCTTTGGCGGAGGAACAAAGCTGGAA
ATCAAGGGCAGCACCAGCGGCTCCGGCAAGCCTGGCTCTGGCGAGGGCA
GCACAAAGGGACAGATTCAGCTGGTGCAGAGCGGCCCTGAGCTGAAGAA
ACCCGGCGAGACAGTGAAGATCAGCTGCAAGGCCTCCGGCTACACCTTC
CGGCACTACAGCATGAACTGGGTGAAACAGGCCCCTGGCAAGGGCCTGA
AGTGGATGGGCCGGATCAACACCGAGAGCGGCGTGCCCATCTACGCCGA
CGACTTCAAGGGCAGATTCGCCTTCAGCGTGGAAACCAGCGCCAGCACC
GCCTACCTGGTGATCAACAACCTGAAGGACGAGGATACCGCCAGCTACT
TCTGCAGCAACGACTACCTGTACAGCCTGGACTTCTGGGGCCAGGGCAC
CGCCCTGACCGTGTCCAGC

An example of a polypeptide sequence for TNFamut-CD8spC12A3.2.BCMAVLVH is as follows:

(SEQ ID NO: 15)
DIVLTQSPPSLAMSLGKRATISCRASESVTILGSHLIYWYQQKPGQPPT
LLIQLASNVQTGVPARFSGSGSRTDFTLTIDPVEEDDVAVYYCLQSRTI
PRTFGGGTKLEIKGGGGSGGGGSGGGGSQIQLVQSGPELKKPGETVKIS
CKASGYTFRHYSMNWVKQAPGKGLKWMGRINTESGVPIYADDFKGRFAF
SVETSASTAYLVINNLKDEDTASYFCSNDYLYSLDFWGQGTALTVSS

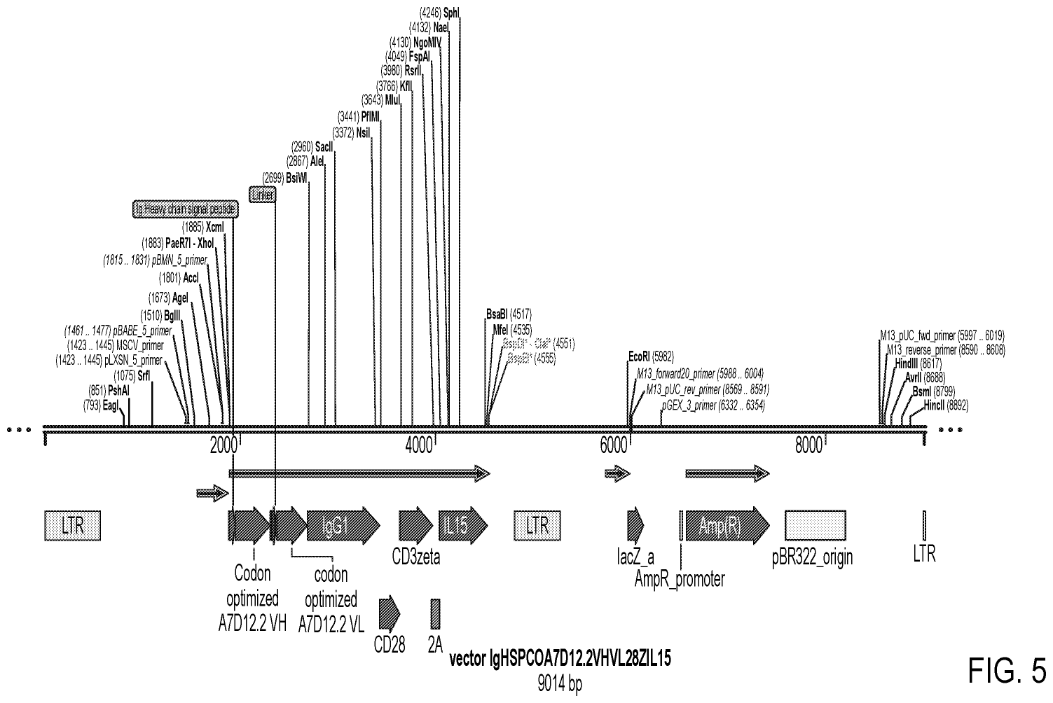

FIG. 5 provides an example of a vector comprising an expression construct expressing IgHspCOA7D12.2VHVL that includes an Ig heavy chain signal peptide and codon optimized A7D12.2VH and A7D12.2VL, and it also includes the IgG1 hinge, CD28, and CD3zeta. In this example for IgHspCOA7D12.2VHVL, the VH element is upstream of the VL element in a 5′ to 3′ direction. The vector also includes a 2A element that separates IL-15 from the CAR. A suicide gene may or may not be included in the vector, and when a suicide gene is used a 2A element may or may not be the element that separates the CAR from the suicide gene.

An example of a nucleotide sequence for IgHspCOA7D12.2VHVL is as follows:

(SEQ ID NO: 16)
ATGGAGTTTGGGCTGAGCTGGCTTTTTCTTGTGGCTATTTTAAAAGGTG
TCCAGTGCTCTAGACAGATACAGCTCGTCCAATCCGGTCCCGATTTGAA
AAAGCCTGGCGAAACAGTTAAACTGTCATGTAAGGCGAGCGGATACACG
TTTACGAACTTCGGGATGAATTGGGTAAAACAGGCCCCGGGAAAAGGTT
TTAAGTGGATGGCTTGGATAAACACCTACACTGGTGAGTCCTACTTCGC
AGACGATTTCAAAGGGCGGTTCGCGTTTTCAGTAGAGACTTCCGCCACA
ACTGCTTATCTCCAAATAAACAACTTGAAGACCGAGGATACGGCAACCT
ACTTTTGCGCTCGGGGCGAGATTTATTATGGATATGACGGCGGGTTCGC
TTACTGGGGTCAGGGGACGTTGGTTACCGTGTCTGCCGGTGGTGGTGGT
TCTGGTGGTGGTGGTTCTGGCGGCGGCGGCTCCGGTGGTGGTGGATCCG
ACGTGGTGATGACGCAGAGCCACCGATTCATGAGTACCTCTGTAGGCGA
CCGCGTCTCAATTACTTGTCGAGCGTCTCAGGACGTAAATACAGCGGTG
AGCTGGTATCAGCAAAAGCCCGGACAGAGCCCGAAATTGCTGATCTTTT
CAGCCTCATACAGATATACCGGAGTCCCAGACCGCTTTACAGGTTCCGG
TAGTGGCGCGGACTTTACTCTCACAATCAGCTCTGTACAAGCTGAAGAT
TTGGCTGTTTACTATTGTCAGCAGCACTATAGTACGCCCTGGACCTTCG
GGGGCGGTACGAAGTTGGATATTAAG

An example of a polypeptide sequence for IgHspCOA7D12.2VHVL is as follows:

(SEQ ID NO: 17)
MEFGLSWLFLVAILKGVQCSRQIQLVQSGPDLKKPGETVKLSCKASGYT
FTNFGMNWVKQAPGKGFKWMAWINTYTGESYFADDFKGRFAFSVETSAT
TAYLQINNLKTEDTATYFCARGEIYYGYDGGFAYWGQGTLVTVSAGGGG
SGGGGSGGGGSGGGGSDVVMTQSHRFMSTSVGDRVSITCRASQDVNTAV
SWYQQKPGQSPKLLIFSASYRYTGVPDRFTGSGSGADFTLTISSVQAED
LAVYYCQQHYSTPWTFGGGTKLDIK

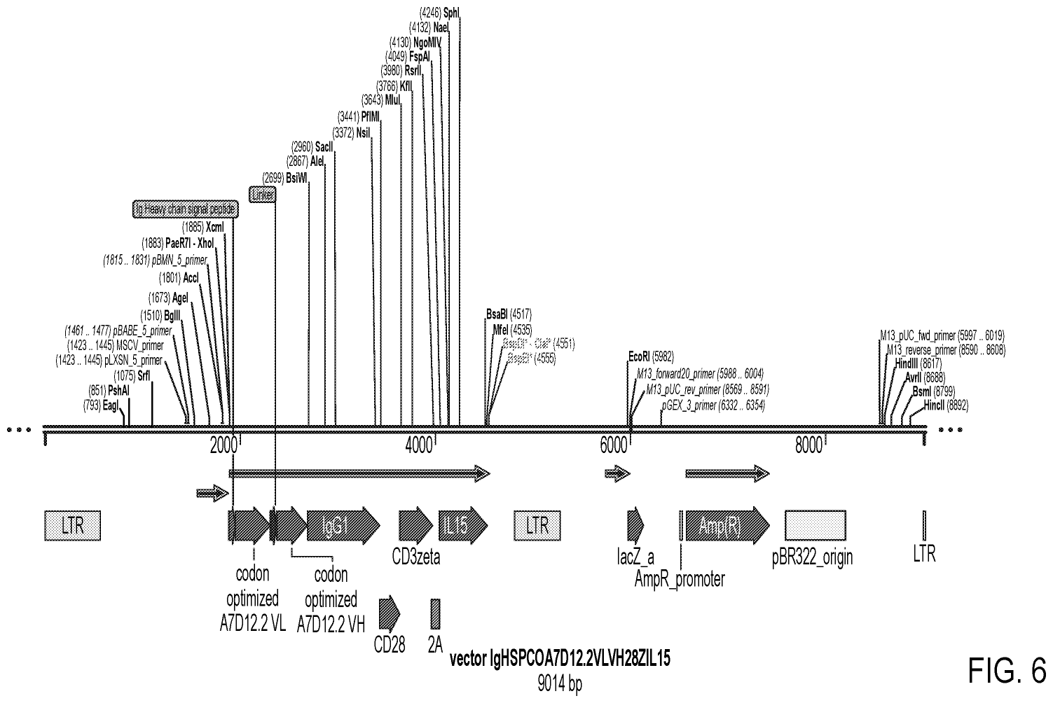

FIG. 6 provides one example of a vector including an expression construct that expresses IgHspCOA7D12.2VLVH. In this example for IgHspCOA7D12.2VLVH, the VL element is upstream of the VH element in a 5′ to 3′ direction. An example of a polynucleotide that encodes IgHspCOA7D12.2VLVH is as follows:

(SEQ ID NO: 18)
ATGGAGTTTGGGCTGAGCTGGCTTTTTCTTGTGGCTATTTTAAAAGGTG
TCCAGTGCTCTAGAGACGTGGTGATGACGCAGAGCCACCGATTCATGAG
TACCTCTGTAGGCGACCGCGTCTCAATTACTTGTCGAGCGTCTCAGGAC
GTAAATACAGCGGTGAGCTGGTATCAGCAAAAGCCCGGACAGAGCCCGA
AATTGCTGATCTTTTCAGCCTCATACAGATATACCGGAGTCCCAGACCG
CTTTACAGGTTCCGGTAGTGGCGCGGACTTTACTCTCACAATCAGCTCT
GTACAAGCTGAAGATTTGGCTGTTTACTATTGTCAGCAGCACTATAGTA
CGCCCTGGACCTTCGGGGGCGGTACGAAGTTGGATATTAAGGGTGGTGG
TGGTTCTGGTGGTGGTGGTTCTGGCGGCGGCGGCTCCGGTGGTGGTGGA
TCCCAGATACAGCTCGTCCAATCCGGTCCCGATTTGAAAAAGCCTGGCG
AAACAGTTAAACTGTCATGTAAGGCGAGCGGATACACGTTTACGAACTT
CGGGATGAATTGGGTAAAACAGGCCCCGGGAAAAGGTTTTAAGTGGATG
GCTTGGATAAACACCTACACTGGTGAGTCCTACTTCGCAGACGATTTCA
AAGGGCGGTTCGCGTTTTCAGTAGAGACTTCCGCCACAACTGCTTATCT
CCAAATAAACAACTTGAAGACCGAGGATACGGCAACCTACTTTTGCGCT
CGGGGCGAGATTTATTATGGATATGACGGCGGGTTCGCTTACTGGGGTC
AGGGGACGTTGGTTACCGTGTCTGCC

An example of a polypeptide for IgHspCOA7D12.2VLVH is as follows:

(SEQ ID NO: 19)
MEFGLSWLFLVAILKGVQCSRDVVMTQSHRFMSTSVGDRVSITCRASQD
VNTAVSWYQQKPGQSPKLLIFSASYRYTGVPDRFTGSGSGADFTLTISS
VQAEDLAVYYCQQHYSTPWTFGGGTKLDIKGGGGSGGGGSGGGGSGGGG
SQIQLVQSGPDLKKPGETVKLSCKASGYTFTNFGMNWVKQAPGKGFKWM
AWINTYTGESYFADDFKGRFAFSVETSATTAYLQINNLKTEDTATYFCA
RGEIYYGYDGGFAYWGQGTLVTVSA

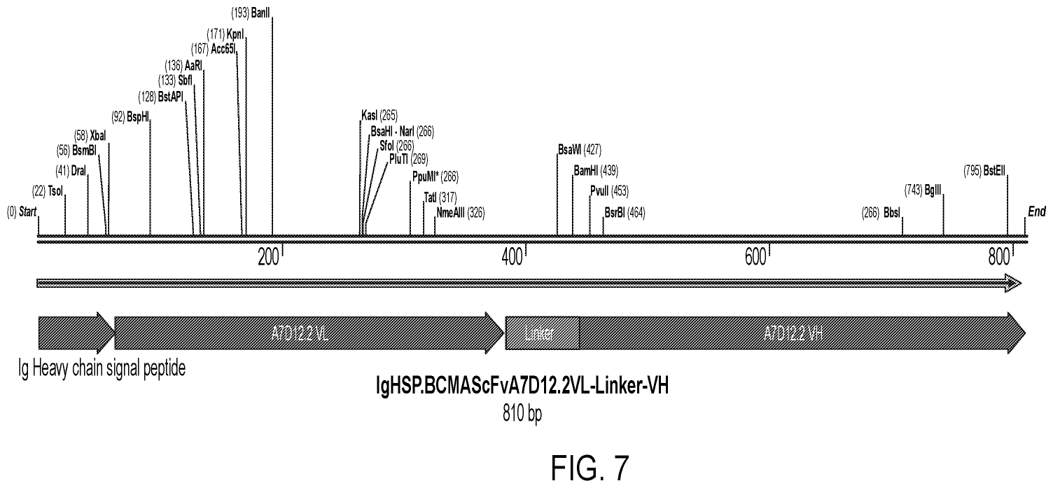

FIG. 7 provides one example of an expression vector that encodes a BCMA-targeting CAR with A7D12.2 VL chain linked in a 5′ to 3′ direction to A7D12.2 VH chain and also including the Ig heavy chain signal peptide.

An example of a polynucleotide that encodes an expression construct for IgHSP.BCMAScFvA7D12.2VL-Linker-VH is as follows:

(SEQ ID NO: 56)
atggagtttgggctgagctggctttttcttgtggctattttaaaaggtgt
ccagtgctctagaGACGTGGTGATGACCCAGAGCCACAGGTTCATGAGCA
CCAGCGTGGGCGACAGGGTGAGCATCACCTGCAGGGCCAGCCAGGACGTG
AACACCGCCGTGAGCTGGTACCAGCAGAAGCCCGGCCAGAGCCCCAAGCT
GCTGATCTTCAGCGCCAGCTACAGGTACACCGGCGTGCCCGACAGGTTCA
CCGGCAGCGGCAGCGGCGCCGACTTCACCCTGACCATCAGCAGCGTGCAG
GCCGAGGACCTGGCCGTGTACTACTGCCAGCAGCACTACAGCACCCCCTG
GACCTTCGGCGGCGGCACCAAGCTGGACATCAAGGGTGGTGGTGGTTCTG
GTGGTGGTGGTTCTGGCGGCGGCGGCTCCGGTGGTGGTGGATCCCAGATC
CAGCTGGTGCAGAGCGGCCCCGACCTGAAGAAGCCCGGCGAGACCGTGAA
GCTGAGCTGCAAGGCCAGCGGCTACACCTTCACCAACTTCGGCATGAACT
GGGTGAAGCAGGCCCCCGGCAAGGGCTTCAAGTGGATGGCCTGGATCAAC
ACCTACACCGGCGAGAGCTACTTCGCCGACGACTTCAAGGGCAGGTTCGC
CTTCAGCGTGGAGACCAGCGCCACCACCGCCTACCTGCAGATCAACAACC
TGAAGACCGAGGACACCGCCACCTACTTCTGCGCCAGGGGCGAGATCTAC
TACGGCTACGACGGCGGCTTCGCCTACTGGGGCCAGGGCACCCTGGTGAC
CGTGAGCGCC

An example of a polypeptide for IgHSP.BCMAScFvA7D12.2VL-Linker-VH is as follows:

(SEQ ID NO: 57)
MEFGLSWLFLVAILKGVQCSRDVVMTQSHRFMSTSVGDRVSITCRASQD
VNTAVSWYQQKPGQSPKLLIFSASYRYTGVPDRFTGSGSGADFTLTISS
VQAEDLAVYYCQQHYSTPWTFGGGTKLDIKGGGGSGGGGSGGGGSGGGG
SQIQLVQSGPDLKKPGETVKLSCKASGYTFTNFGMNWVKQAPGKGFKWM
AWINTYTGESYFADDFKGRFAFSVETSATTAYLQINNLKTEDTATYFCA
RGEIYYGYDGGFAYWGQGTLVTVSA

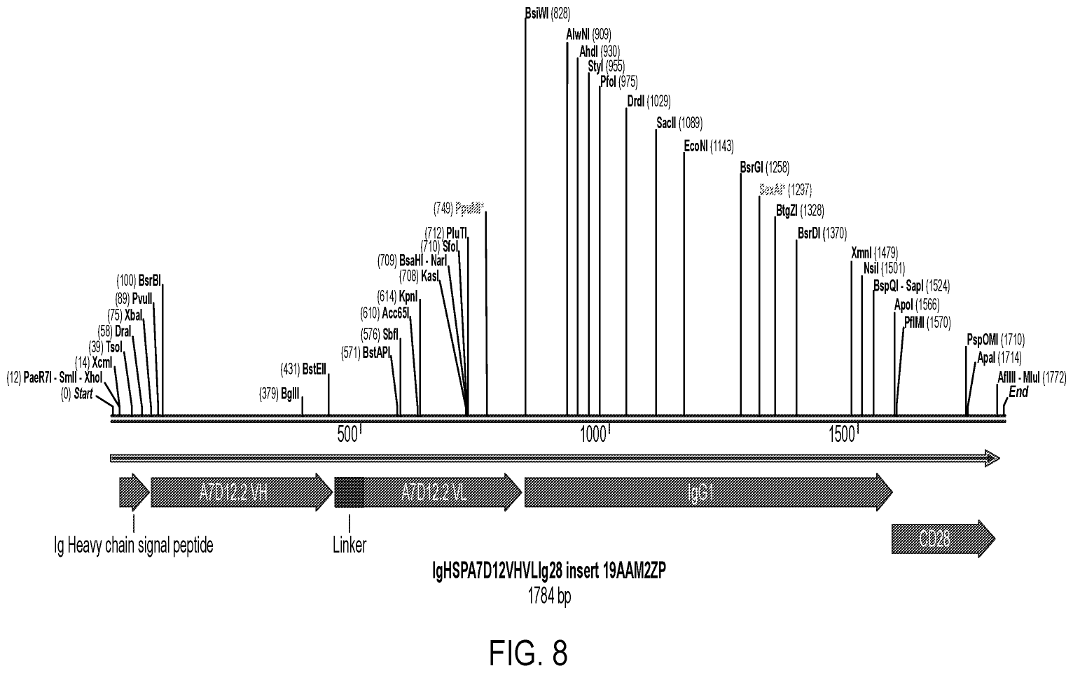

FIG. 8 illustrates an example of an expression vector that encodes a BCMA-targeting CAR with A7D12.2 VH chain linked in a 5′ to 3′ direction to A7D12.2 VL chain and the IgG1 hinge. The CAR utilizes the Ig heavy chain signal peptide and the CD28 costimulatory domain.

An example of a polynucleotide that encodes an expression construct for IgHSPA7D12VHVLIg28 is as follows:

(SEQ ID NO: 58)
AGACTGCCATGCTCGAGATGGAGTTTGGGCTGAGCTGGCTTTTTCTTGTG
GCTATTTTAAAAGGTGTCCAGTGCTCTAGACAGATCCAGCTGGTGCAGAG
CGGCCCCGACCTGAAGAAGCCCGGCGAGACCGTGAAGCTGAGCTGCAAGG
CCAGCGGCTACACCTTCACCAACTTCGGCATGAACTGGGTGAAGCAGGCC
CCCGGCAAGGGCTTCAAGTGGATGGCCTGGATCAACACCTACACCGGCGA
GAGCTACTTCGCCGACGACTTCAAGGGCAGGTTCGCCTTCAGCGTGGAGA
CCAGCGCCACCACCGCCTACCTGCAGATCAACAACCTGAAGACCGAGGAC
ACCGCCACCTACTTCTGCGCCAGGGGCGAGATCTACTACGGCTACGACGG
CGGCTTCGCCTACTGGGGCCAGGGCACCCTGGTGACCGTGAGCGCCGGTG
GTGGTGGTTCTGGTGGTGGTGGTTCTGGCGGCGGCGGCTCCGGTGGTGGT
GGATCCGACGTGGTGATGACCCAGAGCCACAGGTTCATGAGCACCAGCGT
GGGCGACAGGGTGAGCATCACCTGCAGGGCCAGCCAGGACGTGAACACCG
CCGTGAGCTGGTACCAGCAGAAGCCCGGCCAGAGCCCCAAGCTGCTGATC
TTCAGCGCCAGCTACAGGTACACCGGCGTGCCCGACAGGTTCACCGGCAG
CGGCAGCGGCGCCGACTTCACCCTGACCATCAGCAGCGTGCAGGCCGAGG
ACCTGGCCGTGTACTACTGCCAGCAGCACTACAGCACCCCCTGGACCTTC
GGCGGCGGCACCAAGCTGGACATCAAGCGTACGGTCACTGTCTCTTCACA
GGATCCCGCCGAGCCCAAATCTCCTGACAAAACTCACACATGCCCACCGT
GCCCAGCACCTGAACTCCTGGGGGGACCGTCAGTCTTCCTCTTCCCCCCA
AAACCCAAGGACACCCTCATGATCTCCCGGACCCCTGAGGTCACATGCGT
GGTGGTGGACGTGAGCCACGAAGACCCTGAGGTCAAGTTCAACTGGTACG
TGGACGGCGTGGAGGTGCATAATGCCAAGACAAAGCCGCGGGAGGAGCAG
TACAACAGCACGTACCGTGTGGTCAGCGTCCTCACCGTCCTGCACCAGGA
CTGGCTGAATGGCAAGGAGTACAAGTGCAAGGTCTCCAACAAAGCCCTCC
CAGCCCCCATCGAGAAAACCATCTCCAAAGCCAAAGGGCAGCCCCGAGAA
CCACAGGTGTACACCCTGCCCCCATCCCGGGATGAGCTGACCAAGAACCA
GGTCAGCCTGACCTGCCTGGTCAAAGGCTTCTATCCCAGCGACATCGCCG
TGGAGTGGGAGAGCAATGGGCAACCGGAGAACAACTACAAGACCACGCCT
CCCGTGCTGGACTCCGACGGCTCCTTCTTCCTCTACAGCAAGCTCACCGT
GGACAAGAGCAGGTGGCAGCAGGGGAACGTCTTCTCATGCTCCGTGATGC
ATGAGGCTCTGCACAACCACTACACGCAGAAGAGCCTCTCCCTGTCTCCG
GGTAAAAAAGATCCCAAATTTTGGGTGCTGGTGGTGGTTGGTGGAGTCCT
GGCTTGCTATAGCTTGCTAGTAACAGTGGCCTTTATTATTTTCTGGGTGA
GGAGTAAGAGGAGCAGGCTCCTGCACAGTGACTACATGAACATGACTCCC
CGCCGCCCCGGGCCCACCCGCAAGCATTACCAGCCCTATGCCCCACCACG
CGACTTCGCAGCCTATCGCTCACGCGTGAAGTTC

An example of a polypeptide for IgHSPA7D12VHVLIg28 is as follows:

(SEQ ID NO: 59)
TAMLEMEFGLSWLFLVAILKGVQCSRQIQLVQSGPDLKKPGETVKLSCKA
SGYTFTNFGMNWVKQAPGKGFKWMAWINTYTGESYFADDFKGRFAFSVET
SATTAYLQINNLKTEDTATYFCARGEIYYGYDGGFAYWGQGTLVTVSAGG
GGSGGGGSGGGGSGGGGSDVVMTQSHRFMSTSVGDRVSITCRASQDVNTA
VSWYQQKPGQSPKLLIFSASYRYTGVPDRFTGSGSGADFTLTISSVQAED
LAVYYCQQHYSTPWTFGGGTKLDIKRTVTVSSQDPAEPKSPDKTHTCPPC
PAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALP
APIEKTISKAKGQPREPQVYTLPPSRDELTKNQVSLTCLVKGFYPSDIAV
EWESNGQPENNYKTTPPVLDSDGSFFLYSKLTVDKSRWQQGNVFSCSVMH
EALHNHYTQKSLSLSPGKKDPKFWVLVVVGGVLACYSLLVTVAFIIFWVR
SKRSRLLHSDYMNMTPRRPGPTRKHYQPYAPPRDFAAYRSRVKF

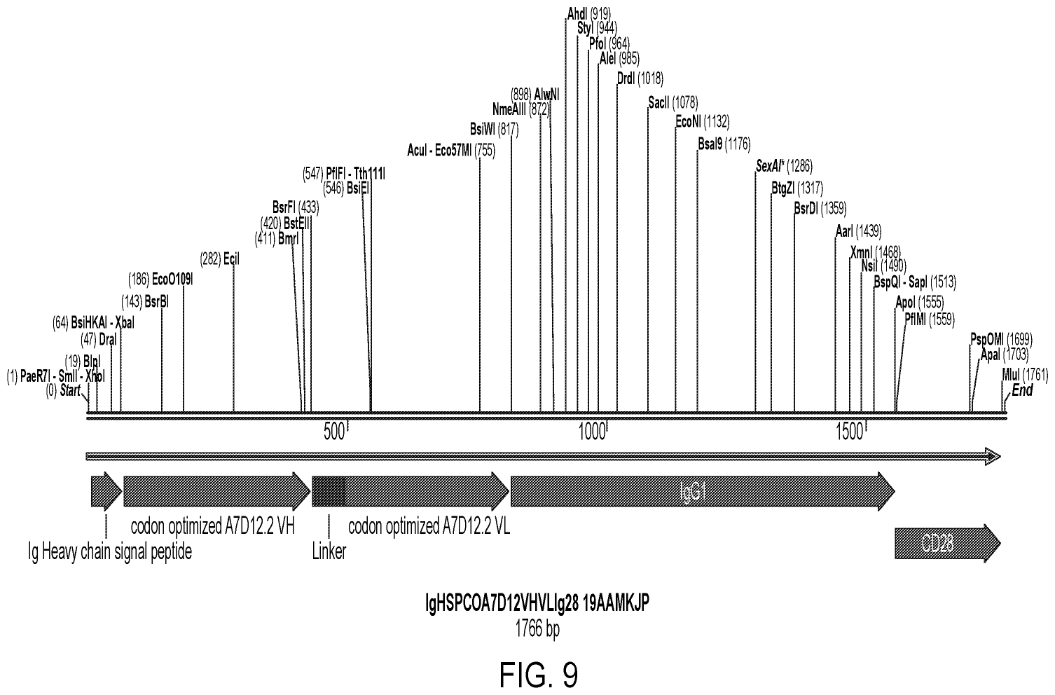

FIG. 9 provides an illustration of an expression vector that encodes a BCMA-targeting CAR with codon-optimized A7D12.2 VH chain linked in a 5′ to 3′ direction to codon-optimized A7D12.2 VL chain and utilizing an Ig heavy chain signal peptide, an IgG1 hinge and CD28 costimulatory domain.

An example of a polynucleotide that encodes an expression construct for IgHSPCOA7D12VHVLIg28 is as follows:

(SEQ ID NO: 60)
CTCGAGATGGAGTTTGGGCTGAGCTGGCTTTTTCTTGTGGCTATTTTAAA
AGGTGTCCAGTGCTCTAGACAGATACAGCTCGTCCAATCCGGTCCCGATT
TGAAAAAGCCTGGCGAAACAGTTAAACTGTCATGTAAGGCGAGCGGATAC
ACGTTTACGAACTTCGGGATGAATTGGGTAAAACAGGCCCCGGGAAAAGG
TTTTAAGTGGATGGCTTGGATAAACACCTACACTGGTGAGTCCTACTTCG
CAGACGATTTCAAAGGGCGGTTCGCGTTTTCAGTAGAGACTTCCGCCACA
ACTGCTTATCTCCAAATAAACAACTTGAAGACCGAGGATACGGCAACCTA
CTTTTGCGCTCGGGGCGAGATTTATTATGGATATGACGGCGGGTTCGCTT
ACTGGGGTCAGGGGACGTTGGTTACCGTGTCTGCCGGTGGTGGTGGTTCT
GGTGGTGGTGGTTCTGGCGGCGGCGGCTCCGGTGGTGGTGGATCCGACGT
GGTGATGACGCAGAGCCACCGATTCATGAGTACCTCTGTAGGCGACCGCG
TCTCAATTACTTGTCGAGCGTCTCAGGACGTAAATACAGCGGTGAGCTGG
TATCAGCAAAAGCCCGGACAGAGCCCGAAATTGCTGATCTTTTCAGCCTC
ATACAGATATACCGGAGTCCCAGACCGCTTTACAGGTTCCGGTAGTGGCG
CGGACTTTACTCTCACAATCAGCTCTGTACAAGCTGAAGATTTGGCTGTT
TACTATTGTCAGCAGCACTATAGTACGCCCTGGACCTTCGGGGGCGGTAC
GAAGTTGGATATTAAGCGTACGGTCACTGTCTCTTCACAGGATCCCGCCG
AGCCCAAATCTCCTGACAAAACTCACACATGCCCACCGTGCCCAGCACCT
GAACTCCTGGGGGGACCGTCAGTCTTCCTCTTCCCCCCAAAACCCAAGGA
CACCCTCATGATCTCCCGGACCCCTGAGGTCACATGCGTGGTGGTGGACG
TGAGCCACGAAGACCCTGAGGTCAAGTTCAACTGGTACGTGGACGGCGTG
GAGGTGCATAATGCCAAGACAAAGCCGCGGGAGGAGCAGTACAACAGCAC
GTACCGTGTGGTCAGCGTCCTCACCGTCCTGCACCAGGACTGGCTGAATG
GCAAGGAGTACAAGTGCAAGGTCTCCAACAAAGCCCTCCCAGCCCCCATC
GAGAAAACCATCTCCAAAGCCAAAGGGCAGCCCCGAGAACCACAGGTGTA
CACCCTGCCCCCATCCCGGGATGAGCTGACCAAGAACCAGGTCAGCCTGA
CCTGCCTGGTCAAAGGCTTCTATCCCAGCGACATCGCCGTGGAGTGGGAG
AGCAATGGGCAACCGGAGAACAACTACAAGACCACGCCTCCCGTGCTGGA
CTCCGACGGCTCCTTCTTCCTCTACAGCAAGCTCACCGTGGACAAGAGCA
GGTGGCAGCAGGGGAACGTCTTCTCATGCTCCGTGATGCATGAGGCTCTG
CACAACCACTACACGCAGAAGAGCCTCTCCCTGTCTCCGGGTAAAAAAGA
TCCCAAATTTTGGGTGCTGGTGGTGGTTGGTGGAGTCCTGGCTTGCTATA
GCTTGCTAGTAACAGTGGCCTTTATTATTTTCTGGGTGAGGAGTAAGAGG
AGCAGGCTCCTGCACAGTGACTACATGAACATGACTCCCCGCCGCCCCGG
GCCCACCCGCAAGCATTACCAGCCCTATGCCCCACCACGCGACTTCGCAG
CCTATCGCTCACGCGT

An example of a polypeptide for IgHSPCOA7D12VHVLIg28 is as follows:

(SEQ ID NO: 61)
LEMEFGLSWLFLVAILKGVQCSRQIQLVQSGPDLKKPGETVKLSCKASGY
TFTNFGMNWVKQAPGKGFKWMAWINTYTGESYFADDFKGRFAFSVETSAT
TAYLQINNLKTEDTATYFCARGEIYYGYDGGFAYWGQGTLVTVSAGGGGS
GGGGSGGGGSGGGGSDVVMTQSHRFMSTSVGDRVSITCRASQDVNTAVSW
YQQKPGQSPKLLIFSASYRYTGVPDRFTGSGSGADFTLTISSVQAEDLAV
YYCQQHYSTPWTFGGGTKLDIKRTVTVSSQDPAEPKSPDKTHTCPPCPAP
ELLGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPI
EKTISKAKGQPREPQVYTLPPSRDELTKNQVSLTCLVKGFYPSDIAVEWE
SNGQPENNYKTTPPVLDSDGSFFLYSKLTVDKSRWQQGNVFSCSVMHEAL
HNHYTQKSLSLSPGKKDPKFWVLVVVGGVLACYSLLVTVAFIIFWVRSKR
SRLLHSDYMNMTPRRPGPTRKHYQPYAPPRDFAAYRSR

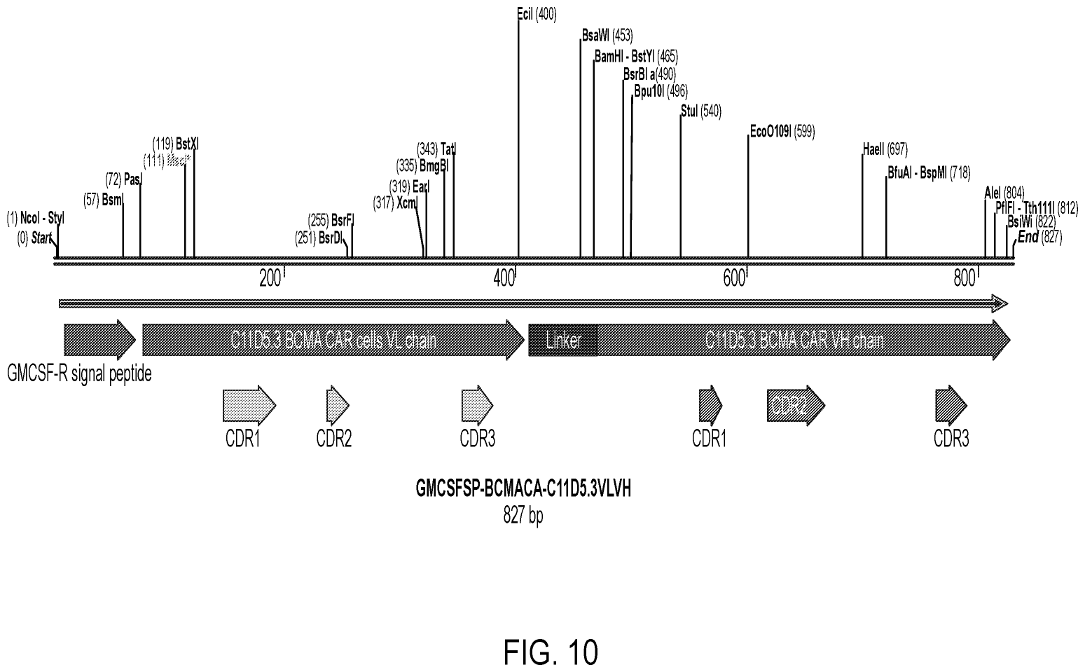

FIG. 10 is an illustration of an expression vector that encodes a BCMA-targeting CAR with C11D5.3 VL chain linked in a 5′ to 3′ direction to C11D5.3 VH chain, with the CAR utilizing GMCSF-R signal peptide.

An example of a polynucleotide that encodes an expression construct for GMCSFSP-BCMAC11D5.3VLVH is as follows:

(SEQ ID NO: 62)
CCATGGGGATGCTTCTCCTGGTGACAAGCCTTCTGCTCTGTGAGTTACCA
CACCCAGCATTCCTCCTGATCCCAGggGACATCGTGCTGACCCAGAGCCC
CCCCAGCCTGGCCATGTCTCTGGGCAAGAGAGCCACCATCAGCTGCCGGG
CCAGCGAGAGCGTGACCATCCTGGGCAGCCACCTGATCCACTGGTATCAG
CAGAAGCCCGGCCAGCCCCCCACCCTGCTGATCCAGCTCGCCAGCAATGT
GCAGACCGGCGTGCCCGCCAGATTCAGCGGCAGCGGCAGCAGAACCGACT
TCACCCTGACCATCGACCCCGTGGAAGAGGACGACGTGGCCGTGTACTAC
TGCCTGCAGAGCCGGACCATCCCCCGGACCTTTGGCGGAGGCACCAAACT
GGAAATCAAGGGTGGTGGTGGTTCTGGTGGTGGTGGTTCTGGCGGCGGCG
GCTCCGGTGGTGGTGGATCCCAGATTCAGCTGGTGCAGAGCGGCCCTGAG
CTGAAGAAACCCGGCGAGACAGTGAAGATCAGCTGCAAGGCCTCCGGCTA
CACCTTCACCGACTACAGCATCAACTGGGTGAAAAGAGCCCCTGGCAAGG
GCCTGAAGTGGATGGGCTGGATCAACACCGAGACAAGAGAGCCCGCCTAC
GCCTACGACTTCCGGGGCAGATTCGCCTTCAGCCTGGAAACCAGCGCCAG
CACCGCCTACCTGCAGATCAACAACCTGAAGTACGAGGACACCGCCACCT
ACTTTTGCGCCCTGGACTACAGCTACGCtATGGACTACTGGGGCCAGGGC
ACCAGCGTGACCGTGTCCAGCCGTACG

An example of a polypeptide for GMCSFSP-BCMAC11D5.3VLVH is as follows:

(SEQ ID NO: 63)
MGMLLLVTSLLLCELPHPAFLLIPGDIVLTQSPPSLAMSLGKRATISCRA
SESVTILGSHLIHWYQQKPGQPPTLLIQLASNVQTGVPARFSGSGSRTDF
TLTIDPVEEDDVAVYYCLQSRTIPRTFGGGTKLEIKGGGGSGGGGSGGGG
SGGGGSQIQLVQSGPELKKPGETVKISCKASGYTFTDYSINWVKRAPGKG
LKWMGWINTETREPAYAYDFRGRFAFSLETSASTAYLQINNLKYEDTATY
FCALDYSYAMDYWGQGTSVTVSSRT

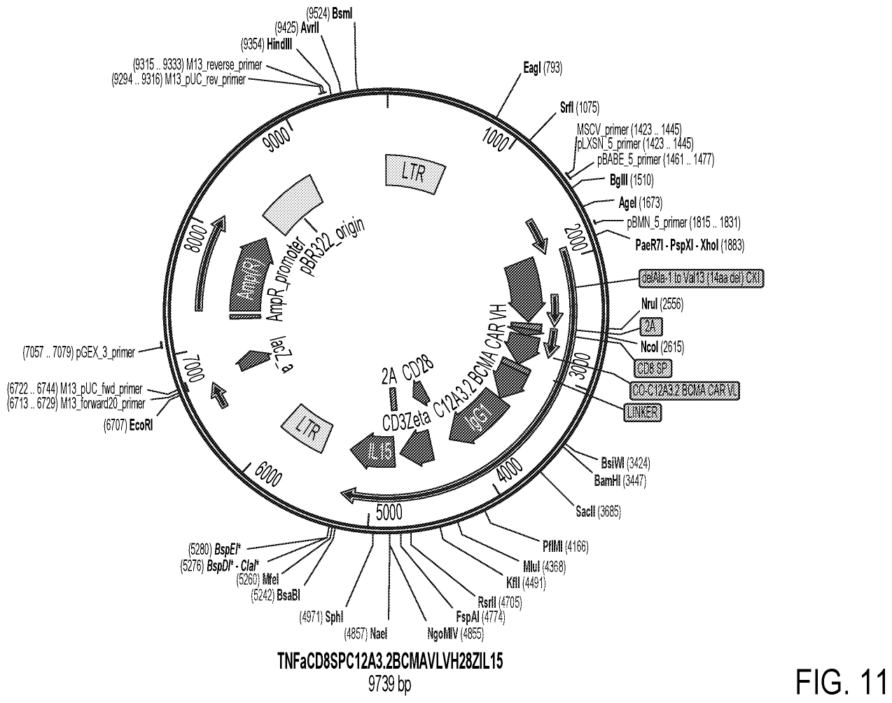

FIG. 11 shows an illustration of a plasmid map of an expression vector encoding a BCMA-targeting CAR utilizing codon-optimized (CO) C12A3.2 VL chain linked in a 5′ to 3′ direction to codon-optimized C12A3.2 VH chain, wherein the CAR incorporates the CD8 signal peptide, the IgG1 hinge, CD28, and CD3zeta. The vector also encodes a particular TNFalpha mutant, delAla-1 to Val13 (14aa del) CKI mutant 5aa mut and encodes IL15. IL15 and the TNFalpha mutant are separated from the CAR by 2A peptides sequences.

An example of a polynucleotide that encodes an expression construct for TNFamut-CD8spC12A3.2.BCMAVLVH is as follows:

(SEQ ID NO: 64)
ATGAGCACTGAAATGCATCCCGGAAGGGGGTCCTGGCACGAGGAGGCGCT
CCCCAAGAAGACAGGGGGGCCCCAGGGCTCCAGGCGGTGCTTGTTCCTCA
GCCTCTTCTCCTTCCTGATCGTGGCAGGCGCCACCACGCTCTTCTTCCTG
CTGCACTTTGGAGTGATCGGCCCCCAGAGGGAAGAGTTCCCCAGGGACCT
CTCTCTAATCAGCCCTCTGCAGGCAGCCCATGTTGTAGCAAACCCTCAAG
CTGAGGGGCAGCTCCAGTGGCTGAACCGCCGGGCCAATGCCCTCCTGGCC
AATGGCGTGGAGCTGAGAGATAACCAGCTGGTTGTGCCATCAGAGGGCCT
GTACCTCATCTACTCCCAGGTCCTCTTCAAGGGCCAAGGCTGCCCCTCCA
CCCATGTGCTCCTCACCCACACCATCAGCCGCATCGCCGTCTCCCACCAG
ACCAAGGTCAACCTCCTCTTCGCCATCAAGAGCCCCTGCCAGAGGGAGAC
CCCAGAGGGGGCTGAGGCTAAGCCCTGGTATGAGCCCATCTATCTGGGAG
GGGTCTTCCAGCTGGAGAAGGGTGACCGACTCATCGCTGAGATCAATCGG
CCCGACTATCTCTACTTTGCCGAGTATGGGCAGGTCTACTTTGGGATCAT
TGCCCTGTCGTCGCGAGCCGAGGGCAGGGGAAGTCTTCTAACATGCGGGG
ACGTGGAGGAAAATCCCGGGCCCATGGGGATGGCCCTGCCTGTGACAGCT
CTGCTGCTGCCCCTGGCCCTGCTGCTCCATGCCGCCAGACCCGACATCGT
GCTGACCCAGAGCCCCCCCAGCCTGGCCATGTCTCTGGGCAAGAGAGCCA
CCATCAGCTGCCGGGCCAGCGAGAGCGTGACCATCCTGGGCAGCCACCTG
ATCTACTGGTATCAGCAGAAGCCTGGCCAGCCCCCCACCCTGCTGATCCA
GCTGGCTAGCAATGTGCAGACCGGCGTGCCCGCCAGATTCAGCGGCAGCG
GCAGCAGAACCGACTTCACCCTGACCATCGACCCCGTGGAAGAGGACGAC
GTGGCCGTGTACTACTGCCTGCAGAGCCGGACCATCCCCCGGACCTTTGG
CGGAGGAACAAAGCTGGAAATCAAGGGCAGCACCAGCGGCTCCGGCAAGC
CTGGCTCTGGCGAGGGCAGCACAAAGGGACAGATTCAGCTGGTGCAGAGC
GGCCCTGAGCTGAAGAAACCCGGCGAGACAGTGAAGATCAGCTGCAAGGC
CTCCGGCTACACCTTCCGGCACTACAGCATGAACTGGGTGAAACAGGCCC
CTGGCAAGGGCCTGAAGTGGATGGGCCGGATCAACACCGAGAGCGGCGTG
CCCATCTACGCCGACGACTTCAAGGGCAGATTCGCCTTCAGCGTGGAAAC
CAGCGCCAGCACCGCCTACCTGGTGATCAACAACCTGAAGGACGAGGATA
CCGCCAGCTACTTCTGCAGCAACGACTACCTGTACAGCCTGGACTTCTGG
GGCCAGGGCACCGCCCTGACCGTGTCCAGCCGTACGGTCACTGTCTCTTC
ACAGGATCCCGCCGAGCCCAAATCTCCTGACAAAACTCACACATGCCCAC
CGTGCCCAGCACCTGAACTCCTGGGGGGACCGTCAGTCTTCCTCTTCCCC
CCAAAACCCAAGGACACCCTCATGATCTCCCGGACCCCTGAGGTCACATG
CGTGGTGGTGGACGTGAGCCACGAAGACCCTGAGGTCAAGTTCAACTGGT
ACGTGGACGGCGTGGAGGTGCATAATGCCAAGACAAAGCCGCGGGAGGAG
CAGTACAACAGCACGTACCGTGTGGTCAGCGTCCTCACCGTCCTGCACCA
GGACTGGCTGAATGGCAAGGAGTACAAGTGCAAGGTCTCCAACAAAGCCC
TCCCAGCCCCCATCGAGAAAACCATCTCCAAAGCCAAAGGGCAGCCCCGA
GAACCACAGGTGTACACCCTGCCCCCATCCCGGGATGAGCTGACCAAGAA
CCAGGTCAGCCTGACCTGCCTGGTCAAAGGCTTCTATCCCAGCGACATCG
CCGTGGAGTGGGAGAGCAATGGGCAACCGGAGAACAACTACAAGACCACG
CCTCCCGTGCTGGACTCCGACGGCTCCTTCTTCCTCTACAGCAAGCTCAC
CGTGGACAAGAGCAGGTGGCAGCAGGGGAACGTCTTCTCATGCTCCGTGA
TGCATGAGGCTCTGCACAACCACTACACGCAGAAGAGCCTCTCCCTGTCT
CCGGGTAAAAAAGATCCCAAATTTTGGGTGCTGGTGGTGGTTGGTGGAGT
CCTGGCTTGCTATAGCTTGCTAGTAACAGTGGCCTTTATTATTTTCTGGG
TGAGGAGTAAGAGGAGCAGGCTCCTGCACAGTGACTACATGAACATGACT
CCCCGCCGCCCCGGGCCCACCCGCAAGCATTACCAGCCCTATGCCCCACC
ACGCGACTTCGCAGCCTATCGCTCACGCGTGAAGTTCAGCAGGAGCGCAG
ACGCCCCCGCGTACCAGCAGGGCCAGAACCAGCTCTATAACGAGCTCAAT
CTAGGACGAAGAGAGGAGTACGATGTTTTGGACAAAAGACGTGGCCGGGA
CCCTGAGATGGGGGGAAAGCCGAGAAGGAAGAACCCTCAGGAAGGCCTGT
ACAATGAACTGCAGAAAGATAAGATGGCGGAGGCCTACAGTGAGATTGGG
ATGAAAGGCGAGCGCCGGAGGGGCAAGGGGCACGATGGCCTTTACCAGGG
TCTCAGTACAGCCACCAAGGACACCTACGACGCCCTTCACATGCAGGCCC
TGCCCCCTCGCGGACCGCAGTGTACTAATTATGCTCTCTTGAAATTGGCT
GGAGATGTTGAGAGCAATCCCGGGCCCATGCGCATTAGCAAGCCCCACCT
GCGGAGCATCAGCATCCAGTGCTACCTGTGCCTGCTGCTGAACAGCCACT
TCCTGACCGAGGCCGGCATCCACGTGTTCATCCTGGGCTGCTTCAGCGCC
GGACTGCCCAAGACCGAGGCCAACTGGGTGAACGTGATCAGCGACCTGAA
GAAGATCGAGGACCTGATCCAGAGCATGCACATCGACGCCACCCTGTACA
CCGAGAGCGACGTGCACCCCAGCTGCAAGGTGACCGCCATGAAGTGCTTT
CTGCTGGAACTGCAGGTGATCAGCCTGGAAAGCGGCGACGCCAGCATCCA
CGACACCGTGGAGAACCTGATCATCCTGGCCAACAACAGCCTGAGCAGCA
ACGGCAACGTGACCGAGAGCGGCTGCAAAGAGTGCGAGGAACTGGAAGAG
AAGAACATCAAAGAGTTTCTGCAGAGCTTCGTGCACATCGTGCAGATGTT
CATCAACACCAGCTGA

An example of a polypeptide for TNFamut-CD8spC12A3.2.BCMAVLVH is as follows:

(SEQ ID NO: 65)
MSTEMHPGRGSWHEEALPKKTGGPQGSRRCLFLSLFSFLIVAGATTLFFL
LHFGVIGPQREEFPRDLSLISPLQAAHVVANPQAEGQLQWLNRRANALLA
NGVELRDNQLVVPSEGLYLIYSQVLFKGQGCPSTHVLLTHTISRIAVSHQ
TKVNLLFAIKSPCQRETPEGAEAKPWYEPIYLGGVFQLEKGDRLIAEINR
PDYLYFAEYGQVYFGIIALSSRAEGRGSLLTCGDVEENPGPMGMALPVTA
LLLPLALLLHAARPDIVLTQSPPSLAMSLGKRATISCRASESVTILGSHL
IYWYQQKPGQPPTLLIQLASNVQTGVPARFSGSGSRTDFTLTIDPVEEDD
VAVYYCLQSRTIPRTFGGGTKLEIKGSTSGSGKPGSGEGSTKGQIQLVQS
GPELKKPGETVKISCKASGYTFRHYSMNWVKQAPGKGLKWMGRINTESGV
PIYADDFKGRFAFSVETSASTAYLVINNLKDEDTASYFCSNDYLYSLDFW
GQGTALTVSSRTVTVSSQDPAEPKSPDKTHTCPPCPAPELLGGPSVFLFP
PKPKDTLMISRTPEVTCVVVDVSHEDPEVKFNWYVDGVEVHNAKTKPREE
QYNSTYRVVSVLTVLHQDWLNGKEYKCKVSNKALPAPIEKTISKAKGQPR
EPQVYTLPPSRDELTKNQVSLTCLVKGFYPSDIAVEWESNGQPENNYKTT
PPVLDSDGSFFLYSKLTVDKSRWQQGNVFSCSVMHEALHNHYTQKSLSLS
PGKKDPKFWVLVVVGGVLACYSLLVTVAFIIFWVRSKRSRLLHSDYMNMT
PRRPGPTRKHYQPYAPPRDFAAYRSRVKFSRSADAPAYQQGQNQLYNELN
LGRREEYDVLDKRRGRDPEMGGKPRRKNPQEGLYNELQKDKMAEAYSEIG
MKGERRRGKGHDGLYQGLSTATKDTYDALHMQALPPRGPQCTNYALLKLA
GDVESNPGPMRISKPHLRSISIQCYLCLLLNSHFLTEAGIHVFILGCFSA
GLPKTEANWVNVISDLKKIEDLIQSMHIDATLYTESDVHPSCKVTAMKCF
LLELQVISLESGDASIHDTVENLIILANNSLSSNGNVTESGCKECEELEE
KNIKEFLQSFVHIVQMFINTS*

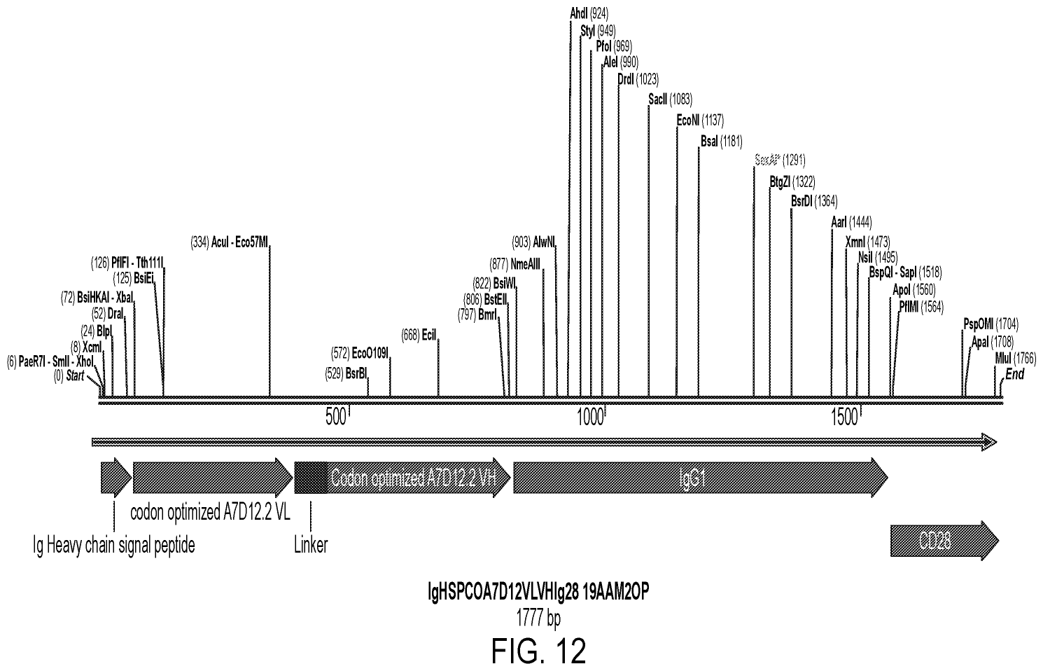

FIG. 12 provides an illustration of an expression vector that encodes a BCMA-targeting CAR with codon optimized A7D12.2 VL linked in a 5′ to 3′ direction to A7D12.2 VH and utilizing the Ig heavy chain signal peptide, IgG1 hinge, and CD28 costimulatory domain.

An example of a polynucleotide that encodes an expression construct for IgHSPCOA7D12VLVHIg28 is as follows:

(SEQ ID NO: 66)
CCATGCTCGAGATGGAGTTTGGGCTGAGCTGGCTTTTTCTTGTGGCTATT
TTAAAAGGTGTCCAGTGCTCTAGAGACGTGGTGATGACGCAGAGCCACCG
ATTCATGAGTACCTCTGTAGGCGACCGCGTCTCAATTACTTGTCGAGCGT
CTCAGGACGTAAATACAGCGGTGAGCTGGTATCAGCAAAAGCCCGGACAG
AGCCCGAAATTGCTGATCTTTTCAGCCTCATACAGATATACCGGAGTCCC
AGACCGCTTTACAGGTTCCGGTAGTGGCGCGGACTTTACTCTCACAATCA
GCTCTGTACAAGCTGAAGATTTGGCTGTTTACTATTGTCAGCAGCACTAT
AGTACGCCCTGGACCTTCGGGGGCGGTACGAAGTTGGATATTAAGGGTGG
TGGTGGTTCTGGTGGTGGTGGTTCTGGCGGCGGCGGCTCCGGTGGTGGTG
GATCCCAGATACAGCTCGTCCAATCCGGTCCCGATTTGAAAAAGCCTGGC
GAAACAGTTAAACTGTCATGTAAGGCGAGCGGATACACGTTTACGAACTT
CGGGATGAATTGGGTAAAACAGGCCCCGGGAAAAGGTTTTAAGTGGATGG
CTTGGATAAACACCTACACTGGTGAGTCCTACTTCGCAGACGATTTCAAA
GGGCGGTTCGCGTTTTCAGTAGAGACTTCCGCCACAACTGCTTATCTCCA
AATAAACAACTTGAAGACCGAGGATACGGCAACCTACTTTTGCGCTCGGG
GCGAGATTTATTATGGATATGACGGCGGGTTCGCTTACTGGGGTCAGGGG
ACGTTGGTTACCGTGTCTGCCCGTACGGTCACTGTCTCTTCACAGGATCC
CGCCGAGCCCAAATCTCCTGACAAAACTCACACATGCCCACCGTGCCCAG
CACCTGAACTCCTGGGGGGACCGTCAGTCTTCCTCTTCCCCCCAAAACCC
AAGGACACCCTCATGATCTCCCGGACCCCTGAGGTCACATGCGTGGTGGT
GGACGTGAGCCACGAAGACCCTGAGGTCAAGTTCAACTGGTACGTGGACG
GCGTGGAGGTGCATAATGCCAAGACAAAGCCGCGGGAGGAGCAGTACAAC
AGCACGTACCGTGTGGTCAGCGTCCTCACCGTCCTGCACCAGGACTGGCT
GAATGGCAAGGAGTACAAGTGCAAGGTCTCCAACAAAGCCCTCCCAGCCC
CCATCGAGAAAACCATCTCCAAAGCCAAAGGGCAGCCCCGAGAACCACAG
GTGTACACCCTGCCCCCATCCCGGGATGAGCTGACCAAGAACCAGGTCAG
CCTGACCTGCCTGGTCAAAGGCTTCTATCCCAGCGACATCGCCGTGGAGT
GGGAGAGCAATGGGCAACCGGAGAACAACTACAAGACCACGCCTCCCGTG
CTGGACTCCGACGGCTCCTTCTTCCTCTACAGCAAGCTCACCGTGGACAA
GAGCAGGTGGCAGCAGGGGAACGTCTTCTCATGCTCCGTGATGCATGAGG
CTCTGCACAACCACTACACGCAGAAGAGCCTCTCCCTGTCTCCGGGTAAA
AAAGATCCCAAATTTTGGGTGCTGGTGGTGGTTGGTGGAGTCCTGGCTTG
CTATAGCTTGCTAGTAACAGTGGCCTTTATTATTTTCTGGGTGAGGAGTA
AGAGGAGCAGGCTCCTGCACAGTGACTACATGAACATGACTCCCCGCCGC
CCCGGGCCCACCCGCAAGCATTACCAGCCCTATGCCCCACCACGCGACTT
CGCAGCCTATCGCTCACGCGTGAAGTT

An example of a polypeptide for IgHSPCOA7D12VLVHIg28 is as follows:

(SEQ ID NO: 67)
MLEMEFGLSWLFLVAILKGVQCSRDVVMTQSHRFMSTSVGDRVSITCRAS
QDVNTAVSWYQQKPGQSPKLLIFSASYRYTGVPDRFTGSGSGADFTLTIS
SVQAEDLAVYYCQQHYSTPWTFGGGTKLDIKGGGGSGGGGSGGGGSGGGG
SQIQLVQSGPDLKKPGETVKLSCKASGYTFTNFGMNWVKQAPGKGFKWMA
WINTYTGESYFADDFKGRFAFSVETSATTAYLQINNLKTEDTATYFCARG
EIYYGYDGGFAYWGQGTLVTVSARTVTVSSQDPAEPKSPDKTHTCPPCPA
PELLGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALPAP
IEKTISKAKGQPREPQVYTLPPSRDELTKNQVSLTCLVKGFYPSDIAVEW
ESNGQPENNYKTTPPVLDSDGSFFLYSKLTVDKSRWQQGNVFSCSVMHEA
LHNHYTQKSLSLSPGKKDPKFWVLVVVGGVLACYSLLVTVAFIIFWVRSK
RSRLLHSDYMNMTPRRPGPTRKHYQPYAPPRDFAAYRSRVK

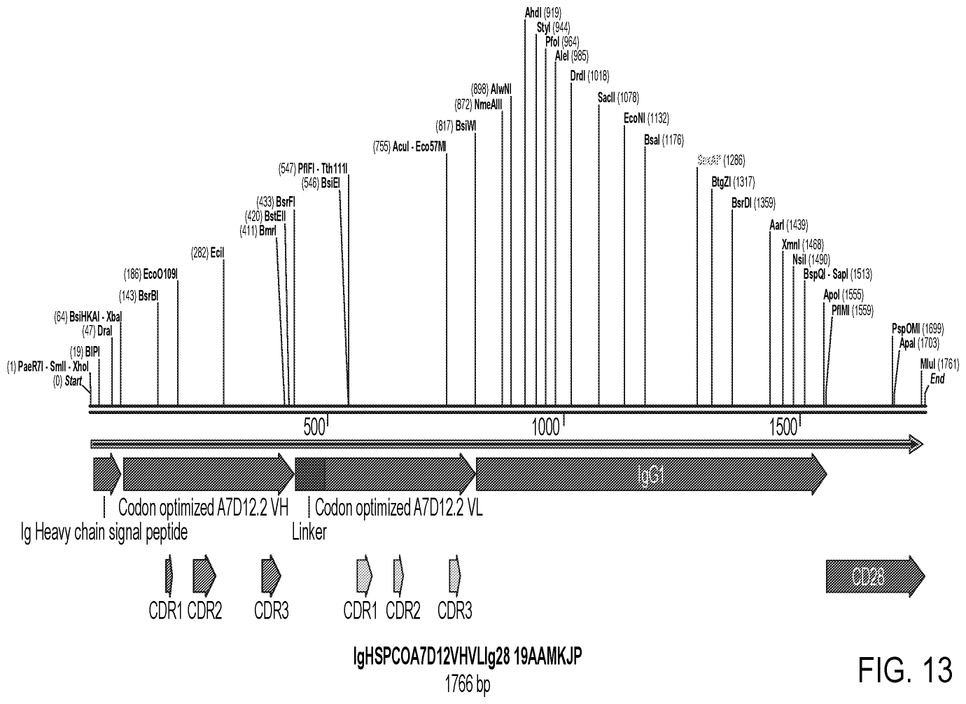

FIG. 13 provides an illustration of an expression vector that encodes a BCMA-targeting CAR with codon optimized A7D12.2 VH linked in a 5′ to 3′ direction to codon optimized A7D12.2 VL in which case the CAR also employs the Ig Heavy Chain signal peptide, the IgG1 hinge, and CD28 costimulatory domain.

An example of a IgHSPCoA7D12VHVLIg28 nucleic acid sequence is as follows:

(SEQ ID NO: 151)
CTCGAGATGGAGTTTGGGCTGAGCTGGCTTTTTCTTGTGGCTATTTTAAA
AGGTGTCCAGTGCTCTAGACAGATACAGCTCGTCCAATCCGGTCCCGATT
TGAAAAAGCCTGGCGAAACAGTTAAACTGTCATGTAAGGCGAGCGGATAC
ACGTTTACGAACTTCGGGATGAATTGGGTAAAACAGGCCCCGGGAAAAGG
TTTTAAGTGGATGGCTTGGATAAACACCTACACTGGTGAGTCCTACTTCG
CAGACGATTTCAAAGGGCGGTTCGCGTTTTCAGTAGAGACTTCCGCCACA
ACTGCTTATCTCCAAATAAACAACTTGAAGACCGAGGATACGGCAACCTA
CTTTTGCGCTCGGGGCGAGATTTATTATGGATATGACGGCGGGTTCGCTT
ACTGGGGTCAGGGGACGTTGGTTACCGTGTCTGCCGGTGGTGGTGGTTCT
GGTGGTGGTGGTTCTGGCGGCGGCGGCTCCGGTGGTGGTGGATCCGACGT
GGTGATGACGCAGAGCCACCGATTCATGAGTACCTCTGTAGGCGACCGCG
TCTCAATTACTTGTCGAGCGTCTCAGGACGTAAATACAGCGGTGAGCTGG
TATCAGCAAAAGCCCGGACAGAGCCCGAAATTGCTGATCTTTTCAGCCTC
ATACAGATATACCGGAGTCCCAGACCGCTTTACAGGTTCCGGTAGTGGCG
CGGACTTTACTCTCACAATCAGCTCTGTACAAGCTGAAGATTTGGCTGTT
TACTATTGTCAGCAGCACTATAGTACGCCCTGGACCTTCGGGGGCGGTAC
GAAGTTGGATATTAAGCGTACGGTCACTGTCTCTTCACAGGATCCCGCCG
AGCCCAAATCTCCTGACAAAACTCACACATGCCCACCGTGCCCAGCACCT
GAACTCCTGGGGGGACCGTCAGTCTTCCTCTTCCCCCCAAAACCCAAGGA
CACCCTCATGATCTCCCGGACCCCTGAGGTCACATGCGTGGTGGTGGACG
TGAGCCACGAAGACCCTGAGGTCAAGTTCAACTGGTACGTGGACGGCGTG
GAGGTGCATAATGCCAAGACAAAGCCGCGGGAGGAGCAGTACAACAGCAC
GTACCGTGTGGTCAGCGTCCTCACCGTCCTGCACCAGGACTGGCTGAATG
GCAAGGAGTACAAGTGCAAGGTCTCCAACAAAGCCCTCCCAGCCCCCATC
GAGAAAACCATCTCCAAAGCCAAAGGGCAGCCCCGAGAACCACAGGTGTA
CACCCTGCCCCCATCCCGGGATGAGCTGACCAAGAACCAGGTCAGCCTGA
CCTGCCTGGTCAAAGGCTTCTATCCCAGCGACATCGCCGTGGAGTGGGAG
AGCAATGGGCAACCGGAGAACAACTACAAGACCACGCCTCCCGTGCTGGA
CTCCGACGGCTCCTTCTTCCTCTACAGCAAGCTCACCGTGGACAAGAGCA
GGTGGCAGCAGGGGAACGTCTTCTCATGCTCCGTGATGCATGAGGCTCTG
CACAACCACTACACGCAGAAGAGCCTCTCCCTGTCTCCGGGTAAAAAAGA
TCCCAAATTTTGGGTGCTGGTGGTGGTTGGTGGAGTCCTGGCTTGCTATA
GCTTGCTAGTAACAGTGGCCTTTATTATTTTCTGGGTGAGGAGTAAGAGG
AGCAGGCTCCTGCACAGTGACTACATGAACATGACTCCCCGCCGCCCCGG
GCCCACCCGCAAGCATTACCAGCCCTATGCCCCACCACGCGACTTCGCAG
CCTATCGCTCACGCGT

An example of a IgHSPCoA7D12VHVLIg28 polypeptide sequence is as follows:

(SEQ ID NO: 152)
LEMEFGLSWLFLVAILKGVQCSRQIQLVQSGPDLKKPGETVKLSCKASGY
TFTNFGMNWVKQAPGKGFKWMAWINTYTGESYFADDFKGRFAFSVETSAT
TAYLQINNLKTEDTATYFCARGEIYYGYDGGFAYWGQGTLVTVSAGGGGS
GGGGSGGGGSGGGGSDVVMTQSHRFMSTSVGDRVSITCRASQDVNTAVSW
YQQKPGQSPKLLIFSASYRYTGVPDRFTGSGSGADFTLTISSVQAEDLAV
YYCQQHYSTPWTFGGGTKLDIKRTVTVSSQDPAEPKSPDKTHTCPPCPAP
ELLGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPI
EKTISKAKGQPREPQVYTLPPSRDELTKNQVSLTCLVKGFYPSDIAVEWE
SNGQPENNYKTTPPVLDSDGSFFLYSKLTVDKSRWQQGNVFSCSVMHEAL
HNHYTQKSLSLSPGKKDPKFWVLVVVGGVLACYSLLVTVAFIIFWVRSKR
SRLLHSDYMNMTPRRPGPTRKHYQPYAPPRDFAAYRSR

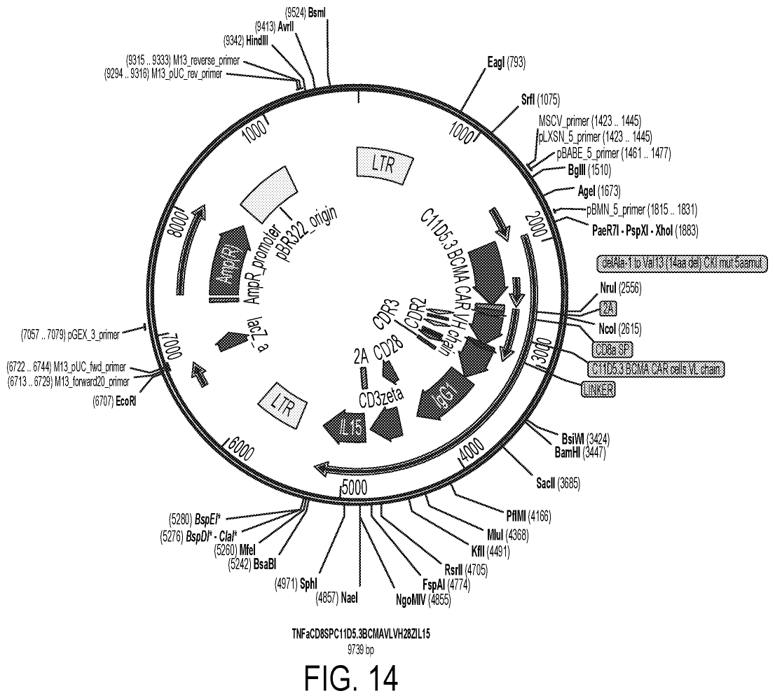

An example of an expression vector that encodes a BCMA-targeting CAR that utilizes the CD8 signal peptide, the C11D5.3 VL chain linked in a 5′ to 3′ direction with the VH heavy chain, wherein the CAR also uses the IgG1 hinge. FIG. 14 illustrates a version of the expression construct in which a TNFalpha mutant and IL15 are separated from the CAR sequences to produce separate polypeptides.

An example of a CD8spC11D5.3VLVHIgG128zIL15 expression construct polynucleotide is as follows:

(SEQ ID NO: 153)
ATGAGCACTGAAATGCATCCCGGAAGGGGGTCCTGGCACGAGGAGGCGCT
CCCCAAGAAGACAGGGGGGCCCCAGGGCTCCAGGCGGTGCTTGTTCCTCA
GCCTCTTCTCCTTCCTGATCGTGGCAGGCGCCACCACGCTCTTCTTCCTG
CTGCACTTTGGAGTGATCGGCCCCCAGAGGGAAGAGTTCCCCAGGGACCT
CTCTCTAATCAGCCCTCTGCAGGCAGCCCATGTTGTAGCAAACCCTCAAG
CTGAGGGGCAGCTCCAGTGGCTGAACCGCCGGGCCAATGCCCTCCTGGCC
AATGGCGTGGAGCTGAGAGATAACCAGCTGGTTGTGCCATCAGAGGGCCT
GTACCTCATCTACTCCCAGGTCCTCTTCAAGGGCCAAGGCTGCCCCTCCA
CCCATGTGCTCCTCACCCACACCATCAGCCGCATCGCCGTCTCCCACCAG
ACCAAGGTCAACCTCCTCTTCGCCATCAAGAGCCCCTGCCAGAGGGAGAC
CCCAGAGGGGGCTGAGGCTAAGCCCTGGTATGAGCCCATCTATCTGGGAG
GGGTCTTCCAGCTGGAGAAGGGTGACCGACTCATCGCTGAGATCAATCGG
CCCGACTATCTCTACTTTGCCGAGTATGGGCAGGTCTACTTTGGGATCAT
TGCCCTGTCGTCGCGAGCCGAGGGCAGGGGAAGTCTTCTAACATGCGGGG
ACGTGGAGGAAAATCCCGGGCCCATGGGGATGGCCCTGCCTGTGACAGCT
CTGCTCCTCCCTCTGGCCCTGCTGCTCCATGCCGCCAGACCCGACATCGT
GCTGACCCAGAGCCCCCCCAGCCTGGCCATGTCTCTGGGCAAGAGAGCCA
CCATCAGCTGCCGGGCCAGCGAGAGCGTGACCATCCTGGGCAGCCACCTG
ATCCACTGGTATCAGCAGAAGCCCGGCCAGCCCCCCACCCTGCTGATCCA
GCTCGCCAGCAATGTGCAGACCGGCGTGCCCGCCAGATTCAGCGGCAGCG
GCAGCAGAACCGACTTCACCCTGACCATCGACCCCGTGGAAGAGGACGAC
GTGGCCGTGTACTACTGCCTGCAGAGCCGGACCATCCCCCGGACCTTTGG
CGGAGGCACCAAACTGGAAATCAAGGGCAGCACCAGCGGCTCCGGCAAGC
CTGGCTCTGGCGAGGGCAGCACAAAGGGACAGATTCAGCTGGTGCAGAGC
GGCCCTGAGCTGAAGAAACCCGGCGAGACAGTGAAGATCAGCTGCAAGGC
CTCCGGCTACACCTTCACCGACTACAGCATCAACTGGGTGAAAAGAGCCC
CTGGCAAGGGCCTGAAGTGGATGGGCTGGATCAACACCGAGACAAGAGAG
CCCGCCTACGCCTACGACTTCCGGGGCAGATTCGCCTTCAGCCTGGAAAC
CAGCGCCAGCACCGCCTACCTGCAGATCAACAACCTGAAGTACGAGGACA
CCGCCACCTACTTTTGCGCCCTGGACTACAGCTACGCtATGGACTACTGG
GGCCAGGGCACCAGCGTGACCGTGTCCAGCCGTACGGTCACTGTCTCTTC
ACAGGATCCCGCCGAGCCCAAATCTCCTGACAAAACTCACACATGCCCAC
CGTGCCCAGCACCTGAACTCCTGGGGGGACCGTCAGTCTTCCTCTTCCCC
CCAAAACCCAAGGACACCCTCATGATCTCCCGGACCCCTGAGGTCACATG
CGTGGTGGTGGACGTGAGCCACGAAGACCCTGAGGTCAAGTTCAACTGGT
ACGTGGACGGCGTGGAGGTGCATAATGCCAAGACAAAGCCGCGGGAGGAG
CAGTACAACAGCACGTACCGTGTGGTCAGCGTCCTCACCGTCCTGCACCA
GGACTGGCTGAATGGCAAGGAGTACAAGTGCAAGGTCTCCAACAAAGCCC
TCCCAGCCCCCATCGAGAAAACCATCTCCAAAGCCAAAGGGCAGCCCCGA
GAACCACAGGTGTACACCCTGCCCCCATCCCGGGATGAGCTGACCAAGAA
CCAGGTCAGCCTGACCTGCCTGGTCAAAGGCTTCTATCCCAGCGACATCG
CCGTGGAGTGGGAGAGCAATGGGCAACCGGAGAACAACTACAAGACCACG
CCTCCCGTGCTGGACTCCGACGGCTCCTTCTTCCTCTACAGCAAGCTCAC
CGTGGACAAGAGCAGGTGGCAGCAGGGGAACGTCTTCTCATGCTCCGTGA
TGCATGAGGCTCTGCACAACCACTACACGCAGAAGAGCCTCTCCCTGTCT
CCGGGTAAAAAAGATCCCAAATTTTGGGTGCTGGTGGTGGTTGGTGGAGT
CCTGGCTTGCTATAGCTTGCTAGTAACAGTGGCCTTTATTATTTTCTGGG
TGAGGAGTAAGAGGAGCAGGCTCCTGCACAGTGACTACATGAACATGACT
CCCCGCCGCCCCGGGCCCACCCGCAAGCATTACCAGCCCTATGCCCCACC
ACGCGACTTCGCAGCCTATCGCTCACGCGTGAAGTTCAGCAGGAGCGCAG
ACGCCCCCGCGTACCAGCAGGGCCAGAACCAGCTCTATAACGAGCTCAAT
CTAGGACGAAGAGAGGAGTACGATGTTTTGGACAAAAGACGTGGCCGGGA
CCCTGAGATGGGGGGAAAGCCGAGAAGGAAGAACCCTCAGGAAGGCCTGT
ACAATGAACTGCAGAAAGATAAGATGGCGGAGGCCTACAGTGAGATTGGG
ATGAAAGGCGAGCGCCGGAGGGGCAAGGGGCACGATGGCCTTTACCAGGG
TCTCAGTACAGCCACCAAGGACACCTACGACGCCCTTCACATGCAGGCCC
TGCCCCCTCGCGGACCGCAGTGTACTAATTATGCTCTCTTGAAATTGGCT
GGAGATGTTGAGAGCAATCCCGGGCCCATGCGCATTAGCAAGCCCCACCT
GCGGAGCATCAGCATCCAGTGCTACCTGTGCCTGCTGCTGAACAGCCACT
TCCTGACCGAGGCCGGCATCCACGTGTTCATCCTGGGCTGCTTCAGCGCC
GGACTGCCCAAGACCGAGGCCAACTGGGTGAACGTGATCAGCGACCTGAA
GAAGATCGAGGACCTGATCCAGAGCATGCACATCGACGCCACCCTGTACA
CCGAGAGCGACGTGCACCCCAGCTGCAAGGTGACCGCCATGAAGTGCTTT
CTGCTGGAACTGCAGGTGATCAGCCTGGAAAGCGGCGACGCCAGCATCCA
CGACACCGTGGAGAACCTGATCATCCTGGCCAACAACAGCCTGAGCAGCA
ACGGCAACGTGACCGAGAGCGGCTGCAAAGAGTGCGAGGAACTGGAAGAG
AAGAACATCAAAGAGTTTCTGCAGAGCTTCGTGCACATCGTGCAGATGTT
CATCAACACCAGCTGA

An example of a CD8spC11D5.3VLVHIgG1 expression construct polypeptide is as follows:

(SEQ ID NO: 154)
MSTEMHPGRGSWHEEALPKKTGGPQGSRRCLFLSLFSFLIVAGATTLFFL
LHFGVIGPQREEFPRDLSLISPLQAAHVVANPQAEGQLQWLNRRANALLA
NGVELRDNQLVVPSEGLYLIYSQVLFKGQGCPSTHVLLTHTISRIAVSHQ
TKVNLLFAIKSPCQRETPEGAEAKPWYEPIYLGGVFQLEKGDRLIAEINR
PDYLYFAEYGQVYFGIIALSSRAEGRGSLLTCGDVEENPGPMGMALPVTA
LLLPLALLLHAARPDIVLTQSPPSLAMSLGKRATISCRASESVTILGSHL
IHWYQQKPGQPPTLLIQLASNVQTGVPARFSGSGSRTDFTLTIDPVEEDD
VAVYYCLQSRTIPRTFGGGTKLEIKGSTSGSGKPGSGEGSTKGQIQLVQS
GPELKKPGETVKISCKASGYTFTDYSINWVKRAPGKGLKWMGWINTETRE
PAYAYDFRGRFAFSLETSASTAYLQINNLKYEDTATYFCALDYSYAMDYW
GQGTSVTVSSRTVTVSSQDPAEPKSPDKTHTCPPCPAPELLGGPSVFLFP
PKPKDTLMISRTPEVTCVVVDVSHEDPEVKFNWYVDGVEVHNAKTKPREE
QYNSTYRVVSVLTVLHQDWLNGKEYKCKVSNKALPAPIEKTISKAKGQPR
EPQVYTLPPSRDELTKNQVSLTCLVKGFYPSDIAVEWESNGQPENNYKTT
PPVLDSDGSFFLYSKLTVDKSRWQQGNVFSCSVMHEALHNHYTQKSLSLS
PGKKDPKFWVLVVVGGVLACYSLLVTVAFIIFWVRSKRSRLLHSDYMNMT
PRRPGPTRKHYQPYAPPRDFAAYRSRVKFSRSADAPAYQQGQNQLYNELN
LGRREEYDVLDKRRGRDPEMGGKPRRKNPQEGLYNELQKDKMAEAYSEIG
MKGERRRGKGHDGLYQGLSTATKDTYDALHMQALPPRGPQCTNYALLKLA
GDVESNPGPMRISKPHLRSISIQCYLCLLLNSHFLTEAGIHVFILGCFSA
GLPKTEANWVNVISDLKKIEDLIQSMHIDATLYTESDVHPSCKVTAMKCF
LLELQVISLESGDASIHDTVENLIILANNSLSSNGNVTESGCKECEELEE
KNIKEFLQSFVHIVQMFINTS*

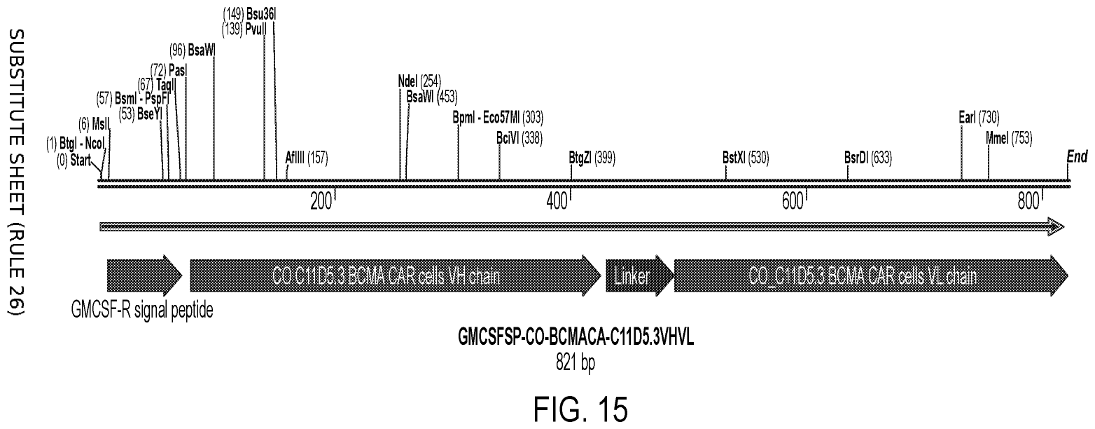

FIG. 15 provides an example of an expression vector that encodes a BCMA-targeting CAR having a GMCSF-R signal peptide and C11D5.3 VH chain linked in a 5′ to 3′ direction to the C11D5.3 VL chain.

An example of GMCSFSPcoC11D5.3VHVLIgG28 polynucleotide is as follows:

(SEQ ID NO: 155)
CCATGGGGATGCTTCTCCTGGTGACAAGCCTTCTGCTCTGTGAGTTACCA
CACCCAGCATTCCTCCTGATCCCAGggCAAATCCAGCTCGTCCAATCCGG
TCCAGAGTTGAAGAAACCCGGCGAGACGGTAAAAATCAGCTGTAAAGCCT
CAGGTTACACGTTTACGGACTATAGCATCAATTGGGTTAAGAGGGCTCCG
GGGAAGGGGCTCAAATGGATGGGCTGGATAAACACAGAGACGAGAGAGCC
CGCATATGCGTACGACTTTAGAGGTCGATTCGCTTTCAGTCTTGAAACCT
CTGCTTCTACCGCGTATCTCCAGATAAACAACCTGAAATATGAGGATACA
GCAACTTATTTTTGCGCTCTCGATTACAGCTATGCGATGGATTATTGGGG
ACAAGGAACTTCCGTGACTGTGTCAAGCGGGGGTGGAGGTTCTGGCGGAG
GGGGCAGCGGTGGTGGAGGAAGTGGGGGCGGTGGGAGTGACATTGTTTTG
ACCCAATCACCTCCCTCTCTCGCCATGTCCTTGGGTAAACGGGCAACAAT
CTCCTGTAGAGCTTCCGAAAGTGTAACAATTCTTGGAAGCCACCTCATAC
ATTGGTATCAGCAAAAGCCGGGGCAGCCCCCTACATTGCTCATTCAATTG
GCTTCAAATGTCCAGACGGGTGTACCAGCGAGATTCTCAGGGAGTGGCTC
CCGAACGGATTTCACACTGACGATTGATCCCGTCGAAGAGGACGATGTCG
CAGTTTATTATTGCCTCCAAAGTCGGACAATTCCGAGGACTTTTGGAGGC
GGAACAAAATTGGAAATCAAA

An example of GMCSFSPcoC11D5.3VHVLIgG28 polypeptide is as follows:

(SEQ ID NO: 156)
MGMLLLVTSLLLCELPHPAFLLIPGQIQLVQSGPE
LKKPGETVKISCKASGYTFTDYSINWVKRAPGKGL
KWMGWINTETREPAYAYDFRGRFAFSLETSASTAY
LQINNLKYEDTATYFCALDYSYAMDYWGQGTSVTV
SSGGGGSGGGGSGGGGSGGGGSDIVLTQSPPSLAM
SLGKRATISCRASESVTILGSHLIHWYQQKPGQPP
TLLIQLASNVQTGVPARFSGSGSRTDFTLTIDPVE
EDDVAVYYCLQSRTIPRTFGGGTKLEIK

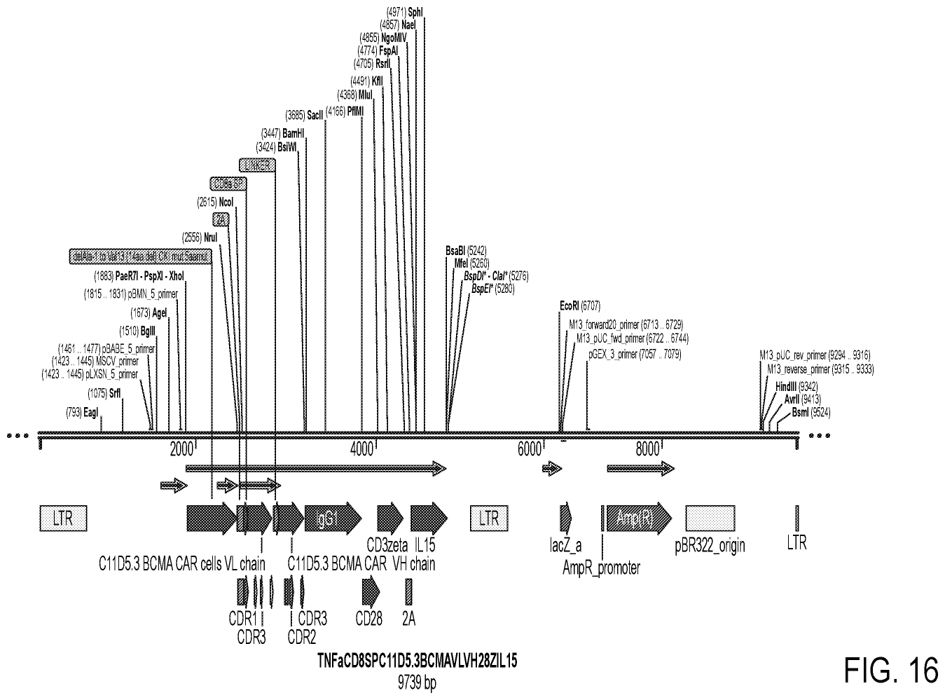

FIG. 16 provides an illustration of an expression vector that encodes a BCMA-targeting CAR, wherein the CAR includes the CD8 signal peptide, the VL and VH chains of C11D5.3 scFv, and CD28 costimulatory domain. The construct also encodes a TNFalpha mutant and IL15 separated from the CAR by 2A sequences.

An example of a TNFaCD8spC11D5.3BCMAVLVH28ZIL15 polynucleotide is as follows:

(SEQ ID NO: 157)
ATGAGCACTGAAATGCATCCCGGAAGGGGGTCCTG
GCACGAGGAGGCGCTCCCCAAGAAGACAGGGGGGC
CCCAGGGCTCCAGGCGGTGCTTGTTCCTCAGCCTC
TTCTCCTTCCTGATCGTGGCAGGCGCCACCACGCT
CTTCTTCCTGCTGCACTTTGGAGTGATCGGCCCCC
AGAGGGAAGAGTTCCCCAGGGACCTCTCTCTAATC
AGCCCTCTGCAGGCAGCCCATGTTGTAGCAAACCC
TCAAGCTGAGGGGCAGCTCCAGTGGCTGAACCGCC
GGGCCAATGCCCTCCTGGCCAATGGCGTGGAGCTG
AGAGATAACCAGCTGGTTGTGCCATCAGAGGGCCT
GTACCTCATCTACTCCCAGGTCCTCTTCAAGGGCC
AAGGCTGCCCCTCCACCCATGTGCTCCTCACCCAC
ACCATCAGCCGCATCGCCGTCTCCCACCAGACCAA
GGTCAACCTCCTCTTCGCCATCAAGAGCCCCTGCC
AGAGGGAGACCCCAGAGGGGGCTGAGGCTAAGCCC
TGGTATGAGCCCATCTATCTGGGAGGGGTCTTCCA
GCTGGAGAAGGGTGACCGACTCATCGCTGAGATCA
ATCGGCCCGACTATCTCTACTTTGCCGAGTATGGG
CAGGTCTACTTTGGGATCATTGCCCTGTCGTCGCG
AGCCGAGGGCAGGGGAAGTCTTCTAACATGCGGGG
ACGTGGAGGAAAATCCCGGGCCCATGGGGATGGCC
CTGCCTGTGACAGCTCTGCTCCTCCCTCTGGCCCT
GCTGCTCCATGCCGCCAGACCCGACATCGTGCTGA
CCCAGAGCCCCCCCAGCCTGGCCATGTCTCTGGGC
AAGAGAGCCACCATCAGCTGCCGGGCCAGCGAGAG
CGTGACCATCCTGGGCAGCCACCTGATCCACTGGT
ATCAGCAGAAGCCCGGCCAGCCCCCCACCCTGCTG
ATCCAGCTCGCCAGCAATGTGCAGACCGGCGTGCC
CGCCAGATTCAGCGGCAGCGGCAGCAGAACCGACT
TCACCCTGACCATCGACCCCGTGGAAGAGGACGAC
GTGGCCGTGTACTACTGCCTGCAGAGCCGGACCAT
CCCCCGGACCTTTGGCGGAGGCACCAAACTGGAAA
TCAAGGGCAGCACCAGCGGCTCCGGCAAGCCTGGC
TCTGGCGAGGGCAGCACAAAGGGACAGATTCAGCT
GGTGCAGAGCGGCCCTGAGCTGAAGAAACCCGGCG
AGACAGTGAAGATCAGCTGCAAGGCCTCCGGCTAC
ACCTTCACCGACTACAGCATCAACTGGGTGAAAAG
AGCCCCTGGCAAGGGCCTGAAGTGGATGGGCTGGA
TCAACACCGAGACAAGAGAGCCCGCCTACGCCTAC
GACTTCCGGGGCAGATTCGCCTTCAGCCTGGAAAC
CAGCGCCAGCACCGCCTACCTGCAGATCAACAACC
TGAAGTACGAGGACACCGCCACCTACTTTTGCGCC
CTGGACTACAGCTACGCtATGGACTACTGGGGCCA
GGGCACCAGCGTGACCGTGTCCAGCCGTACGGTCA
CTGTCTCTTCACAGGATCCCGCCGAGCCCAAATCT
CCTGACAAAACTCACACATGCCCACCGTGCCCAGC
ACCTGAACTCCTGGGGGGACCGTCAGTCTTCCTCT
TCCCCCCAAAACCCAAGGACACCCTCATGATCTCC
CGGACCCCTGAGGTCACATGCGTGGTGGTGGACGT
GAGCCACGAAGACCCTGAGGTCAAGTTCAACTGGT
ACGTGGACGGCGTGGAGGTGCATAATGCCAAGACA
AAGCCGCGGGAGGAGCAGTACAACAGCACGTACCG
TGTGGTCAGCGTCCTCACCGTCCTGCACCAGGACT
GGCTGAATGGCAAGGAGTACAAGTGCAAGGTCTCC
AACAAAGCCCTCCCAGCCCCCATCGAGAAAACCAT
CTCCAAAGCCAAAGGGCAGCCCCGAGAACCACAGG
TGTACACCCTGCCCCCATCCCGGGATGAGCTGACC
AAGAACCAGGTCAGCCTGACCTGCCTGGTCAAAGG
CTTCTATCCCAGCGACATCGCCGTGGAGTGGGAGA
GCAATGGGCAACCGGAGAACAACTACAAGACCACG
CCTCCCGTGCTGGACTCCGACGGCTCCTTCTTCCT
CTACAGCAAGCTCACCGTGGACAAGAGCAGGTGGC
AGCAGGGGAACGTCTTCTCATGCTCCGTGATGCAT
GAGGCTCTGCACAACCACTACACGCAGAAGAGCCT
CTCCCTGTCTCCGGGTAAAAAAGATCCCAAATTTT
GGGTGCTGGTGGTGGTTGGTGGAGTCCTGGCTTGC
TATAGCTTGCTAGTAACAGTGGCCTTTATTATTTT
CTGGGTGAGGAGTAAGAGGAGCAGGCTCCTGCACA
GTGACTACATGAACATGACTCCCCGCCGCCCCGGG
CCCACCCGCAAGCATTACCAGCCCTATGCCCCACC
ACGCGACTTCGCAGCCTATCGCTCACGCGTGAAGT
TCAGCAGGAGCGCAGACGCCCCCGCGTACCAGCAG
GGCCAGAACCAGCTCTATAACGAGCTCAATCTAGG
ACGAAGAGAGGAGTACGATGTTTTGGACAAAAGAC
GTGGCCGGGACCCTGAGATGGGGGGAAAGCCGAGA
AGGAAGAACCCTCAGGAAGGCCTGTACAATGAACT
GCAGAAAGATAAGATGGCGGAGGCCTACAGTGAGA
TTGGGATGAAAGGCGAGCGCCGGAGGGGCAAGGGG
CACGATGGCCTTTACCAGGGTCTCAGTACAGCCAC
CAAGGACACCTACGACGCCCTTCACATGCAGGCCC
TGCCCCCTCGCGGACCGCAGTGTACTAATTATGCT
CTCTTGAAATTGGCTGGAGATGTTGAGAGCAATCC
CGGGCCCATGCGCATTAGCAAGCCCCACCTGCGGA
GCATCAGCATCCAGTGCTACCTGTGCCTGCTGCTG
AACAGCCACTTCCTGACCGAGGCCGGCATCCACGT
GTTCATCCTGGGCTGCTTCAGCGCCGGACTGCCCA
AGACCGAGGCCAACTGGGTGAACGTGATCAGCGAC
CTGAAGAAGATCGAGGACCTGATCCAGAGCATGCA
CATCGACGCCACCCTGTACACCGAGAGCGACGTGC
ACCCCAGCTGCAAGGTGACCGCCATGAAGTGCTTT
CTGCTGGAACTGCAGGTGATCAGCCTGGAAAGCGG
CGACGCCAGCATCCACGACACCGTGGAGAACCTGA
TCATCCTGGCCAACAACAGCCTGAGCAGCAACGGC
AACGTGACCGAGAGCGGCTGCAAAGAGTGCGAGGA
ACTGGAAGAGAAGAACATCAAAGAGTTTCTGCAGA
GCTTCGTGCACATCGTGCAGATGTTCATCAACACC
AGCTGA

An example of a TNFaCD8spC11D5.3BCMAVLVH28ZIL15 polypeptide is as follows:

(SEQ ID NO: 158)
MSTEMHPGRGSWHEEALPKKTGGPQGSRRCLFLSL
FSFLIVAGATTLFFLLHFGVIGPQREEFPRDLSLI
SPLQAAHVVANPQAEGQLQWLNRRANALLANGVEL
RDNQLVVPSEGLYLIYSQVLFKGQGCPSTHVLLTH
TISRIAVSHQTKVNLLFAIKSPCQRETPEGAEAKP
WYEPIYLGGVFQLEKGDRLIAEINRPDYLYFAEYG
QVYFGIIALSSRAEGRGSLLTCGDVEENPGPMGMA
LPVTALLLPLALLLHAARPDIVLTQSPPSLAMSLG
KRATISCRASESVTILGSHLIHWYQQKPGQPPTLL
IQLASNVQTGVPARFSGSGSRTDFTLTIDPVEEDD
VAVYYCLQSRTIPRTFGGGTKLEIKGSTSGSGKPG
SGEGSTKGQIQLVQSGPELKKPGETVKISCKASGY
TFTDYSINWVKRAPGKGLKWMGWINTETREPAYAY
DFRGRFAFSLETSASTAYLQINNLKYEDTATYFCA
LDYSYAMDYWGQGTSVTVSSRTVTVSSQDPAEPKS
PDKTHTCPPCPAPELLGGPSVFLFPPKPKDTLMIS
RTPEVTCVVVDVSHEDPEVKFNWYVDGVEVHNAKT
KPREEQYNSTYRVVSVLTVLHQDWLNGKEYKCKVS
NKALPAPIEKTISKAKGQPREPQVYTLPPSRDELT
KNQVSLTCLVKGFYPSDIAVEWESNGQPENNYKTT
PPVLDSDGSFFLYSKLTVDKSRWQQGNVFSCSVMH
EALHNHYTQKSLSLSPGKKDPKFWVLVVVGGVLAC
YSLLVTVAFIIFWVRSKRSRLLHSDYMNMTPRRPG
PTRKHYQPYAPPRDFAAYRSRVKFSRSADAPAYQQ
GQNQLYNELNLGRREEYDVLDKRRGRDPEMGGKPR
RKNPQEGLYNELQKDKMAEAYSEIGMKGERRRGKG
HDGLYQGLSTATKDTYDALHMQALPPRGPQCTNYA
LLKLAGDVESNPGPMRISKPHLRSISIQCYLCLLL
NSHFLTEAGIHVFILGCFSAGLPKTEANWVNVISD
LKKIEDLIQSMHIDATLYTESDVHPSCKVTAMKCF
LLELQVISLESGDASIHDTVENLIILANNSLSSNG
NVTESGCKECEELEEKNIKEFLQSFVHIVQMFINT
S

Particular constructs that may be utilized have specific combinations of certain elements. In the present disclosure, the following constructs of BCMA1, BCMA2, BCMA3, BCMA4, and BCMA5 are utilized. Their elements are denoted below:

BCMA1 IgSPCOA7D12VLVH28Z15: Ig Heavy Chain Signal Peptide; codon optimized A7D12 light chain that is 5′ to codon optimized A7D12 heavy chain; CD28 costimulatory domain; CD3 zeta chain; and IL-15

BCMA2 CD8SPC11D53VLVH28Z15: CD8 signal peptide; non-codon optimized C11D5.3 light chain that is 5′ to non-codon optimized C11D5.3 heavy chain; IgG1 hinge; CD28 costimulatory domain; CD3 zeta endodomain; and IL-15

BCMA3 COGSPC11D53VLVHZIL15: GMSCF signal peptide; codon-optimized C11D5.3 light chain that is 5′ to codon-optimized C11D5.3 heavy chain; CD28 costimulatory domain; CD3 zeta; and IL-15

BCMA4 IgSPA7D12VHVL28Z15: Ig Heavy chain signal peptide; non codon-optimized A7D12 heavy chain that is 5′ to non codon-optimized A7D12 light chain; CD28 costimulatory domain; CD3 zeta; and IL-15

BCMA5 IgSPA7D12VLVH28Z15: Ig Heavy chain signal peptide; non codon-optimized A7D12 light chain that is 5′ to non codon-optimized A7D12 heavy chain; CD28 costimulatory domain; CD3 zeta; and IL-15

### Specific Examples of Construct Elements

Embodiments of certain examples of scFv sequences that target BCMA are provided below, including their respective VH chain, VL chain, and corresponding CDR sequences.

A7D12.2 scFv Sequences

One example of an A7D12.2 VL amino acid sequence is as follows:

(SEQ ID NO: 82)
D V V M T Q S H R F M S T S V G D R
V S I T C R A S Q D V N T A V S W
Y Q Q K P G Q S P K L L I F S A S Y
R Y T G V P D R F T G S G S G A D
F T L T I S S V Q A E D L A V Y Y C
Q Q H Y S T P W T F G G G T K L D
I K

One example of an A7D12.2 VL nucleic acid sequence is as follows:

(SEQ ID NO: 83)
GACGTGGTGATGACCCAGAGCCACAGGTTCATGAG
CACCAGCGTGGGCGACAGGGTGAGCATCACCTGCA
GGGCCAGCCAGGACGTGAACACCGCCGTGAGCTGG
TACCAGCAGAAGCCCGGCCAGAGCCCCAAGCTGCT
GATCTTCAGCGCCAGCTACAGGTACACCGGCGTGC
CCGACAGGTTCACCGGCAGCGGCAGCGGCGCCGAC
TTCACCCTGACCATCAGCAGCGTGCAGGCCGAGGA
CCTGGCCGTGTACTACTGCCAGCAGCACTACAGCA
CCCCCTGGACCTTCGGCGGCGGCACCAAGCTGGAC
ATCAAG

One example of an A7D12.2 VL CDR1 amino acid sequence is as follows:

(SEQ ID NO: 84)
R A S Q D V N T A V S

One example of an A7D12.2 VL CDR1 nucleic acid sequence is as follows:

(SEQ ID NO: 85)
AGGGCCAGCCAGGACGTGAACACCGCCGTGAGC

One example of an A7D12.2 VL CDR2 amino acid sequence is as follows:

(SEQ ID NO: 86)
SASYRYT

One example of an A7D12.2 VL CDR2 nucleic acid sequence is as follows:

(SEQ ID NO: 87)
AGCGCCAGCTACAGGTACACC

One example of an A7D12.2 VL CDR3 amino acid sequence is as follows:

(SEQ ID NO: 88)
Q Q H Y S T P W T

One example of an A7D12.2 VL CDR3 nucleic acid sequence is as follows:

(SEQ ID NO: 89)
CAGCAGCACTACAGCACCCCCTGGACC

An example of an A7D12.2 VH amino acid sequence is as follows:

(SEQ ID NO: 90)
Q I Q L V Q S G P D L K K P G E T V
K L S C K A S G Y T F T N F G M N
W V K Q A P G K G F K W M A W I N T
Y T G E S Y F A D D F K G R F A F
S V E T S A T T A Y L Q I N N L K T
E D T A T Y F C A R G E I Y Y G Y
D G G F A Y W G Q G T L V T V S A

An example of an A7D12.2 VH nucleic acid sequence is as follows:

(SEQ ID NO: 91)
CAGATCCAGCTGGTGCAGAGCGGCCCCGACCTGAA
GAAGCCCGGCGAGACCGTGAAGCTGAGCTGCAAGG
CCAGCGGCTACACCTTCACCAACTTCGGCATGAAC
TGGGTGAAGCAGGCCCCCGGCAAGGGCTTCAAGTG
GATGGCCTGGATCAACACCTACACCGGCGAGAGCT
ACTTCGCCGACGACTTCAAGGGCAGGTTCGCCTTC
AGCGTGGAGACCAGCGCCACCACCGCCTACCTGCA
GATCAACAACCTGAAGACCGAGGACACCGCCACCT
ACTTCTGCGCCAGGGGCGAGATCTACTACGGCTAC
GACGGCGGCTTCGCCTACTGGGGCCAGGGCACCCT
GGTGACCGTGAGCGCC

An example of an A7D12.2 VH CDR1 amino acid sequence is as follows:

(SEQ ID NO: 92)
N F G M N

An example of an A7D12.2 VH CDR1 nucleic acid sequence is as follows:

(SEQ ID NO: 93)
AACTTCGGCATGAAC

An example of an A7D12.2 VH CDR2 amino acid sequence is as follows:

(SEQ ID NO: 94)
I N T Y T G E S Y F A D D F K G

An example of an A7D12.2 VH CDR2 nucleic acid sequence is as follows:

(SEQ ID NO: 95)
ATCAACACCTACACCGGCGAGAGCTACTTCGCCGACGACTTCAAGGGC

An example of an A7D12.2 VH CDR3 amino acid sequence is as follows:

(SEQ ID NO: 96)
G E I Y Y G Y D G G F A Y

An example of an A7D12.2 VH CDR3 nucleic acid sequence is as follows:

(SEQ ID NO: 97)
GGCGAGATCTACTACGGCTACGACGGCGGCTTCGCCTAC

One example of Codon optimized A7D12.2 VH amino acid sequences is as follows:

(SEQ ID NO: 90)
Q I Q L V Q S G P D L K K P G E T V K L S C K A S
G Y T F T N F G M N W V K Q A P G K G F K W M A W
I N T Y T G E S Y F A D D F K G R F A F S V E T S
A T T A Y L Q I N N L K T E D T A T Y F C A R G E
I Y Y G Y D G G F A Y W G Q G T L V T V S A

One example of Codon optimized A7D12.2 VH nucleic acid sequences is as follows:

(SEQ ID NO: 98)
CAGATACAGCTCGTCCAATCCGGTCCCGATTTGAAAAAGCCTGGCGAAAC
AGTTAAACTGTCATGTAAGGCGAGCGGATACACGTTTACGAACTTCGGGA
TGAATTGGGTAAAACAGGCCCCGGGAAAAGGTTTTAAGTGGATGGCTTGG
ATAAACACCTACACTGGTGAGTCCTACTTCGCAGACGATTTCAAAGGGCG
GTTCGCGTTTTCAGTAGAGACTTCCGCCACAACTGCTTATCTCCAAATAA
ACAACTTGAAGACCGAGGATACGGCAACCTACTTTTGCGCTCGGGGCGAG
ATTTATTATGGATATGACGGCGGGTTCGCTTACTGGGGTCAGGGGACGTT
GGTTACCGTGTCTGCC

One example of Codon optimized A7D12.2 VH CDR1 amino acid sequence is as follows:

(SEQ ID NO: 92)
N F G M N

One example of Codon optimized A7D12.2 VH CDR1 nucleic acid sequence is as follows:

(SEQ ID NO: 99)
AACTTCGGGATGAAT

One example of Codon optimized A7D12.2 VH CDR2 amino acid sequence is as follows:

(SEQ ID NO: 94)
I N T Y T G E S Y F A D D F K G

One example of Codon optimized A7D12.2 VH CDR2 nucleic acid sequence is as follows:

(SEQ ID NO: 100)
ATAAACACCTACACTGGTGAGTCCTACTTCGCAGACGATTTCAAAGGG

One example of Codon optimized A7D12.2 VH CDR3 amino acid sequence is as follows:

(SEQ ID NO: 96)
G E I Y Y G Y D G G F A Y

One example of Codon optimized A7D12.2 VH CDR3 nucleic acid sequence is as follows:

(SEQ ID NO: 101)
GGCGAGATTTATTATGGATATGACGGCGGGTTCGCTTAC

An example of Codon optimized A7D12.2 VL amino acid sequence is as follows:

(SEQ ID NO: 82)
D V V M T Q S H R F M S T S V G D R V S I T C R A
S Q D V N T A V S W Y Q Q K P G Q S P K L L I F S
A S Y R Y T G V P D R F T G S G S G A D F T L T I
S S V Q A E D L A V Y Y C Q Q H Y S T P W T F G G
G T K L D I K

An example of Codon optimized A7D12.2 VL nucleic acid sequence is as follows:

(SEQ ID NO: 102)
ACGTGGTGATGACGCAGAGCCACCGATTCATGAGTACCTCTGTAGGCGAC
CGCGTCTCAATTACTTGTCGAGCGTCTCAGGACGTAAATACAGCGGTGAG
CTGGTATCAGCAAAAGCCCGGACAGAGCCCGAAATTGCTGATCTTTTCAG
CCTCATACAGATATACCGGAGTCCCAGACCGCTTTACAGGTTCCGGTAGT
GGCGCGGACTTTACTCTCACAATCAGCTCTGTACAAGCTGAAGATTTGGC
TGTTTACTATTGTCAGCAGCACTATAGTACGCCCTGGACCTTCGGGGGCG
GTACGAAGTTGGATATTAAG

One example of codon optimized A7D12.2 VL CDR1 amino acid sequence is as follows:

(SEQ ID NO: 84)
R A S Q D V N T A V S

One example of codon optimized A7D12.2 VL CDR1 nucleic acid sequence is as follows:

(SEQ ID NO: 103)
CGAGCGTCTCAGGACGTAAATACAGCGGTGAGC

One example of codon optimized A7D12.2 VL CDR2 amino acid sequence is as follows:

(SEQ ID NO: 86)
SASYRYT

One example of codon optimized A7D12.2 VL CDR2 nucleic acid sequence is as follows:

(SEQ ID NO: 104)
TCAGCCTCATACAGATATACC

An example of codon optimized A7D12.2 VL CDR3 amino acid sequence is as follows:

(SEQ ID NO: 88)
QQHYSTPWT

An example of codon optimized A7D12.2 VL CDR3 nucleic acid sequence is as follows:

(SEQ ID NO: 105)
CAGCAGCACTATAGTACGCCCTGGACC

C11D5.3 scFv Sequences

An example of C11D5.3 VL chain amino acid sequence is as follows:

(SEQ ID NO: 106)
DIVLTQSPPSLAMSLGKRATISCRASESVTILGSHLIHWYQ
QKPGQPPTLLIQLASNVQTGVPARFSGSGSRTDFTLTIDPVEED
DVAVYYCLQSRTIPRTFGGGTKLEIK

An example of C11D5.3 VL chain nucleic acid sequence is as follows:

(SEQ ID NO: 107)
GACATCGTGCTGACCCAGAGCCCCCCCAGCCTGGCCATGTCTCTGGGCAA
GAGAGCCACCATCAGCTGCCGGGCCAGCGAGAGCGTGACCATCCTGGGCA
GCCACCTGATCCACTGGTATCAGCAGAAGCCCGGCCAGCCCCCCACCCTG
CTGATCCAGCTCGCCAGCAATGTGCAGACCGGCGTGCCCGCCAGATTCAG
CGGCAGCGGCAGCAGAACCGACTTCACCCTGACCATCGACCCCGTGGAAG
AGGACGACGTGGCCGTGTACTACTGCCTGCAGAGCCGGACCATCCCCCGG
ACCTTTGGCGGAGGCACCAAACTGGAAATCAAG

An example of C11D5.3 VL chain CDR1 amino acid sequence is as follows:

(SEQ ID NO: 108)
RASESVTILGSHLIH

An example of C11D5.3 VL chain CDR1 nucleic acid sequence is as follows:

(SEQ ID NO: 109)
CGGGCCAGCGAGAGCGTGACCATCCTGGGCAGCCACCTGATCCAC

An example of C11D5.3 VL chain CDR2 amino acid sequence is as follows:

(SEQ ID NO: 110)
LASNVQT

An example of C11D5.3 VL chain CDR2 nucleic acid sequence is as follows:

(SEQ ID NO: 111)
CTCGCCAGCAATGTGCAGACC

One example of C11D5.3 VL chain CDR3 amino acid sequence is as follows:

(SEQ ID NO: 112)
LQSRTIPRT

One example of C11D5.3 VL chain CDR3 nucleic acid sequence is as follows:

(SEQ ID NO: 113)
CTGCAGAGCCGGACCATCCCCCGGACC

One example of C11D5.3 VH chain amino acid sequence is as follows:

(SEQ ID NO: 114)
QIQLVQSGPELKKPGETVKISCKASGYTFTDYSINWVKRA
PGKGLKWMGWINTETREPAYAYDFRGRFAFSLETSASTAYLQI
NNLKYEDTATYFCALDYSYAMDYWGQGTSVTVSS

One example of C11D5.3 VH chain nucleic acid sequence is as follows:

(SEQ ID NO: 115)
CAGATTCAGCTGGTGCAGAGCGGCCCTGAGCTGAAGAAACCCGGCGAGA
CAGTGAAGATCAGCTGCAAGGCCTCCGGCTACACCTTCACCGACTACAG
CATCAACTGGGTGAAAAGAGCCCCTGGCAAGGGCCTGAAGTGGATGGGC
TGGATCAACACCGAGACAAGAGAGCCCGCCTACGCCTACGACTTCCGGG
GCAGATTCGCCTTCAGCCTGGAAACCAGCGCCAGCACCGCCTACCTGCA
GATCAACAACCTGAAGTACGAGGACACCGCCACCTACTTTTGCGCCCTG
GACTACAGCTACGCTATGGACTACTGGGGCCAGGGCACCAGCGTGACCG
TGTCCAGC

One example of C11D5.3 VH chain CDR1 amino acid sequence is as follows:

(SEQ ID NO: 116)
DYSIN

One example of C11D5.3 VH chain CDR1 nucleic acid sequence is as follows:

(SEQ ID NO: 117)
CTGATGTCGTAGTTG

One example of C11D5.3 VH chain CDR2 amino acid sequence is as follows:

(SEQ ID NO: 118)
WINTETREPAYAYDFRG

One example of C11D5.3 VH chain CDR2 nucleic acid sequence is as follows:

(SEQ ID NO: 119)
TGGATCAACACCGAGACAAGAGAGCCCGCCTACGCCTACGACTTCCGGGGC

One example of C11D5.3 VH chain CDR3 amino acid sequence is as follows:

(SEQ ID NO: 120)
DYSYAMDY

One example of C11D5.3 VH chain CDR3 nucleic acid sequence is as follows:

(SEQ ID NO: 121)
GACTACAGCTACGCTATGGACTAC

An example of codon optimized C11D5.3 VL chain amino acid sequence is as follows:

(SEQ ID NO: 122)
DIVLTQSPPSLAMSLGKRATISCRASESVTILGSHLIHWYQ
QKPGQPPTLLIQLASNVQTVPARFSGSGSRTDFTLTIDPVEEDD
VAVYYCLQSRTIPRTFGGGTKLEIK

An example of codon optimized C11D5.3 VL chain nucleic acid sequence is as follows:

(SEQ ID NO: 123)
GACATTGTTTTGACCCAATCACCTCCCTCTCTCGCCATGTCCTTGGGTA
AACGGGCAACAATCTCCTGTAGAGCTTCCGAAAGTGTAACAATTCTTGG
AAGCCACCTCATACATTGGTATCAGCAAAAGCCGGGGCAGCCCCCTACA
TTGCTCATTCAGTTGGCTTCAAATGTCCAGACGGGTGTACCAGCGAGAT
TCTCAGGGAGTGGCTCCCGAACGGATTTCACACTGACGATTGATCCCGT
CGAAGAGGACGATGTCGCAGTTTATTATTGCCTCCAAAGTCGGACAATT
CCGAGGACTTTTGGAGGCGGAACAAAATTGGAAATCAAA

An example of codon optimized C11D5.3 VL chain CDR1 amino acid sequence is as follows:

(SEQ ID NO: 108)
RASESVTILGSHLIH

An example of codon optimized C11D5.3 VL chain CDR1 nucleic acid sequence is as follows:

(SEQ ID NO: 124)
AGAGCTTCCGAAAGTGTAACAATTCTTGGAAGCCACCTCATACAT

An example of codon optimized C11D5.3 VL chain CDR2 amino acid sequence is as follows:

(SEQ ID NO: 110)
LASNVQT

An example of codon optimized C11D5.3 VL chain CDR2 nucleic acid sequence is as follows:

(SEQ ID NO: 125)
TTGGCTTCAAATGTCCAGACGG

An example of codon optimized C11D5.3 VL chain CDR3 amino acid sequence is as follows:

(SEQ ID NO: 112)
LQSRTIPRT

An example of codon optimized C11D5.3 VL chain CDR3 nucleic acid sequence is as follows:

(SEQ ID NO: 126)
CTCCAAAGTCGGACAATTCCGAGGACT

An example of codon optimized C11D5.3 VH chain amino acid sequence is as follows:

(SEQ ID NO: 127)
QIQLVQSGPELKKPGETVKISCKASGYTFTDYSINWVKRA
PGKGLKWMGWINTETREPAYAFDFRGRFAFSLETSASTAYLQI
NNLKYEDTATYFCALDYSYAMDYWGQGTSVTVSS

An example of codon optimized C11D5.3 VH chain nucleic acid sequence is as follows:

(SEQ ID NO: 128)
CAAATCCAGCTCGTCCAATCCGGTCCAGAGTTGAAGAAACCCGGCGAGAC
GGTAAAAATCAGCTGTAAAGCCTCAGGTTACACGTTTACGGACTATAGCA
TTAATTGGGTTAAGAGGGCTCCGGGGAAGGGGCTCAAATGGATGGGCTGG
ATAAACACAGAGACGAGAGAGCCCGCATATGCGTTCGACTTTAGAGGTCG
ATTCGCTTTCAGTCTTGAAACCTCTGCTTCTACCGCGTATCTCCAGATAA
ACAACCTGAAATATGAGGATACAGCAACTTATTTTTGCGCTCTCGATTAC
AGCTATGCGATGGATTATTGGGGACAAGGAACTTCCGTGACTGTGTCAAG
C

An example of codon optimized C11D5.3 VH chain CDR1 amino acid sequence is as follows:

| (SEQ ID NO: 116) | |
|---|---|
| | D Y S I N |

An example of codon optimized C11D5.3 VH chain CDR1 nucleic acid sequence is as follows:

| (SEQ ID NO: 129) | |
|---|---|
| | GACTATAGCATTAAT |

An example of codon optimized C11D5.3 VH chain CDR2 amino acid sequence is as follows:

| (SEQ ID NO: 130) | |
|---|---|
| | W I N T E T R E P A Y A F D F R G |

An example of codon optimized C11D5.3 VH chain CDR2 nucleic acid sequence is as follows:

(SEQ ID NO: 131)
TGGATAAACACAGAGACGAGAGAGCCCGCATATGCGTTCGACTTTAGAGG
T

An example of codon optimized C11D5.3 VH chain CDR3 amino acid sequence is as follows:

| (SEQ ID NO: 120) | |
|---|---|
| | D Y S Y A M D Y |

An example of codon optimized C11D5.3 VH chain CDR3 nucleic acid sequence is as follows:

| (SEQ ID NO: 132) | |
|---|---|
| | GATTACAGCTATGCGATGGATTAT |

C12A3.2 scFv Sequences

An example of codon optimized C12A3.2 VL chain amino acid sequence is as follows:

(SEQ ID NO: 133)
D I V L T Q S P P S L A M S L G K R A T I S C R A
S E S V T I L G S H L I Y W Y Q Q K P G Q P P T L
L I Q L A S N V Q T G V P A R F S G S G S R T D F
T L T I D P V E E D D V A V Y Y C L Q S R T I P R
T F G G G T K L E I K

An example of codon optimized C12A3.2 VL chain nucleic acid sequence is as follows:

(SEQ ID NO: 134)
GACATCGTGCTGACCCAGAGCCCCCCCAGCCTGGCCATGTCTCTGGGCAA
GAGAGCCACCATCAGCTGCCGGGCCAGCGAGAGCGTGACCATCCTGGGCA
GCCACCTGATCTACTGGTATCAGCAGAAGCCTGGCCAGCCCCCCACCCTG
CTGATCCAGCTGGCTAGCAATGTGCAGACCGGCGTGCCCGCCAGATTCAG
CGGCAGCGGCAGCAGAACCGACTTCACCCTGACCATCGACCCCGTGGAAG
AGGACGACGTGGCCGTGTACTACTGCCTGCAGAGCCGGACCATCCCCCGG
ACCTTTGGCGGAGGAACAAAGCTGGAAATCAAG

An example of codon optimized C12A3.2 VL CDR1 amino acid sequence is as follows:

| (SEQ ID NO: 135) | |
|---|---|
| | A S E S V T I L G S H L I Y |

An example of codon optimized C12A3.2 VL CDR1 nucleic acid sequence is as follows:

| (SEQ ID NO: 136) | |
|---|---|
| | CCAGCGAGAGCGTGACCATCCTGGGCAGCCACCTGATCTAC |

One example of codon optimized C12A3.2 VL CDR2 amino acid sequence is as follows:

| (SEQ ID NO: 137) | |
|---|---|
| | A S N V Q T |

One example of codon optimized C12A3.2 VL CDR2 nucleic acid sequence is as follows:

| (SEQ ID NO: 138) | |
|---|---|
| | GCTAGCAATGTGCAGACC |

An example of codon optimized C12A3.2 VL CDR3 amino acid sequence is as follows:

| (SEQ ID NO: 139) | |
|---|---|
| | L Q S R T I P R T |

An example of codon optimized C12A3.2 VL CDR3 nucleic acid sequence is as follows:

| (SEQ ID NO: 140) | |
|---|---|
| | CTGCAGAGCCGGACCATCCCCCGGACC |

An example of codon optimized C12A3.2 VH chain amino acid sequence is as follows:

(SEQ ID NO: 141)
Q I Q L V Q S G P E L K K P G E T V K I S C K A S
G Y T F R H Y S M N W V K Q A P G K G L K W M G R
I N T E S G V P I Y A D D F K G R F A F S V E T S
A S T A Y L V I N N L K D E D T A S Y F C S N D Y
L Y S L D F W G Q G T A L T V S S

An example of codon optimized C12A3.2 VH chain nucleic acid sequence is as follows:

(SEQ ID NO: 142)
CAGATTCAGCTGGTGCAGAGCGGCCCTGAGCTGAAGAAACCCGGCGAGAC
AGTGAAGATCAGCTGCAAGGCCTCCGGCTACACCTTCCGGCACTACAGCA
TGAACTGGGTGAAACAGGCCCCTGGCAAGGGCCTGAAGTGGATGGGCCGG
ATCAACACCGAGAGCGGCGTGCCCATCTACGCCGACGACTTCAAGGGCAG
ATTCGCCTTCAGCGTGGAAACCAGCGCCAGCACCGCCTACCTGGTGATCA
ACAACCTGAAGGACGAGGATACCGCCAGCTACTTCTGCAGCAACGACTAC
CTGTACAGCCTGGACTTCTGGGGCCAGGGCACCGCCCTGACCGTGTCCAG
C

One example of codon optimized C12A3.2 VH CDR1 amino acid sequence is as follows:

(SEQ ID NO: 143)
H Y S M N

One example of codon optimized C12A3.2 VH CDR1 nucleic acid sequence is as follows:

(SEQ ID NO: 144)
CACTACAGCATGAAC

An example of codon optimized C12A3.2 VH CDR2 amino acid sequence is as follows:

(SEQ ID NO: 145)
R I N T E S G V P I Y A D D F K G

An example of codon optimized C12A3.2 VH CDR2 nucleic acid sequence is as follows:

(SEQ ID NO: 146)
CGGATCAACACCGAGAGCGGCGTGCCCATCTACGCCGACGACTTCAAGGG
C

An example of codon optimized C12A3.2 VH CDR3 amino acid sequence is as follows:

(SEQ ID NO: 147)
Y L Y S L D F

An example of codon optimized C12A3.2 VH CDR3 nucleic acid sequence is as follows:

(SEQ ID NO: 148)
TACCTGTACAGCCTGGACTTC

### II. SUICIDE GENES

In particular embodiments, a suicide gene is utilized in conjunction with cell therapy of any kind to control its use and allow for termination of the cell therapy at a desired event and/or time. The suicide gene is employed in transduced cells for the purpose of eliciting death for the transduced cells when needed. The cells of the present disclosure that have been modified to harbor a vector encompassed by the disclosure may comprise one or more suicide genes. In some embodiments, the term “suicide gene” as used herein is defined as a gene which, upon administration of a prodrug or other agent, effects transition of a gene product to a compound which kills its host cell. In other embodiments, a suicide gene encodes a gene product that is, when desired, targeted by an agent (such as an antibody) that targets the suicide gene product.

Examples of suicide gene/prodrug combinations which may be used are Herpes Simplex Virus-thymidine kinase (HSV-tk) and ganciclovir, acyclovir, or FIAU; oxidoreductase and cycloheximide; cytosine deaminase and 5-fluorocytosine; thymidine kinase thymidilate kinase (Tdk::Tmk) and AZT; and deoxycytidine kinase and cytosine arabinoside. The E. coli purine nucleoside phosphorylase, a so-called suicide gene that converts the prodrug 6-methylpurine deoxyriboside to toxic purine 6-methylpurine, may be used. Other examples of suicide genes used with prodrug therapy are the E. coli cytosine deaminase gene and the HSV thymidine kinase gene.

Exemplary suicide genes also include CD20, CD52, EGFRv3, or inducible caspase 9. In one embodiment, a truncated version of EGFR variant III (EGFRv3) may be used as a suicide antigen that can be ablated by Cetuximab. Further suicide genes known in the art that may be used in the present disclosure include Purine nucleoside phosphorylase (PNP), Cytochrome p450 enzymes (CYP), Carboxypeptidases (CP), Carboxylesterase (CE), Nitroreductase (NTR), Guanine Ribosyltransferase (XGRTP), Glycosidase enzymes, Methionine-α,γ-lyase (MET), and Thymidine phosphorylase (TP).

In particular embodiments, vectors that encode the BCMA-targeting CAR, or any vector in a NK cell encompassed herein, include one or more suicide genes. The suicide gene may or may not be on the same vector as a BCMA-targeting CAR. In cases wherein the suicide gene is present on the same vector as the BCMA-targeting CAR, the suicide gene and the CAR may be separated by an IRES or 2A element, for example.

In specific embodiments, the suicide gene is a tumor necrosis factor (TNF)-alpha mutant that is uncleavable by standard enzymes that cleave TNF in nature, such as TNF-alpha-converting enzyme (also referred to as TACE). As such, the TNF-alpha mutant is membrane-bound and nonsecretable, in particular embodiments. The TNF-alpha mutant used in the disclosure is targetable by one or more agents that bind the mutant, including at least an antibody, such that following binding of the agent(s) to the TNF-alpha mutant on the surface of the cell, the cell dies. Embodiments of the disclosure allow the TNF-alpha mutant to be utilized as a marker for cells that express it.

Cells expressing the uncleavable TNF-alpha mutants can be targeted for selective deletion including, for example, using FDA-approved TNF-α antibodies currently in the clinic, such as etanercept, infliximab or adalilumab. The mutated TNF-alpha polypeptide may be co-expressed with one or more therapeutic transgenes in the cell, such as a gene encoding a TCR or CAR, including BCMA-targeting TCRs and/or CARs. In addition, the TNF-alpha mutant expressing cells have superior activity against the tumor target, mediated by the biological activity of the membrane-bound TNF-alpha protein.

With respect to wild-type, TNF-alpha has a 26 kD transmembrane form and a 17 kD secretory component. Some mutants described in Perez et al. (1990) may be utilized in the disclosure. In specific embodiments, examples of TNF-alpha mutants of the disclosure include at least the following with respect to the 17 kD TNF: (1) deletion of Val1 and deletion of Pro112; (2) deletion of Val13; (3) deletion of Val1 and deletion of Val13; (4) deletion of Val1 through and including Pro112 and deletion of Val13 (delete 13aa); (5) deletion of Ala-3 through to and including Val 13 (delete 14 aa). In specific embodiments, a TNF-alpha mutant comprises deletion of the respective amino acid at position −3, −2, −1, 1, 2, 3, 4, 5, 6, 7, 8, 9, 10, 11, 12, 13, or a combination thereof. Specific combinations include deletions at positions −3 through and including 13; −3 through and including 12; −3 through and including 11; −3 through and including 10; −3 through and including 9; −3 through and including 8; −3 through and including 7; −3 through and including 6; −3 through and including 5; −3 through and including 4; −3 through and including 3; −3 through and including 2; −3 through and including 1; −3 through and including −1; −3 through and including −2; −2 through and including 13; −2 through and including 12; −2 through and including 11; −2 through and including 10; −2 through and including 9; −2 through and including 8; −2 through and including 7; −2 through and including 6; −2 through and including 5; −2 through and including 4; −2 through and including 3; −2 through and including 2; −2 through and including 1; −2 through and including −1; −1 through and including 13; −1 through and including 12; −1 through and including 11; −1 through and including 10; −1 through and including 9; −1 through and including 8; −1 through and including 7; −1 through and including 6; −1 through and including 5; −1 through and including 4; −1 through and including 3; −1 through and including 2; −1 through and including 1; 1 through and including 13; 1 through and including 12; 1 through and including 11; 1 through and including 10; 1 through and including 9; 1 through and including 8; 1 through and including 7; 1 through and including 6; 1 through and including 5; 1 through and including 4; 1 through and including 3; 1 through and including 2; and so forth.

The TNF-alpha mutants may be generated by any suitable method, but in specific embodiments they are generated by site-directed mutagenesis. In some cases, the TNF-alpha mutants may have mutations other than those that render the protein uncleavable. In specific cases, the TNF-alpha mutants may have 1, 2, 3, or more mutations other than the deletions at Val1, Pro12, and/or Val13 or the region there between. The mutations other than those that render the mutants nonsecretable may be one or more of an amino acid substitution, deletion, addition, inversion, and so forth. In cases wherein the additional mutation is an amino acid substitution, the substitution may or may not be to a conservative amino acid, for example. In some cases, 1, 2, 3, 4, 5, or more additional amino acids may be present on the N-terminal and/or C-terminal ends of the protein. In some cases, a TNF-alpha mutant has (1) one or more mutations that render the mutant nonsecretable; (2) one or more mutations that prevents outside-in signaling for the mutant; and/or (3) one or more mutations that interfere with binding of the mutant to TNF Receptor 1 and/or TNF Receptor 2.

In particular embodiments, the TNF-alpha mutant polypeptide comprises a deletion with respect to SEQ ID NO:30 of the following: amino acid residue 1 and amino acid residue 12; amino acid residue 1 and amino acid residue 13; amino acid residues 1-12; amino acid residues 1-13; or amino acid residues −1 to 13.

TNF-alpha delVal1 delPro112 amino acid sequence:

(SEQ ID NO: 20)
MSTESMIRDVELAEEALPKKTGGPQGSRRCLFLSLFSFLIVAGATTLFCL
LHFGVIGPQREEFPRDLSLISPLAQARSSSRTPSDKVAHVVANPQAEGQL
QWLNRRANALLANGVELRDNQLVVPSEGLYLIYSQVLFKGQGCPSTHVLL
THTISRIAVSYQTKVNLLSAIKSPCQRETPEGAEAKPWYEPIYLGGVFQL
EKGDRLSAEINRPDYLDFAESGQVYFGIIAL

TNF-alpha mutant-delVal1 del Pro112 nucleic acid sequence

(SEQ ID NO: 21)
atgagcactgaaagcatgatccgggacgtggagctggccgaggaggcgct
ccccaagaagacaggggggccccagggctccaggcggtgcttgttcctca
gcctcttctccttcctgatcgtggcaggcgccaccacgctcttctgcctg
ctgcactttggagtgatcggcccccagagggaagagttccccagggacct
ctctctaatcagccctctggcccaggcaagatcatcttctcgaaccccga
gtgacaaggtagcccatgttgtagcaaaccctcaagctgaggggcagctc
cagtggctgaaccgccgggccaatgccctcctggccaatggcgtggagct
gagagataaccagctggtggtgccatcagagggcctgtacctcatctact
cccaggtcctcttcaagggccaaggctgcccctccacccatgtgctcctc
acccacaccatcagccgcatcgccgtctcctaccagaccaaggtcaacct
cctctctgccatcaagagcccctgccagagggagaccccagagggggctg
aggccaagccctggtatgagcccatctatctgggaggggtcttccagctg
gagaagggtgaccgactcagcgctgagatcaatcggcccgactatctcga
ctttgccgagtctgggcaggtctactttgggatcattgccctgtcg

TNFa mutant-del Val1 to Val13 amino acid sequence (delete 13aa)

(SEQ ID NO: 22)
MSTESMIRDVELAEEALPKKTGGPQGSRRCLFLSLFSFLIVAGATTLFCL
LHFGVIGPQREEFPRDLSLISPLAQAAHVVANPQAEGQLQWLNRRANALL
ANGVELRDNQLVVPSEGLYLIYSQVLFKGQGCPSTHVLLTHTISRIAVSY
QTKVNLLSAIKSPCQRETPEGAEAKPWYEPIYLGGVFQLEKGDRLSAEIN
RPDYLDFAESGQVYFGIIAL

TNFa mutant-del Val1 to Pro112 delVal13 (delete 13 aa) nucleic acid sequence:

(SEQ ID NO: 23)
atgagcactgaaagcatgatccgggacgtggagctggccgaggaggcgct
ccccaagaagacaggggggccccagggctccaggcggtgcttgttcctca
gcctcttctccttcctgatcgtggcaggcgccaccacgctcttctgcctg
ctgcactttggagtgatcggcccccagagggaagagttccccagggacct
ctctctaatcagccctctggcccaggcagcccatgttgtagcaaaccctc
aagctgaggggcagctccagtggctgaaccgccgggccaatgccctcctg
gccaatggcgtggagctgagagataaccagctggtggtgccatcagaggg
cctgtacctcatctactcccaggtcctcttcaagggccaaggctgcccct
ccacccatgtgctcctcacccacaccatcagccgcatcgccgtctcctac
cagaccaaggtcaacctcctctctgccatcaagagcccctgccagaggga
gaccccagagggggctgaggccaagccctggtatgagcccatctatctgg
gaggggtcttccagctggagaagggtgaccgactcagcgctgagatcaat
cggcccgactatctcgactttgccgagtctgggcaggtctactttgggat
cattgccctgtcg

TNF-alpha delVal1 delVal13 amino acid sequence:

(SEQ ID NO: 24)
MSTESMIRDVELAEEALPKKTGGPQGSRRCLFLSLFSFLIVAGATTLFCL
LHFGVIGPQREEFPRDLSLISPLAQARSSSRTPSDKPAHVVANPQAEGQL
QWLNRRANALLANGVELRDNQLVVPSEGLYLIYSQVLFKGQGCPSTHVLL
THTISRIAVSYQTKVNLLSAIKSPCQRETPEGAEAKPWYEPIYLGGVFQL
EKGDRLSAEINRPDYLDFAESGQVYFGIIAL

TNF-alpha delVal1 delVal13 nucleic acid sequence:

(SEQ ID NO: 25)
atgagcactgaaagcatgatccgggacgtggagctggccgaggaggcgct
ccccaagaagacaggggggccccagggctccaggcggtgcttgttcctca
gcctcttctccttcctgatcgtggcaggcgccaccacgctcttctgcctg
ctgcactttggagtgatcggcccccagagggaagagttccccagggacct
ctctctaatcagccctctggcccaggcaagatcatcttctcgaaccccga
gtgacaagcctgcccatgttgtagcaaaccctcaagctgaggggcagctc
cagtggctgaaccgccgggccaatgccctcctggccaatggcgtggagct
gagagataaccagctggtggtgccatcagagggcctgtacctcatctact
cccaggtcctcttcaagggccaaggctgcccctccacccatgtgctcctc
acccacaccatcagccgcatcgccgtctcctaccagaccaaggtcaacct
cctctctgccatcaagagcccctgccagagggagaccccagagggggctg
aggccaagccctggtatgagcccatctatctgggaggggtcttccagctg
gagaagggtgaccgactcagcgctgagatcaatcggcccgactatctcga
ctttgccgagtctgggcaggtctactttgggatcattgccctgtcg

TNF-alpha delAla-3 to Val 13 nucleic acid sequence:

(SEQ ID NO: 26)
TCGAGTCGAGATGAGCACTGAAAGCATGATCCGGGACGTGGAGCTGGCCG
AGGAGGCGCTCCCCAAGAAGACAGGGGGGCCCCAGGGCTCCAGGCGGTGC
TTGTTCCTCAGCCTCTTCTCCTTCCTGATCGTGGCAGGCGCCACCACGCT
CTTCTGCCTGCTGCACTTTGGAGTGATCGGCCCCCAGAGGGAAGAGTTCC
CCAGGGACCTCTCTCTAATCAGCCCTCTGCAGGCAGCCCATGTTGTAGCA
AACCCTCAAGCTGAGGGGCAGCTCCAGTGGCTGAACCGCCGGGCCAATGC
CCTCCTGGCCAATGGCGTGGAGCTGAGAGATAACCAGCTGGTGGTGCCAT
CAGAGGGCCTGTACCTCATCTACTCCCAGGTCCTCTTCAAGGGCCAAGGC
TGCCCCTCCACCCATGTGCTCCTCACCCACACCATCAGCCGCATCGCCGT
CTCCTACCAGACCAAGGTCAACCTCCTCTCTGCCATCAAGAGCCCCTGCC
AGAGGGAGACCCCAGAGGGGGCTGAGGCCAAGCCCTGGTATGAGCCCATC
TATCTGGGAGGGGTCTTCCAGCTGGAGAAGGGTGACCGACTCAGCGCTGA
GATCAATCGGCCCGACTATCTCgACTTTGCCGAGTCTGGGCAGGTCTACT
TTGGGATCATTGCCCTGTCGTCG

TNF-alpha delAla-3 to Val 13 amino acid sequence:

(SEQ ID NO: 27)
MSTESMIRDVELAEEALPKKTGGPQGSRRCLFLSLFSFLIVAGATTLFC
LLHFGVIGPQREEFPRDLSLISPLQAAHVVANPQAEGQLQWLNRRANAL
LANGVELRDNQLVVPSEGLYLIYSQVLFKGQGCPSTHVLLTHTISRIAV
SYQTKVNLLSAIKSPCQRETPEGAEAKPWYEPIYLGGVFQLEKGDRLSA
EINRPDYLDFAESGQVYFGIIAL

Embodiments of the disclosure include TNF-alpha mutants with del Ala-3 to Val13 nucleic acid sequence in addition to an example of a CIK motif mutation that prevents outside-in signaling and/or other mutations that interfere with TNF-alpha binding to TNF Receptor 1 and TNF Receptor 2

(SEQ ID NO: 28)
ATGAGCACTGAAATGCATCCCGGAAGGGGGTCCTGGCACGAGGAGGCGC
TCCCCAAGAAGACAGGGGGGCCCCAGGGCTCCAGGCGGTGCTTGTTCCT
CAGCCTCTTCTCCTTCCTGATCGTGGCAGGCGCCACCACGCTCTTCTTC
CTGCTGCACTTTGGAGTGATCGGCCCCCAGAGGGAAGAGTTCCCCAGGG
ACCTCTCTCTAATCAGCCCTCTGGCCCATGTTGTAGCAAACCCTCAAGC
TGAGGGGCAGCTCCAGTGGCTGAACCGCCGGGCCAATGCCCTCCTGGCC
AATGGCGTGGAGCTGAGAGATAACCAGCTGGTGGTGCCATCAGAGGGCC
TGTACCTCATCTACTCCCAGGTCCTCTTCAAGGGCCAAGGCTGCCCCTC
CACCCATGTGCTCCTCACCCACACCATCAGCCGCATCGCCGTCTCCCAC
CAGACCAAGGTCAACCTCCTCTTCGCCATCAAGAGCCCCTGCCAGAGGG
AGACCCCAGAGGGGGCTGAGGCCAAGCCCTGGTATGAGCCCATCTATCT
GGGAGGGGTCTTCCAGCTGGAGAAGGGTGACCGACTCATCGCTGAGATC
AATCGGCCCGACTATCTCTACTTTGCCGAGTATGGGCAGGTCTACTTTG
GGATCATTGCCCTGTCG

TNF-alpha mutant with del Ala-3 to Val13 amino acid sequence encoded by SEQ ID NO:28

(SEQ ID NO: 29)
MSTEMHPGRGSWHEEALPKKTGGPQGSRRCLFLSLFSFLIVAGATTLFF
LLHFGVIGPQREEFPRDLSLISPLAHVVANPQAEGQLQWLNRRANALLA
NGVELRDNQLVVPSEGLYLIYSQVLFKGQGCPSTHVLLTHTISRIAVSH
QTKVNLLFAIKSPCQRETPEGAEAKPWYEPIYLGGVFQLEKGDRLIAEI
NRPDYLYFAEYGQVYFGIIALS

In specific embodiments, a TNF-alpha mutant may comprise deletion of Ala-3 to Val13 but not also comprise a CIK motif mutation and a mutation that interferes with binding to TNF Receptor 1 and/or TNF Receptor 2.

TNF Wild type, 26 kD, version amino acid sequence:

(SEQ ID NO: 30)
MSTESMIRDVELAEEALPKKTGGPQGSRRCLFLSLFSFLIVAGATTLFC
LLHFGVIGPQREEFPRDLSLISPLAQAVRSSSRTPSDKPVAHVVANPQA
EGQLQWLNRRANALLANGVELRDNQLVVPSEGLYLIYSQVLFKGQGCPS
THVLLTHTISRIAVSYQTKVNLLSAIKSPCQRETPEGAEAKPWYEPIYL
GGVFQLEKGDRLSAEINRPDYLDFAESGQVYFGIIAL

TNF Wild type, 17 kD version, amino acid sequence

(SEQ ID NO: 31)
VRSSSRTPSDKPVAHVVANPQAEGQLQWLNRRANALLANGVELRDNQLV
VPSEGLYLIYSQVLFKGQGCPSTHVLLTHTISRIAVSYQTKVNLLSAIK
SPCQRETPEGAEAKPWYEPIYLGGVFQLEKGDRLSAEINRPDYLDFAES
GQVYFGIIAL

TNF-alpha mutants lacking intracellular TNF signaling or TNF-receptor binding capability

These TNF-alpha mutants lacking intracellular TNF signaling or TNF-receptor binding capability mutants have mutations in the cytoplasmic signaling domain and/or in the TNF-receptor binding regions and therefore do not exert any biological activity as they lack reverse signaling capability and/or the ability to bind TNF-receptors, respectively. This allows for the TNF-alpha in the construct to be a target for TNF inhibitors, while exerting no biological activity.

In some embodiments of the disclosure, TNF-alpha mutants lack part or all of the intracytoplasmic domain of TNF-alpha such that the TNF-alpha mutant is unable to exert intracellular signaling (reverse signaling). The nonsecretable TNF-alpha mutants may or may not also be mutated to lack part or all of the intracytoplasmic domain.

Any aspect of TNF-alpha may be mutated, regardless of whether or not the mutation would render the TNF-alpha to be nonsecretable. As an example, and with respect to the structure of TNF-alpha, any of the following regions of TNF-alpha may be mutated. The intracytoplasmic domain comprises MSTESMIRDVELAEEALPKKTGGPQGSRRCLFL (SEQ ID NO:32). The casein kinase I (CKI) site is STES (SEQ ID NO:33). The transmembrane domain is FSFLIVAGATTLFCLLHFGVI (SEQ ID NO:34). The SPPL2b cut site is SL/LI. The linker comprises GPQREEFPRDLSLISPLAQA (SEQ ID NO:35). The TACE cute site is VRSSSRTPSDKPV (SEQ ID NO:36). P01375 refers to the UniProt number of the protein.

Specific examples of TNF-alpha mutant for the del Ala-1 to del13 CKI motif mutated sequence underlined) for nucleic acid and amino acid, respectively, is as follows:

(SEQ ID NO: 37)
atgagcactgaaaTGCATCCCGGAAGGGGGTCCTGGCACgaggaggcgctccccaagaagacaggg
gggccccagggctccaggcggtgcttgttcctcagcctcttctccttcctgatcgtggcaggcgccaccacgctcttctgcctgctgcactttg
gagtgatcggcccccagagggaagagttccccagggacctctctctaatcagccctctggcccaggcagcccatgttgtagcaaaccctc
aagctgaggggcagctccagtggctgaaccgccgggccaatgccctcctggccaatggcgtggagctgagagataaccagctggtggt
gccatcagagggcctgtacctcatctactcccaggtcctcttcaagggccaaggctgcccctccacccatgtgctcctcacccacaccatca
gccgcatcgccgtctcctaccagaccaaggtcaacctcctctctgccatcaagagcccctgccagagggagaccccagagggggctgag
gccaagccctggtatgagcccatctatctgggaggggtcttccagctggagaagggtgaccgactcagcgctgagatcaatcggcccgac
tatctcgactttgccgagtctgggcaggtctactttgggatcattgccctgcg
(SEQ ID NO: 38)
MSTEMHPGRGSWHEEALPKKTGGPQGSRRCLFLSLFSFLIVAGATTLFCLLHFGV
IGPQREEFPRDLSLISPLAQAVRSSSRTPSDKPVAHVVANPQAEGQLQWLNRRANALLA
NGVELRDNQLVVPSEGLYLIYSQVLFKGQGCPSTHVLLTHTISRIAVSYQTKVNLLSAIKS
PCQRETPEGAEAKPWYEPIYLGGVFQLEKGDRLSAEINRPDYLDFAESGQVYFGIIAL

One example of a TNF-alpha mutant having a mutation at M-71K in the intracytoplasmic sequence and another mutation at Y87H (mutated sequences underlined) for nucleic acid and amino acid, respectively, is as follows:

(SEQ ID NO: 39)
atgagcactgaaagcaAgatccgggacgtggagctggccgaggaggcgctccccaagaagacaggggggccccagggct
ccaggcggtgcttgttcctcagcctcttctccttcctgatcgtggcaggcgccaccacgctcttctgcctgctgcactttggagtgatcggccc
ccagagggaagagttccccagggacctctctctaatcagccctctggcccaggcagcccatgttgtagcaaaccctcaagctgaggggca
gctccagtggctgaaccgccgggccaatgccctcctggccaatggcgtggagctgagagataaccagctggtggtgccatcagagggcc
tgtacctcatctactcccaggtcctcttcaagggccaaggctgcccctccacccatgtgctcctcacccacaccatcagccgcatcgccgtct
ccCaccagaccaaggtcaacctcctctctgccatcaagagcccctgccagagggagaccccagagggggctgaggccaagccctggta
tgagcccatctatctgggaggggtcttccagctggagaagggtgaccgactcagcgctgagatcaatcggcccgactatctcgactttgcc
gagtctgggcaggtctactttgggatcattgccctgtcg
(SEQ ID NO: 40)
MSTESKIRDVELAEEALPKKTGGPQGSRRCLFLSLFSFLIVAGATTLFCLLHFGVI
GPQREEFPRDLSLISPLAQAAHVVANPQAEGQLQWLNRRANALLANGVELRDNQLVVP
SEGLYLIYSQVLFKGQGCPSTHVLLTHTISRIAVSHQTKVNLLSAIKSPCQRETPEGAEAK
PWYEPIYLGGVFQLEKGDRLSAEINRPDYLDFAESGQVYFGIIAL

One example of a TNF-alpha mutant having a mutation at S95F and C-28F (mutated sequences underlined) for nucleic acid and amino acid, respectively, is as follows:

(SEQ ID NO: 41)
atgagcactgaaagcatgatccgggacgtggagctggccgaggaggcgctccccaagaagacaggggggccccagggctc
caggcggtgcttgttcctcagcctcttctccttcctgatcgtggcaggcgccaccacgctcttctTcctgctgcactttggagtgatcggcccc
cagagggaagagttccccagggacctctctctaatcagccctctggcccaggcagcccatgttgtagcaaaccctcaagctgaggggcag
ctccagtggctgaaccgccgggccaatgccctcctggccaatggcgtggagctgagagataaccagctggtggtgccatcagagggcct
gtacctcatctactcccaggtcctcttcaagggccaaggctgcccctccacccatgtgctcctcacccacaccatcagccgcatcgccgtctc
ctaccagaccaaggtcaacctcctctTCgccatcaagagcccctgccagagggagaccccagagggggctgaggccaagccctggtat
gagcccatctatctgggaggggtcttccagctggagaagggtgaccgactcagcgctgagatcaatcggcccgactatctcgactttgccg
agtctgggcaggtctactttgggatcattgccctgtcg
(SEQ ID NO: 42)
MSTESMIRDVELAEEALPKKTGGPQGSRRCLFLSLFSFLIVAGATTLFFLLHFGVI
GPQREEFPRDLSLISPLAQAAHVVANPQAEGQLQWLNRRANALLANGVELRDNQLVVP
SEGLYLIYSQVLFKGQGCPSTHVLLTHTISRIAVSYQTKVNLLFAIKSPCQRETPEGAEAK
PWYEPIYLGGVFQLEKGDRLSAEINRPDYLDFAESGQVYFGIIAL

One example of a TNF-alpha mutant having a mutation at S1331 and S147Y (mutated sequences underlined) for nucleic acid and amino acid, respectively, is as follows:

(SEQ ID NO: 43)
atgagcactgaaagcatgatccgggacgtggagctggccgaggaggcgctccccaagaagacaggggggccccagggctc
caggcggtgcttgttcctcagcctcttctccttcctgatcgtggcaggcgccaccacgctcttctgcctgctgcactttggagtgatcggcccc
cagagggaagagttccccagggacctctctctaatcagccctctggcccaggcagcccatgttgtagcaaaccctcaagctgaggggcag
ctccagtggctgaaccgccgggccaatgccctcctggccaatggcgtggagctgagagataaccagctggtggtgccatcagagggcct
gtacctcatctactcccaggtcctcttcaagggccaaggctgcccctccacccatgtgctcctcacccacaccatcagccgcatcgccgtctc
ctaccagaccaaggtcaacctcctctctgccatcaagagcccctgccagagggagaccccagagggggctgaggccaagccctggtatg
agcccatctatctgggaggggtcttccagctggagaagggtgaccgactcaTcgctgagatcaatcggcccgactatctcgactttgccga
gtAtgggcaggtctactttgggatcattgccctgtcg
(SEQ ID NO: 44)
MSTESMIRDVELAEEALPKKTGGPQGSRRCLFLSLFSFLIVAGATTLFCLLHFGVI
GPQREEFPRDLSLISPLAQAAHVVANPQAEGQLQWLNRRANALLANGVELRDNQLVVP
SEGLYLIYSQVLFKGQGCPSTHVLLTHTISRIAVSYQTKVNLLSAIKSPCQRETPEGAEAK
PWYEPIYLGGVFQLEKGDRLIAEINRPDYLDFAEYGQVYFGIIAL

One example of a TNF-alpha mutant having a mutation at Asp143Tyr and a deletion of Ala at position −1 (mutated sequence underlined and deleted sequence shown by strikethrough) for nucleic acid and amino acid, respectively, is as follows:

(SEQ ID NO: 45)
atgagcactgaaagcatgatccgggacgtggagctggccgaggaggcgctccccaagaagacaggggggccccagggctc
caggcggtgcttgttcctcagcctcttctccttcctgatcgtggcaggcgccaccacgctcttctgcctgctgcactttggagtgatcggcccc
cagagggaagagttccccagggacctctctctaatcagccctctggcccaggcagcccatgttgtagcaaaccctcaagctgaggggcag
ctccagtggctgaaccgccgggccaatgccctcctggccaatggcgtggagctgagagataaccagctggtggtgccatcagagggcct
gtacctcatctactcccaggtcctcttcaagggccaaggctgcccctccacccatgtgctcctcacccacaccatcagccgcatcgccgtctc
ctaccagaccaaggtcaacctcctctctgccatcaagagcccctgccagagggagaccccagagggggctgaggccaagccctggtatg
agcccatctatctgggaggggtcttccagctggagaagggtgaccgactcagcgctgagatcaatcggcccgactatctcTactttgccga
gtctgggcaggtctactttgggatcattgccctgtcg
(SEQ ID NO: 46)
MSTESMIRDVELAEEALPKKTGGPQGSRRCLFLSLFSFLIVAGATTLFCLLHFGVI
GPQREEFPRDLSLISPLAQAAHVVANPQAEGQLQWLNRRANALLANGVELRDNQLVVP
SEGLYLIYSQVLFKGQGCPSTHVLLTHTISRIAVSYQTKVNLLSAIKSPCQRETPEGAEAK
PWYEPIYLGGVFQLEKGDRLSAEINRPDYLYFAESGQVYFGIIAL

Versions of SEQ ID NO:45 and SEQ ID NO:46 that lack the deleted sequences are as follows, respectively (with the mutated sequence still underlined).

(SEQ ID NO: 47)
atgagcactgaaagcatgatccgggacgtggagctggccgaggaggcgctccccaagaagacaggggggccccagggctc
caggcggtgcttgttcctcagcctcttctccttcctgatcgtggcaggcgccaccacgctcttctgcctgctgcactttggagtgatcggcccc
cagagggaagagttccccagggacctctctctaatcagccctctgcaggcagcccatgttgtagcaaaccctcaagctgaggggcagctc
cagtggctgaaccgccgggccaatgccctcctggccaatggcgtggagctgagagataaccagctggtggtgccatcagagggcctgta
cctcatctactcccaggtcctcttcaagggccaaggctgcccctccacccatgtgctcctcacccacaccatcagccgcatcgccgtctccta
ccagaccaaggtcaacctcctctctgccatcaagagcccctgccagagggagaccccagagggggctgaggccaagccctggtatgag
cccatctatctgggaggggtcttccagctggagaagggtgaccgactcagcgctgagatcaatcggcccgactatctcTactttgccgagt
ctgggcaggtctactttgggatcattgccctgtcg
(SEQ ID NO: 48)
MSTESMIRDVELAEEALPKKTGGPQGSRRCLFLSLFSFLIVAGATTLFCLLHFGVI
GPQREEFPRDLSLISPLAQAHVVANPQAEGQLQWLNRRANALLANGVELRDNQLVVPS
EGLYLIYSQVLFKGQGCPSTHVLLTHTISRIAVSYQTKVNLLSAIKSPCQRETPEGAEAKP
WYEPIYLGGVFQLEKGDRLSAEINRPDYLYFAESGQVYFGIIAL

One example of a TNF-alpha mutant having a combination of the CIK motif mutation and the above-referenced mutations are as follows, with the mutations underlined:

(SEQ ID NO: 49)
ATGCTCGAGtcgagatgagcactgaaaTGCATCCCGGAAGGGGGTCCTGGCACgaggaggc
gctccccaagaagacaggggggccccagggctccaggcggtgcttgttcctcagcctcttctccttcctgatcgtggcaggcgccaccac
gctcttctTcctgctgcactttggagtgatcggcccccagagggaagagttccccagggacctctctctaatcagccctctggcagcccat
gttgtagcaaaccctcaagctgaggggcagctccagtggctgaaccgccgggccaatgccctcctggccaatggcgtggagctgagagat
aaccagctggtggtgccatcagagggcctgtacctcatctactcccaggtcctcttcaagggccaaggctgcccctccacccatgtgctcc
tcacccacaccatcagccgcatcgccgtctccCaccagaccaaggtcaacctcctctTCgccatcaagagcccctgccagagggagacc
ccagagggggctgaggccaagccctggtatgagcccatctatctgggaggggtcttccagctggagaagggtgaccgactcaTcgctga
gatcaatcggcccgactatctcTactttgccgagtAtgggcaggtctactttgggatcattgccctgtcg
(SEQ ID NO: 50)
MSTEMHPGRGSWHEEALPKKTGGPOGSRRCLFLSLFSFLIVAGATTLFFLLHFGV
IGPQREEFPRDLSLISPLAQAHVVANPQAEGQLQWLNRRANALLANGVELRDNQLVVPS
EGLYLIYSQVLFKGQGCPSTHVLLTHTISRIAVSHQTKVNLLFAIKSPCQRETPEGAEAKP
WYEPIYLGGVFQLEKGDRLIAEINRPDYLYFAEYGQVYFGIIAL

One example of a TNFalpha mutant having a CKI mutation (5aamut) and delAla-1 to Val13 (14aa del) is as follows:

delAla-1 to Val13 (14 aa del) CKI mut 5 aa mut
(SEQ ID NO: 149)
MSTEMHPGRGSWHEEALPKKTGGPQGSRRCLFLSLFSFLI
VAGATTLFFLLHFGVIGPQREEFPRDLSLISPLQAAHVVANPQA
EGQLQWLNRRANALLANGVELRDNQLVVPSEGLYLIYSQVLF
KGQGCPSTHVLLTHTISRIAVSHQTKVNLLFAIKSPCQRETPEG
AEAKPWYEPIYLGGVFQLEKGDRLIAEINRPDYLYFAEYGQVY
FGIIALS
(SEQ ID NO: 150)
ATGAGCACTGAAATGCATCCCGGAAGGGGGTCCTGGCACGAGGAGGCGC
TCCCCAAGAAGACAGGGGGGCCCCAGGGCTCCAGGCGGTGCTTGTTCCT
CAGCCTCTTCTCCTTCCTGATCGTGGCAGGCGCCACCACGCTCTTCTTC
CTGCTGCACTTTGGAGTGATCGGCCCCCAGAGGGAAGAGTTCCCCAGGG
ACCTCTCTCTAATCAGCCCTCTGCAGGCAGCCCATGTTGTAGCAAACCC
TCAAGCTGAGGGGCAGCTCCAGTGGCTGAACCGCCGGGCCAATGCCCTC
CTGGCCAATGGCGTGGAGCTGAGAGATAACCAGCTGGTTGTGCCATCAG
AGGGCCTGTACCTCATCTACTCCCAGGTCCTCTTCAAGGGCCAAGGCTG
CCCCTCCACCCATGTGCTCCTCACCCACACCATCAGCCGCATCGCCGTC
TCCCACCAGACCAAGGTCAACCTCCTCTTCGCCATCAAGAGCCCCTGCC
AGAGGGAGACCCCAGAGGGGGCTGAGGCTAAGCCCTGGTATGAGCCCAT
CTATCTGGGAGGGGTCTTCCAGCTGGAGAAGGGTGACCGACTCATCGCT
GAGATCAATCGGCCCGACTATCTCTACTTTGCCGAGTATGGGCAGGTCT
ACTTTGGGATCATTGCCCTGTCGT

In particular embodiments, upon delivering an effective amount of one or more agents to bind to the TNF-alpha mutant-expressing BCMA CAR-targeting cells, the majority of TNF-alpha mutant-expressing cells are eliminated. In specific embodiments, greater than 50%, 55%, 60%, 65%, 70%, 75%, 80%, 85%, 90%, 95%, 96%, 97%, 98%, or 99% of cells expressing the TNF-alpha mutants are eliminated in an individual. Following recognition of a need to eliminate the cells, the delivery of the agent(s) to the individual may continue until one or more symptoms are no longer present or until a sufficient number of cells have been eliminated. The cell numbers in the individual may be monitored using the TNF-alpha mutants as markers.

Embodiments of methods of the disclosure may comprise a first step of providing an effective amount of the NK cell therapy to an individual in need thereof, wherein the cells comprise one or more nonsecretable TNF-alpha mutants; and, a second step of eliminating the cells using the TNF-alpha mutant(s) as suicide genes (directly or indirectly through cell death by any mechanism). The second step may be instigated upon onset of at least one adverse event for the individual, and that adverse event may be recognized by any means, including upon routine monitoring that may or may not be continuous from the beginning of the cell therapy. The adverse event(s) may be detected upon examination and/or testing. In cases wherein the individual has cytokine release syndrome (which may also be referred to as cytokine storm), the individual may have elevated inflammatory cytokine(s) (merely as examples: interferon-gamma, granulocyte macrophage colony-stimulating factor, IL-10, IL-6 and TNF-alpha); fever; fatigue; hypotension; hypoxia, tachycardia; nausea; capillary leak; cardiac/renal/hepatic dysfunction; or a combination thereof, for example. In cases wherein the individual has neurotoxicity, the individual may have confusion, delirium, aplasia, and/or seizures. In some cases, the individual is tested for a marker associated with onset and/or severity of cytokine release syndrome, such as C-reactive protein, IL-6, TNF-alpha, and/or ferritin

In additional embodiments, administration of one or more agents that bind the nonsecretable TNF-α during cytokine release syndrome or neurotoxicity, for example, have the added benefit of neutralizing the high levels of soluble TNF-alpha that contribute to the toxicity of the therapy. Soluble TNF-alpha is released at high levels during cytokine release syndrome and is a mediator of toxicity with CAR T-cell therapies. In such cases, the administration of TNF-alpha antibodies encompassed herein have a dual beneficial effect—i.e. selective deletion of the TNF-alpha mutant-expressing cells as well as neutralizing soluble TNF-alpha causing toxicity. Thus, embodiments of the disclosure encompass methods of eliminating or reducing the severity of cytokine release syndrome in an individual receiving, or who has received, adoptive cell therapy in which the cells express a nonsecretable TNF-alpha mutant, comprising the step of providing an effective amount of an agent that binds the nonsecretable TNF-alpha mutant, said agent causing in the individual (a) elimination of at least some of the cells of the cell therapy; and (b) reduction in levels of soluble TNF-alpha.

Embodiments of the disclosure include methods of reducing the effects of cytokine release syndrome in an individual that has received or who is receiving cell therapy with cells that express a nonsecretable TNF-alpha mutant, comprising the step of providing an effective amount of one or more agents that bind the mutant to cause in the individual (a) elimination of at least some of the cells of the cell therapy; and (b) reduction in the level of soluble TNF-alpha.

When the need arises for the TNF-alpha suicide gene to be utilized, the individual is provided an effective amount of one or more inhibitors that are able to inhibit, such as by binding directly, the TNF-alpha mutant on the surface of the cells. The inhibitor(s) may be provided to the individual systemically and/or locally in some embodiments. The inhibitor may be a polypeptide (such as an antibody), a nucleic acid, a small molecule (for example, a xanthine derivative), a peptide, or a combination thereof. In specific embodiments, the antibodies are FDA-approved. When the inhibitor is an antibody, the inhibitor may be a monoclonal antibody in at least some cases. When mixtures of antibodies are employed, one or more antibodies in the mixture may be a monoclonal antibody. Examples of small molecule TNF-alpha inhibitors include small molecules such as are described in U.S. Pat. No. 5,118,500, which is incorporated by reference herein in its entirety. Examples of polypeptide TNF-alpha inhibitors include polypeptides, such as those described in U.S. Pat. No. 6,143,866, which is incorporated by reference herein in its entirety.

In particular embodiments, at least one antibody is utilized to target the TNF-alpha mutant to trigger its activity as a suicide gene. Examples of antibodies includes at least Adalimumab, Adalimumab-atto, Certolizumab pegol, Etanercept, Etanercept-szzs, Golimumab, Infliximab, Infliximab-dyyb, or a mixture thereof, for example.

Embodiments of the disclosure include methods of reducing the risk of toxicity of a cell therapy for an individual by modifying cells of a cell therapy to express a nonsecretable TNF-alpha mutant. The cell therapy is for cancer, in specific embodiments, and it may comprise an engineered receptor that targets an antigen, including a cancer antigen.

In particular embodiments, in addition to the inventive NK cell therapy of the disclosure, the individual may have been provided, may be provided, and/or may will be provided an additional therapy for the medical condition. In cases wherein the medical condition is cancer, the individual may be provided one or more of surgery, radiation, immunotherapy (other than the cell therapy of the present disclosure), hormone therapy, gene therapy, chemotherapy, and so forth.

### III. CYTOKINES

One or more cytokines may be co-expressed from the vector as a separate polypeptide from the antigen receptor. Interleukin-15 (IL-15), for example, is tissue restricted and only under pathologic conditions is it observed at any level in the serum, or systemically. IL-15 possesses several attributes that are desirable for adoptive therapy. IL-15 is a homeostatic cytokine that induces development and cell proliferation of natural killer cells, promotes the eradication of established tumors via alleviating functional suppression of tumor-resident cells, and inhibits activation-induced cell death (AICD). In addition to IL-15, other cytokines are envisioned. These include, but are not limited to, cytokines, chemokines, and other molecules that contribute to the activation and proliferation of cells used for human application. NK cells expressing IL-15 are capable of continued supportive cytokine signaling, which is useful for their survival post-infusion.

In specific embodiments, NK cells expresses one or more exogenously provided cytokines. As one example, the cytokine is IL-15, IL-12, IL-2, IL-18, IL-21 or a combination thereof. The cytokine may be exogenously provided to the NK cells because it is expressed from an expression vector within the cell. In an alternative case, an endogenous cytokine in the cell is upregulated upon manipulation of regulation of expression of the endogenous cytokine, such as genetic recombination at the promoter site(s) of the cytokine. In cases wherein the cytokine is provided on an expression construct to the cell, the cytokine may be encoded from the same vector as the TNF-alpha mutant gene. The cytokine may be expressed as a separate polypeptide molecule as the TNF-alpha mutant and as a separate polypeptide from an engineered receptor of the cell. In some embodiments, the present disclosure concerns co-utilization of CAR and/or TCR vectors with IL-15.

### IV. VECTORS

The BCMA-targeting CARs may be delivered to the recipient NK cell by any suitable vector, including by a viral vector or by a non-viral vector. Examples of viral vectors include at least retroviral, lentiviral, adenoviral, or adeno-associated viral vectors. Examples of non-viral vectors include at least plasmids, transposons, lipids, nanoparticles, and so forth.

In cases wherein the NK cell is transduced with a vector encoding the BCMA-targeting CAR and also requires transduction of another gene or genes into the cell, such as a suicide gene and/or cytokine and/or an optional therapeutic gene product, the BCMA-targeting CAR, suicide gene, cytokine, and optional therapeutic gene may or may not be comprised on or with the same vector. In some cases, the BCMA-targeting CAR, suicide gene, cytokine, and optional therapeutic gene are expressed from the same vector molecule, such as the same viral vector molecule. In such cases, the expression of the BCMA-targeting CAR, suicide gene, cytokine, and optional therapeutic gene may or may not be regulated by the same regulatory element(s). When the BCMA-targeting CAR, suicide gene, cytokine, and optional therapeutic gene are on the same vector, they may or may not be expressed as separate polypeptides. In cases wherein they are expressed as separate polypeptides, they may be separated on the vector by a 2A element or IRES element (or both kinds may be used on the same vector once or more than once), for example.

### A. General Embodiments

One of skill in the art would be well-equipped to construct a vector through standard recombinant techniques (see, for example, Sambrook et al., 2001 and Ausubel et al., 1996, both incorporated herein by reference) for the expression of the antigen receptors of the present disclosure.

### 1. Regulatory Elements

Expression cassettes included in vectors useful in the present disclosure in particular contain (in a 5′-to-3′ direction) a eukaryotic transcriptional promoter operably linked to a protein-coding sequence, splice signals including intervening sequences, and a transcriptional termination/polyadenylation sequence. The promoters and enhancers that control the transcription of protein encoding genes in eukaryotic cells may be comprised of multiple genetic elements. The cellular machinery is able to gather and integrate the regulatory information conveyed by each element, allowing different genes to evolve distinct, often complex patterns of transcriptional regulation. A promoter used in the context of the present disclosure includes constitutive, inducible, and tissue-specific promoters, for example. In cases wherein the vector is utilized for the generation of cancer therapy, a promoter may be effective under conditions of hypoxia.

### 2. Promoter/Enhancers

The expression constructs provided herein comprise a promoter to drive expression of the antigen receptor and other cistron gene products. A promoter generally comprises a sequence that functions to position the start site for RNA synthesis. The best known example of this is the TATA box, but in some promoters lacking a TATA box, such as, for example, the promoter for the mammalian terminal deoxynucleotidyl transferase gene and the promoter for the SV40 late genes, a discrete element overlying the start site itself helps to fix the place of initiation. Additional promoter elements regulate the frequency of transcriptional initiation. Typically, these are located in the region upstream of the start site, although a number of promoters have been shown to contain functional elements downstream of the start site as well. To bring a coding sequence “under the control of” a promoter, one positions the 5′ end of the transcription initiation site of the transcriptional reading frame “downstream” of (i.e., 3′ of) the chosen promoter. The “upstream” promoter stimulates transcription of the DNA and promotes expression of the encoded RNA.

The spacing between promoter elements frequently is flexible, so that promoter function is preserved when elements are inverted or moved relative to one another. In the tk promoter, for example, the spacing between promoter elements can be increased to 50 bp apart before activity begins to decline. Depending on the promoter, it appears that individual elements can function either cooperatively or independently to activate transcription. A promoter may or may not be used in conjunction with an “enhancer,” which refers to a cis-acting regulatory sequence involved in the transcriptional activation of a nucleic acid sequence.

A promoter may be one naturally associated with a nucleic acid sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other virus, or prokaryotic or eukaryotic cell, and promoters or enhancers not “naturally occurring,” i.e., containing different elements of different transcriptional regulatory regions, and/or mutations that alter expression. For example, promoters that are most commonly used in recombinant DNA construction include the β-lactamase (penicillinase), lactose and tryptophan (trp-) promoter systems. In addition to producing nucleic acid sequences of promoters and enhancers synthetically, sequences may be produced using recombinant cloning and/or nucleic acid amplification technology, including PCR™, in connection with the compositions disclosed herein. Furthermore, it is contemplated that the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the organelle, cell type, tissue, organ, or organism chosen for expression. Those of skill in the art of molecular biology generally know the use of promoters, enhancers, and cell type combinations for protein expression, (see, for example Sambrook et al. 1989, incorporated herein by reference). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

Additionally, any promoter/enhancer combination (as per, for example, the Eukaryotic Promoter Data Base EPDB, through world wide web at epd.isb-sib.ch/) could also be used to drive expression. Use of a T3, T7 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

Non-limiting examples of promoters include early or late viral promoters, such as, SV40 early or late promoters, cytomegalovirus (CMV) immediate early promoters, Rous Sarcoma Virus (RSV) early promoters; eukaryotic cell promoters, such as, e.g., beta actin promoter, GADPH promoter, metallothionein promoter; and concatenated response element promoters, such as cyclic AMP response element promoters (cre), serum response element promoter (sre), phorbol ester promoter (TPA) and response element promoters (tre) near a minimal TATA box. It is also possible to use human growth hormone promoter sequences (e.g., the human growth hormone minimal promoter described at GenBank®, accession no. X05244, nucleotide 283-341) or a mouse mammary tumor promoter (available from the ATCC, Cat. No. ATCC 45007). In certain embodiments, the promoter is CMV IE, dectin-1, dectin-2, human CD11c, F4/80, SM22, RSV, SV40, Ad MLP, beta-actin, MHC class I or MHC class II promoter, however any other promoter that is useful to drive expression of the therapeutic gene is applicable to the practice of the present disclosure.

In certain aspects, methods of the disclosure also concern enhancer sequences, i.e., nucleic acid sequences that increase a promoter's activity and that have the potential to act in cis, and regardless of their orientation, even over relatively long distances (up to several kilobases away from the target promoter). However, enhancer function is not necessarily restricted to such long distances as they may also function in close proximity to a given promoter.

### 3. Initiation Signals and Linked Expression

A specific initiation signal also may be used in the expression constructs provided in the present disclosure for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In certain embodiments, the use of internal ribosome entry sites (IRES) elements are used to create multigene, or polycistronic messages. IRES elements are able to bypass the ribosome scanning model of 5′ methylated Cap dependent translation and begin translation at internal sites. IRES elements from two members of the picornavirus family (polio and encephalomyocarditis) have been described, as well an IRES from a mammalian message. IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message.

As detailed elsewhere herein, certain 2A sequence elements could be used to create linked- or co-expression of genes in the constructs provided in the present disclosure. For example, cleavage sequences could be used to co-express genes by linking open reading frames to form a single cistron. An exemplary cleavage sequence is the equine rhinitis A virus (E2A) or the F2A (Foot-and-mouth disease virus 2A) or a “2A-like” sequence (e.g., Thosea asigna virus 2A; T2A) or porcine teschovirus-1 (P2A). In specific embodiments, in a single vector the multiple 2A sequences are non-identical, although in alternative embodiments the same vector utilizes two or more of the same 2A sequences. Examples of 2A sequences are provided in US 2011/0065779 which is incorporated by reference herein in its entirety.

### 4. Origins of Replication

In order to propagate a vector in a host cell, it may contain one or more origins of replication sites (often termed “ori”), for example, a nucleic acid sequence corresponding to oriP of EBV as described above or a genetically engineered oriP with a similar or elevated function in programming, which is a specific nucleic acid sequence at which replication is initiated. Alternatively a replication origin of other extra-chromosomally replicating virus as described above or an autonomously replicating sequence (ARS) can be employed.

### 5. Selection and Screenable Markers

In some embodiments, NK cells comprising a construct of the present disclosure may be identified in vitro or in vivo by including a marker in the expression vector. Such markers would confer an identifiable change to the cell permitting easy identification of cells containing the expression vector. Generally, a selection marker is one that confers a property that allows for selection. A positive selection marker is one in which the presence of the marker allows for its selection, while a negative selection marker is one in which its presence prevents its selection. An example of a positive selection marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selection markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes as negative selection markers such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selection and screenable markers are well known to one of skill in the art.

### B. Multicistronic Vectors

In particular embodiments, the BCMA-targeting CAR, suicide gene, cytokine, and/or optional therapeutic gene are expressed from a multicistronic vector (The term “cistron” as used herein refers to a nucleic acid sequence from which a gene product may be produced). In specific embodiments, the multicistronic vector encodes the BCMA-targeting CAR, the TNF-alpha mutant and at least one cytokine, and/or engineered receptor, such as a T-cell receptor and/or an additional non-BCMA-targeting CAR. In some cases, the multicistronic vector encodes at least one BCMA-targeting CAR, at least one TNF-alpha mutant, and at least one cytokine. The cytokine may be of a particular type of cytokine, such as human or mouse or any species. In specific cases, the cytokine is IL15, IL12, IL2, IL18, and/or IL21.

In certain embodiments, the present disclosure provides a flexible, modular system (the term “modular” as used herein refers to a cistron or component of a cistron that allows for interchangeability thereof, such as by removal and replacement of an entire cistron or of a component of a cistron, respectively, for example by using standard recombination techniques) utilizing a polycistronic vector having the ability to express multiple cistrons at substantially identical levels. The system may be used for cell engineering allowing for combinatorial expression (including overexpression) of multiple genes. In specific embodiments, one or more of the genes expressed by the vector includes one, two, or more antigen receptors. The multiple genes may comprise, but are not limited to, CARs, TCRs, cytokines, chemokines, homing receptors, CRISPR/Cas9-mediated gene mutations, decoy receptors, cytokine receptors, chimeric cytokine receptors, and so forth. The vector may further comprise: (1) one or more reporters, for example fluorescent or enzymatic reporters, such as for cellular assays and animal imaging; (2) one or more cytokines or other signaling molecules; and/or (3) a suicide gene.

In specific cases, the vector may comprise at least 4 cistrons separated by cleavage sites of any kind, such as 2A cleavage sites. The vector may or may not be Moloney Murine Leukemia Virus (MoMLV or MMLV)-based including the 3′ and 5′ LTR with the psi packaging sequence in a pUC19 backbone. The vector may comprise 4 or more cistrons with three or more 2A cleavage sites and multiple ORFs for gene swapping. The system allows for combinatorial overexpression of multiple genes (7 or more) that are flanked by restriction site(s) for rapid integration through subcloning, and the system also includes at least three 2A self-cleavage sites, in some embodiments. Thus, the system allows for expression of multiple CARs, TCRs, signaling molecules, cytokines, cytokine receptors, and/or homing receptors. This system may also be applied to other viral and non-viral vectors, including but not limited lentivirus, adenovirus AAV, as well as non-viral plasmids.

The modular nature of the system also enables efficient subcloning of a gene into each of the 4 cistrons in the polycistronic expression vector and the swapping of genes, such as for rapid testing. Restriction sites strategically located in the polycistronic expression vector allow for swapping of genes with efficiency.

Embodiments of the disclosure encompass systems that utilize a polycistronic vector wherein at least part of the vector is modular, for example by allowing removal and replacement of one or more cistrons (or component(s) of one or more cistrons), such as by utilizing one or more restriction enzyme sites whose identity and location are specifically selected to facilitate the modular use of the vector. The vector also has embodiments wherein multiple of the cistrons are translated into a single polypeptide and processed into separate polypeptides, thereby imparting an advantage for the vector to express separate gene products in substantially equimolar concentrations.

The vector of the disclosure is configured for modularity to be able to change one or more cistrons of the vector and/or to change one or more components of one or more particular cistrons. The vector may be designed to utilize unique restriction enzyme sites flanking the ends of one or more cistrons and/or flanking the ends of one or more components of a particular cistron.

Embodiments of the disclosure include polycistronic vectors comprising at least two, at least three, or at least four cistrons each flanked by one or more restriction enzyme sites, wherein at least one cistron encodes for at least one antigen receptor. In some cases, two, three, four, or more of the cistrons are translated into a single polypeptide and cleaved into separate polypeptides, whereas in other cases multiple of the cistrons are translated into a single polypeptide and cleaved into separate polypeptides. Adjacent cistrons on the vector may be separated by a self cleavage site, such as a 2A self cleavage site. In some cases each of the cistrons express separate polypeptides from the vector. On particular cases, adjacent cistrons on the vector are separated by an IRES element.

In certain embodiments, the present disclosure provides a system for cell engineering allowing for combinatorial expression, including overexpression, of multiple cistrons that may include one, two, or more antigen receptors, for example. In particular embodiments, the use of a polycistronic vector as described herein allows for the vector to produce equimolar levels of multiple gene products from the same mRNA. The multiple genes may comprise, but are not limited to, CARs, TCRs, cytokines, chemokines, homing receptors, CRISPR/Cas9-mediated gene mutations, decoy receptors, cytokine receptors, chimeric cytokine receptors, and so forth. The vector may further comprise one or more fluorescent or enzymatic reporters, such as for cellular assays and animal imaging. The vector may also comprise a suicide gene product for termination of cells harboring the vector when they are no longer needed or become deleterious to a host to which they have been provided.

In particular embodiments of the disclosure, at least one of the cistrons on the vector comprises two or more modular components, wherein each of the modular components within a cistron is flanked by one or more restriction enzyme sites. A cistron may comprise three, four, or five modular components, for example. In at least some cases, a cistron encodes an antigen receptor having different parts of the receptor encoded by corresponding modular components. A first modular component of a cistron may encode an antigen binding domain of the receptor. In addition, a second modular component of a cistron may encode a hinge region of the receptor. In addition, a third modular component of a cistron may encode a transmembrane domain of the receptor. In addition, a fourth modular component of a cistron may encode a first costimulatory domain. In addition, a fifth modular component of a cistron may encode a second costimulatory domain. In addition, a sixth modular component of a cistron may encode a signaling domain.

In particular aspects of the disclosure, two different cistrons on the vector each encode non-identical antigen receptors. Both antigen receptors may be encoded by a cistron comprising two or more modular components, including separate cistrons comprising two or more modular components. The antigen receptor may be a chimeric antigen receptor (CAR) and/or T cell receptor (TCR), for example.

In specific embodiments, the vector is a viral vector (retroviral vector, lentiviral vector, adenoviral vector, or adeno-associated viral vector, for example) or a non-viral vector. The vector may comprise a Moloney Murine Leukemia Virus (MMLV) 5′ LTR, 3′ LTR, and/or psi packaging element. In specific cases, the psi packaging is incorporated between the 5′ LTR and the antigen receptor coding sequence. The vector may or may not comprise pUC19 sequence. In some aspects of the vector, at least one cistron encodes for a cytokine (interleukin 15 (IL-15), IL-7, IL-21, or IL-2, for example), chemokine, cytokine receptor, and/or homing receptor.

When 2A cleavages sites are utilized in the vector, the 2A cleavage site may comprise a P2A, T2A, E2A and/or F2A site.

In addition to one cistron encoding a BCMA-targeting CAR, any cistron of the vector may comprise a suicide gene. Any cistron of the vector may encode a reporter gene. In specific embodiments, a first cistron encodes a suicide gene, a second cistron encodes a BCMA-targeting CAR, a third cistron encodes a reporter gene, and a fourth cistron encodes a cytokine. In certain embodiments, a first cistron encodes a suicide gene, a second cistron encodes a a BCMA-targeting CAR, a third cistron encodes a second CAR or another antigen receptor, and a fourth cistron encodes a cytokine. In specific embodiments, different parts of the a BCMA-targeting CAR and/or another receptor are encoded by corresponding modular components and a first component of the second cistron encodes an antigen binding domain, a second component encodes a hinge and/or transmembrane domain, a third component encodes a costimulatory domain, and a fourth component encodes a signaling domain.

The methods and compositions of the disclosure encompass any suitable order of cistrons on a single vector.

In particular embodiments, multiple cistrons of the vector are separated by one or more elements that provide for expression of genes from the corresponding multiple cistrons into a single transcript. The single transcript is subsequently translated to produce a multi-protein polypeptide that is processed (for example, by cleavage) such that the proteins become separate protein molecules. An exemplary element is a site that encodes a self-cleaving peptide, such as a 2A peptide cleavage sequence. Other cleavage sites include furin cleavage site or a Tobacco Etch Virus (TEV) cleavage site. In other cases, the cistrons of the vector are separated by one or more elements that provide for distinct translation of the separate cistrons (such as IRES sequences). In some cases, the vector utilizes a combination of both types of elements.

The genetic cargo of interest may include 1, 2, 3, 4, 5, 6, 7, 8, 9, 10, or more cistrons comprising at least one ORF that may be expressed from the vector. Embodiments of the disclosure include the vector in states wherein the genetic cargo of interest may not be presently housed in the vector but the vector still retains one or more structural or housekeeping elements required for expression and/or further processing of cistrons when they are present (such as promoter(s), multiple 2A sequences, etc.). The vector may have multiple cistrons that are able to be translated into a single polypeptide and processed into separate polypeptides (such as by using 2A self cleavage sites between adjacent cistrons). In alternative embodiments, multiple of the cistrons are expressed as separate polypeptides (such as by using IRES elements between adjacent cistrons).

In specific cases, the structure of the genetic cargo of interest in the vector may be as follows:

Cistron 1-2A-Cistron 2-2A-Cistron 3-2A-Cistron 4,

wherein in specific embodiments the cistron 1, cistron 2, cistron 3, and cistron 4 are different genes. In at least some cases, the 2A sequences within a vector may or may not be identical.

In specific embodiments, at least one of the cistrons encodes a suicide gene. In some embodiments, at least one of the cistrons encodes a cytokine. In certain embodiments, at least one cistron encodes a BCMA-targeting CAR. A cistron may or may not encode a reporter gene. In certain embodiments, at least two cistrons encode two different antigen receptors (e.g., CARs and/or TCRs). A cistron may or may not encode a reporter gene.

In particular configurations of the genetic cargo of interest, a single vector may comprise a cistron that encodes a BCMA-targeting CAR and a cistron that encodes a second antigen receptor that is non-identical to the BCMA-targeting CAR eptor. In specific embodiments, the first antigen receptor encodes a a BCMA-targeting CAR and the second antigen receptor encodes a TCR, or vice versa. In particular embodiments, a vector comprising separate cistrons that respectively encode a BCMA-targeting CAR and a second antigen receptor also comprises a third cistron that encodes a cytokine or chemokine and a fourth cistron that encodes a suicide gene. However, the suicide gene and/or the cytokine (or chemokine) may not be present on the vector.

In particular embodiments, at least one cistron comprises multiple component(s) themselves that are modular. For example, one cistron may encode a multi-component gene product, such as an antigen receptor having multiple parts; in specific cases the antigen receptor is encoded from a single cistron, thereby ultimately producing a single polypeptide. The cistron encoding multiple components may have the multiple components separated by 1, 2, 3, 4, 5, or more restriction enzyme digestion sites, including 1, 2, 3, 4, 5, or more restriction enzyme digestion sites that are unique to the vector comprising the cistron (FIGS. 1A and 1B). In specific embodiments, a cistron having multiple components encodes an antigen receptor having multiple corresponding parts each attributing a unique function to the receptor. In a specific embodiment, each or the majority of components of the multi-component cistrons is separated by one or more restriction enzyme digestion sites that are unique to the vector, allowing the interchangeability of separate components when desired.

As an illustration, the modularity of one example of a multi-component cistron is configured as follows, wherein there are one or more unique restriction enzyme sites as represented by each X:

component 1—X₁-component 2—X₂-component 3—X₃-component 4—X₄-component 5—X₅- etc.

In specific embodiments, each component of a multi-component cistron corresponds to a different part of an encoded antigen receptor, such as a BCMA-targeting CAR. In illustrative embodiments, component 1 may encode a BCMA antigen-binding domain of the receptor; component 2 may encode a hinge domain of the receptor; component 3 may encode a transmembrane domain of the receptor; component 4 may encode a costimulatory domain of the receptor, and component 5 may encode a signaling domain of the receptor. In specific embodiments, a BCMA-targeting CAR may comprise one or more costimulatory domains, each separated by unique restriction enzyme digestion sites for interchangeability of the costimulatory domain(s) within the receptor.

In specific embodiments, there is a polycistronic vector having four separate cistrons where adjacent cistrons are separated by a 2A cleavage site, although in specific embodiments instead of a 2A cleavage site there is an element that directly or indirectly causes separate polypeptides to be produced from the cistrons (such as an IRES sequence). For example, four separate cistrons may be separated by three 2A peptide cleavage sites, and each cistron has restriction sites (X₁, X₂, etc.) flanking each end of the cistron to allow for interchangeability of the particular cistron, such as with another cistron or other type of sequence, and upon using standard recombination techniques. In specific embodiments, the restriction enzyme site(s) that flank each of the cistrons is unique to the vector to allow ease of recombination, although in alternative embodiments the restriction enzyme site is not unique to the vector.

In particular embodiments, the vector provides for a unique, second level of modularity by allowing for interchangeability within a particular cistron, including within multiple components of a particular cistron. The multiple components of a particular cistron may be separated by one or more restriction enzyme sites, including those unique to the vector, to allow for interchangeability of one or more components within the cistron. As an example, cistron 2 may comprise five separate components, although there may be 2, 3, 4, 5, 6, or more components per cistron. As an example, a vector may include cistron 2 that has five components each separated by unique enzyme restriction sites X₉, X₁₀, X₁₁, X₁₂, X₁₃, and X₁₄, to allow for standard recombination to exchange different components 1, 2, 3, 4, and/or 5. In some cases, there may be multiple restriction enzyme sites between the different components (that are unique, although alternatively one or more are not unique) and there may be sequence in between the multiple restriction enzyme sites (although alternatively there may not be). In certain embodiments, all components encoded by a cistron are designed for the purpose of being interchangeable. In particular cases, one or more components of a cistron are designed to be interchangeable, whereas one or more other components of the cistron may not be designed to be interchangeable.

In specific embodiments, a cistron encodes a BCMA-targeting CAR molecule having multiple components. For example, cistron 2 may be comprised of sequence that encodes a BCMA-targeting CAR molecule having its separate components represented by component 1, component 2, component 3, etc. The CAR molecule may comprise 2, 3, 4, 5, 6, 7, 8, or more interchangeable components. In a specific example, component 1 encodes a BCMA scFv; component 2 encodes a hinge; component 3 encodes a transmembrane domain; component 4 encodes a costimulatory domain (although there may also be component 4′ that encodes a second or more costimulatory domain flanked by restriction sites for exchange); and component 5 encodes a signaling domain. In a particular example, component 1 encodes a BCMA scFv; component 2 encodes a IgG1 hinge and/or transmembrane domain; component 3 encodes CD28; and component 4 encodes CD3 zeta.

One of skill in the art recognizes in the design of the vector that the various cistrons and components must be configured such that they are kept in frame when necessary.

In a particular example, cistron 1 encodes a suicide gene; cistron 2 encodes a BCMA-targeting CAR; cistron 3 encodes a reporter gene; cistron 4 encodes a cytokine; component 1 of cistron 2 encodes a BCMA scFv; component 2 of cistron 2 encodes IgG1 hinge; component 3 of cistron 2 encodes CD28; and component 4 encodes CD3 zeta.

A restriction enzyme site may be of any kind and may include any number of bases in its recognition site, such as between 4 and 8 bases; the number of bases in the recognition site may be at least 4, 5, 6, 7, 8, or more. The site when cut may produce a blunt cut or sticky ends. The restriction enzyme may be of Type I, Type II, Type III, or Type IV, for example. Restriction enzyme sites may be obtained from available databases, such as Integrated relational Enzyme database (IntEnz) or BRENDA (The Comprehensive Enzyme Information System).

Exemplary vectors may be circular and by convention, where position 1 (12 o'clock position at the top of the circle, with the rest of the sequence in clock-wise direction) is set at the start of 5′ LTR.

In embodiments wherein self-cleaving 2A peptides are utilized, the 2A peptides may be 18-22 amino-acid (aa)-long viral oligopeptides that mediate “cleavage” of polypeptides during translation in eukaryotic cells. The designation “2A” refers to a specific region of the viral genome and different viral 2As have generally been named after the virus they were derived from. The first discovered 2A was F2A (foot-and-mouth disease virus), after which E2A (equine rhinitis A virus), P2A (porcine teschovirus-1 2A), and T2A (thosea asigna virus 2A) were also identified. The mechanism of 2A-mediated “self-cleavage” was discovered to be ribosome skipping the formation of a glycyl-prolyl peptide bond at the C-terminus of the 2A. A highly conserved sequence GDVEXNPGP (SEQ ID NO:51) is shared by different 2As at the C-terminus, and is useful for the generation of steric hindrance and ribosome skipping. Successful skipping and recommencement of translation results in two “cleaved” proteins. Examples of 2A sequences are as follows:

T2A:
(SEQ ID NO: 52)
(GSG)EGRGSLLTCGDVEENPGP
P2A:
(SEQ ID NO: 53)
(GSG)ATNFSLLKQAGDVEENPGP
E2A:
(SEQ ID NO: 54)
(GSG)QCTNYALLKLAGDVESNPGP
F2A:
(SEQ ID NO: 55)
(GSG)VKQTLNFDLLKLAGDVESNPGP

In specific cases, the vector may be a γ-retroviral transfer vector. The retroviral transfer vector may comprises a backbone based on a plasmid, such as the pUC19 plasmid (large fragment (2.63 kb) in between HindIII and EcoRI restriction enzyme sites). The backbone may carry viral components from Moloney Murine Leukemia Virus (MoMLV) including 5′ LTR, psi packaging sequence, and 3′ LTR. LTRs are long terminal repeats found on either side of a retroviral provirus, and in the case of a transfer vector, brackets the genetic cargo of interest, such as BCMA-targeting CARs and associated components. The psi packaging sequence, which is a target site for packaging by nucleocapsid, is also incorporated in cis, sandwiched between the 5′ LTR and the CAR coding sequence. Thus, the basic structure of an example of a transfer vector can be configured as such: pUC19 sequence—5′ LTR—psi packaging sequence—genetic cargo of interest—3′ LTR—pUC19 sequence. This system may also be applied to other viral and non-viral vectors, including but not limited lentivirus, adenovirus AAV, as well as non-viral plasmids.

### V. CELLS

The present disclosure encompasses immune cells or stem cells of any kind that harbor a vector that encodes a BCMA-targeting CAR and that also may encode at least one cytokine and at least one suicide gene. In some cases, different vectors encode the CAR vs. encodes the suicide gene and/or cytokine. The NK cells may be derived from cord blood, peripheral blood, induced pluripotent stem cells (iPSCs), hematopoietic stem cells (HSCs), or bone marrow. The NK cells may be derived from a cell line such as, but not limited to, NK-92 cells, for example. The NK cell may be a cord blood mononuclear cell, such as a CD56+NK cell.

The present disclosure encompasses immune cells of any kind, including conventional T cells, NK cells, gamma-delta T cells, NKT and invariant NK T cells, regulatory T cells, macrophages, B cells, tumor infiltrating lymphocytes, or a mixture thereof.

In some cases, the cells have been expanded in the presence of an effective amount of universal antigen presenting cells (UAPCs), including in any suitable ratio. The cells may be cultured with the UAPCs at a ratio of 10:1 to 1:10; 9:1 to 1:9; 8:1 to 1:8; 7:1 to 1:7; 6:1 to 1:6; 5:1 to 1:5; 4:1 to 1:4; 3:1 to 1:3; 2:1 to 1:2; or 1:1, including at a ratio of 1:2, for example. In some cases, the NK cells were expanded in the presence of IL-2, such as at a concentration of 10-500, 10-400, 10-300, 10-200, 10-100, 10-50, 100-500, 100-400, 100-300, 100-200, 200-500, 200-400, 200-300, 300-500, 300-400, or 400-500 U/mL.

Following genetic modification with the vector(s), the NK cells may be immediately infused or may be stored. In certain aspects, following genetic modification, the cells may be propagated for days, weeks, or months ex vivo as a bulk population within about 1, 2, 3, 4, 5 days or more following gene transfer into cells. In a further aspect, the transfectants are cloned and a clone demonstrating presence of a single integrated or episomally maintained expression cassette or plasmid, and expression of the BCMA-targeting CAR is expanded ex vivo. The clone selected for expansion demonstrates the capacity to specifically recognize and lyse BCMA-expressing target cells. The recombinant immune cells may be expanded by stimulation with IL-2, or other cytokines that bind the common gamma-chain (e.g., IL-7, IL-12, IL-15, IL-21, and others). The recombinant immune cells may be expanded by stimulation with artificial antigen presenting cells. In a further aspect, the genetically modified cells may be cryopreserved.

Embodiments of the disclosure encompass cells that express one or more BCMA-targeting CARs and one or more TNF-alpha mutants as encompassed herein. The NK cell comprises a recombinant nucleic acid that encodes one or more BCMA-targeting CARs and one or more engineered nonsecretable, membrane bound TNF-alpha mutant polypeptides, in specific embodiments. In specific embodiments, in addition to expressing one or more BCMA-targeting CARs and TNF-alpha mutant polypeptides, the cell also comprises a nucleic acid that encodes one or more therapeutic gene products.

The cells may be obtained from an individual directly or may be obtained from a depository or other storage facility. The cells as therapy may be autologous or allogeneic with respect to the individual to which the cells are provided as therapy.

The cells may be from an individual in need of therapy for a medical condition, and following their manipulation to express the BCMA-targeting CAR, optional TNF-alpha mutant and optional therapeutic gene product (using standard techniques for transduction and expansion for adoptive cell therapy, for example), they may be provided back to the individual from which they were originally sourced. In some cases, the cells are stored for later use for the individual or another individual.

The NK cells that harbor the BCMA-targeting CAR that may be needed to be eliminated by a suicide gene, such as a TNF-alpha suicide gene, may be of any kind. The cells may be comprised in a population of cells, and that population may have a majority that are transduced with one or more BCMA-targeting CARs and/or one or more TNF-alpha mutant suicide genes and/or one or more cytokines. A cell population may comprise 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% of NK cells that are transduced with one or more BCMA-targeting CARs and/or one or more TNF-alpha mutant suicide genes and/or one or more cytokines. The one or more BCMA-targeting CARs and/or one or more TNF-alpha mutant suicide genes and/or one or more cytokines may be separate polypeptides.

The NK cells may be produced with the one or more BCMA-targeting CARs and/or one or more TNF-alpha mutant suicide genes and/or one or more cytokines for the intent of being modular with respect to a specific purpose. For example, cells may be generated, including for commercial distribution, expressing a BCMA-targeting CARs and/or one or more TNF-alpha mutant suicide genes and/or one or more cytokines (or distributed with a nucleic acid that encodes the mutant for subsequent transduction), and a user may modify them to express one or more other genes of interest (including therapeutic genes) dependent upon their intended purpose(s). For instance, an individual interested in treating BCMA-positive cancer may obtain or generate TNF-alpha mutant-expressing cells and modify them to express a CAR comprising a BCMA-specific scFv, or vice versa.

In particular embodiments, the genome of the transduced NK cells expressing the one or more BCMA-targeting CARs and/or one or more TNF-alpha mutant suicide genes and/or one or more cytokines may be modified. The genome may be modified in any manner, but in specific embodiments the genome is modified by CRISPR gene editing, for example. The genome of the cells may be modified to enhance effectiveness of the cells for any purpose. Specific examples of genes that may be modified in the cells includes the following: knockout of ADAM13/TACE, increase resistance of TNF-alpha mutant expressing cells to the tumor microenvironment such as TGF-beta receptor 1 or 2, IDO, checkpoint molecules such as PD1, TIGIT, KLRG1, TIM3, etc.

### VI. METHODS OF TREATMENT

In various embodiments BCMA-targeting CAR constructs, nucleic acid sequences, vectors, host cells and so forth as contemplated herein and/or pharmaceutical compositions comprising the same are used for the prevention, treatment or amelioration of a cancerous disease, such as a tumorous disease. In particular embodiments, the pharmaceutical composition of the present disclosure may be particularly useful in preventing, ameliorating and/or treating cancer, including cancer that express BCMA and that may or may not be solid tumors, for example.

The NK cells for which the BCMA-targeting CAR is utilized may be NK, T cells, or induced NKT cells engineered for cell therapy for mammals, in particular embodiments. In such cases where the cells are NK cells, the NK cell therapy may be of any kind and the NK cells may be of any kind. In specific embodiments, the cells are NK cells that have been engineered to express one or more BCMA-targeting CARs and/or one or more TNF-alpha mutant suicide genes and/or one or more cytokines. In specific embodiments, the cells are NK cells that are transduced with a BCMA-targeting CAR.

In particular embodiments, the present disclosure contemplates, in part, BCMA CAR-expressing cells, BCMA-targeting CAR constructs, BCMA-targeting CAR nucleic acid molecules and BCMA-targeting CAR vectors that can administered either alone or in any combination using standard vectors and/or gene delivery systems, and in at least some aspects, together with a pharmaceutically acceptable carrier or excipient. In certain embodiments, subsequent to administration, the nucleic acid molecules or vectors may be stably integrated into the genome of the subject.

In specific embodiments, viral vectors may be used that are specific for certain cells or tissues and persist in NK cells. Suitable pharmaceutical carriers and excipients are well known in the art. The compositions prepared according to the disclosure can be used for the prevention or treatment or delaying the above identified diseases.

Furthermore, the disclosure relates to a method for the prevention, treatment or amelioration of a tumorous disease comprising the step of administering to a subject in the need thereof an effective amount of cells that express a BCMA-targeting CAR, a nucleic acid sequence, a vector, as contemplated herein and/or produced by a process as contemplated herein.

Possible indications for administration of the composition(s) of the exemplary BCMA-targeting CAR cells are cancerous diseases, including tumorous diseases, including B cell malignancies, multiple myeloma, breast cancer, or lung cancer, for example. Exemplary indications for administration of the composition(s) of BCMA-targeting CAR cells are cancerous diseases, including any malignancies that express BCMA. The administration of the composition(s) of the disclosure is useful for all stages and types of cancer, including for minimal residual disease, early cancer, advanced cancer, and/or metastatic cancer and/or refractory cancer, for example.

The disclosure further encompasses co-administration protocols with other compounds, e.g. bispecific antibody constructs, targeted toxins or other compounds, which act via immune cells. The clinical regimen for co-administration of the inventive compound(s) may encompass co-administration at the same time, before or after the administration of the other component. Particular combination therapies include chemotherapy, radiation, surgery, hormone therapy, or other types of immunotherapy.

Embodiments relate to a kit comprising a BCMA-targeting CAR construct as defined herein, a nucleic acid sequence as defined herein, a vector as defined herein and/or a host as defined herein. It is also contemplated that the kit of this disclosure comprises a pharmaceutical composition as described herein above, either alone or in combination with further medicaments to be administered to an individual in need of medical treatment or intervention.

A. Pharmaceutical Compositions

Also provided herein are pharmaceutical compositions and formulations comprising transduced NK cells and a pharmaceutically acceptable carrier. The transduced cells may be comprised in a media suitable for transfer to an individual and/or media suitable for preservation, such as cryopreservation, including prior to transfer to an individual.

Pharmaceutical compositions and formulations as described herein can be prepared by mixing the active ingredients (such as the cells) having the desired degree of purity with one or more optional pharmaceutically acceptable carriers (Remington's Pharmaceutical Sciences 22^{nd} edition, 2012), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

B. Combination Therapies

In certain embodiments, the compositions and methods of the present embodiments involve an immune cell population in combination with at least one additional therapy. The additional therapy may be radiation therapy, surgery (e.g., lumpectomy and a mastectomy), chemotherapy, gene therapy, DNA therapy, viral therapy, RNA therapy, immunotherapy, bone marrow transplantation, nanotherapy, monoclonal antibody therapy, hormone therapy, or a combination of the foregoing. The additional therapy may be in the form of adjuvant or neoadjuvant therapy.

In some embodiments, the additional therapy is the administration of small molecule enzymatic inhibitor or anti-metastatic agent. In some embodiments, the additional therapy is the administration of side-effect limiting agents (e.g., agents intended to lessen the occurrence and/or severity of side effects of treatment, such as anti-nausea agents, etc.). In some embodiments, the additional therapy is radiation therapy. In some embodiments, the additional therapy is surgery. In some embodiments, the additional therapy is a combination of radiation therapy and surgery. In some embodiments, the additional therapy is gamma irradiation. In some embodiments, the additional therapy is therapy targeting PBK/AKT/mTOR pathway, HSP90 inhibitor, tubulin inhibitor, apoptosis inhibitor, and/or chemopreventative agent. The additional therapy may be one or more of the chemotherapeutic agents known in the art.

An immune cell therapy may be administered before, during, after, or in various combinations relative to an additional cancer therapy, such as immune checkpoint therapy. The administrations may be in intervals ranging from concurrently to minutes to days to weeks. In embodiments where the immune cell therapy is provided to a patient separately from an additional therapeutic agent, one would generally ensure that a significant period of time did not expire between the time of each delivery, such that the two compounds would still be able to exert an advantageously combined effect on the patient. In such instances, it is contemplated that one may provide a patient with the antibody therapy and the anti-cancer therapy within about 12 to 24 or 72 h of each other and, more particularly, within about 6-12 h of each other. In some situations it may be desirable to extend the time period for treatment significantly where several days (2, 3, 4, 5, 6, or 7) to several weeks (1, 2, 3, 4, 5, 6, 7, or 8) lapse between respective administrations.

Various combinations may be employed. For the example below an immune cell therapy is “A” and an anti-cancer therapy is “B”:

A/B/A B/A/B B/B/A A/A/B A/B/B B/A/A A/B/B/B
B/A/B/B B/B/B/A B/B/A/B A/A/B/B A/B/A/B
A/B/B/A B/B/A/A B/A/B/A B/A/A/B A/A/A/B B/A/A/A
A/B/A/A A/A/B/A

Administration of any compound or cell therapy of the present embodiments to a patient will follow general protocols for the administration of such compounds, taking into account the toxicity, if any, of the agents. Therefore, in some embodiments there is a step of monitoring toxicity that is attributable to combination therapy.

1. Chemotherapy

A wide variety of chemotherapeutic agents may be used in accordance with the present embodiments. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis.

Examples of chemotherapeutic agents include alkylating agents, such as thiotepa and cyclophosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammaII and calicheamicin omegaII); dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids, such as retinoic acid; capecitabine; carboplatin, procarbazine, plicomycin, gemcitabien, navelbine, farnesyl-protein tansferase inhibitors, transplatinum, and pharmaceutically acceptable salts, acids, or derivatives of any of the above.

2. Radiotherapy

Other factors that cause DNA damage and have been used extensively include what are commonly known as γ-rays, X-rays, and/or the directed delivery of radioisotopes to tumor cells. Other forms of DNA damaging factors are also contemplated, such as microwaves, proton beam irradiation (U.S. Pat. Nos. 5,760,395 and 4,870,287), and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

3. Immunotherapy

The skilled artisan will understand that additional immunotherapies may be used in combination or in conjunction with methods of the embodiments. In the context of cancer treatment, immunotherapeutics, generally, rely on the use of immune effector cells and molecules to target and destroy cancer cells. Rituximab (RITUXAN®) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve as a targeting agent. Alternatively, the effector may be a lymphocyte carrying a surface molecule that interacts, either directly or indirectly, with a tumor cell target. Various effector cells include cytotoxic T cells and NK cells

Antibody-drug conjugates have emerged as a breakthrough approach to the development of cancer therapeutics. Cancer is one of the leading causes of deaths in the world. Antibody-drug conjugates (ADCs) comprise monoclonal antibodies (MAbs) that are covalently linked to cell-killing drugs. This approach combines the high specificity of MAbs against their antigen targets with highly potent cytotoxic drugs, resulting in “armed” MAbs that deliver the payload (drug) to tumor cells with enriched levels of the antigen. Targeted delivery of the drug also minimizes its exposure in normal tissues, resulting in decreased toxicity and improved therapeutic index. The approval of two ADC drugs, ADCETRIS® (brentuximab vedotin) in 2011 and KADCYLA® (trastuzumab emtansine or T-DM1) in 2013 by FDA validated the approach. There are currently more than 30 ADC drug candidates in various stages of clinical trials for cancer treatment (Leal et al., 2014). As antibody engineering and linker-payload optimization are becoming more and more mature, the discovery and development of new ADCs are increasingly dependent on the identification and validation of new targets that are suitable to this approach and the generation of targeting MAbs. Two criteria for ADC targets are upregulated/high levels of expression in tumor cells and robust internalization.

In one aspect of immunotherapy, the tumor cell must bear some marker that is amenable to targeting, i.e., is not present on the majority of other cells. Many tumor markers exist and any of these may be suitable for targeting in the context of the present embodiments. Common tumor markers include CD20, carcinoembryonic antigen, tyrosinase (p9′7), gp68, TAG-72, HMFG, Sialyl Lewis Antigen, MucA, MucB, PLAP, laminin receptor, erb B, and p155. An alternative aspect of immunotherapy is to combine anticancer effects with immune stimulatory effects. Immune stimulating molecules also exist including: cytokines, such as IL-2, IL-4, IL-12, GM-CSF, gamma-IFN, chemokines, such as MIP-1, MCP-1, IL-8, and growth factors, such as FLT3 ligand.

Examples of immunotherapies currently under investigation or in use are immune adjuvants, e.g., Mycobacterium bovis, Plasmodium falciparum, dinitrochlorobenzene, and aromatic compounds (U.S. Pat. Nos. 5,801,005 and 5,739,169; Hui and Hashimoto, 1998; Christodoulides et al., 1998); cytokine therapy, e.g., interferons □, □□ and □, IL-1, GM-CSF, and TNF (Bukowski et al., 1998; Davidson et al., 1998; Hellstrand et al., 1998); gene therapy, e.g., TNF, IL-1, IL-2, and p53 (Qin et al., 1998; Austin-Ward and Villaseca, 1998; U.S. Pat. Nos. 5,830,880 and 5,846,945); and monoclonal antibodies, e.g., anti-CD20, anti-ganglioside GM2, and anti-p185 (Hollander, 2012; Hanibuchi et al., 1998; U.S. Pat. No. 5,824,311). It is contemplated that one or more anti-cancer therapies may be employed with the antibody therapies described herein.

In some embodiments, the immunotherapy may be an immune checkpoint inhibitor. Immune checkpoints either turn up a signal (e.g., co-stimulatory molecules) or turn down a signal. Inhibitory immune checkpoints that may be targeted by immune checkpoint blockade include adenosine A2A receptor (A2AR), B7-H3 (also known as CD276), B and T lymphocyte attenuator (BTLA), cytotoxic T-lymphocyte-associated protein 4 (CTLA-4, also known as CD152), indoleamine 2,3-dioxygenase (IDO), killer-cell immunoglobulin (KIR), lymphocyte activation gene-3 (LAG3), programmed death 1 (PD-1), T-cell immunoglobulin domain and mucin domain 3 (TIM-3) and V-domain Ig suppressor of T cell activation (VISTA). In particular, the immune checkpoint inhibitors target the PD-1 axis and/or CTLA-4.

The immune checkpoint inhibitors may be drugs such as small molecules, recombinant forms of ligand or receptors, or, in particular, are antibodies, such as human antibodies (e.g., International Patent Publication WO2015016718; Pardoll, Nat Rev Cancer, 12(4): 252-64, 2012; both incorporated herein by reference). Known inhibitors of the immune checkpoint proteins or analogs thereof may be used, in particular chimerized, humanized or human forms of antibodies may be used. As the skilled person will know, alternative and/or equivalent names may be in use for certain antibodies mentioned in the present disclosure. Such alternative and/or equivalent names are interchangeable in the context of the present disclosure. For example it is known that lambrolizumab is also known under the alternative and equivalent names MK-3475 and pembrolizumab.

In some embodiments, the PD-1 binding antagonist is a molecule that inhibits the binding of PD-1 to its ligand binding partners. In a specific aspect, the PD-1 ligand binding partners are PDL1 and/or PDL2. In another embodiment, a PDL1 binding antagonist is a molecule that inhibits the binding of PDL1 to its binding partners. In a specific aspect, PDL1 binding partners are PD-1 and/or B7-1. In another embodiment, the PDL2 binding antagonist is a molecule that inhibits the binding of PDL2 to its binding partners. In a specific aspect, a PDL2 binding partner is PD-1. The antagonist may be an antibody, an antigen binding fragment thereof, an immunoadhesin, a fusion protein, or oligopeptide. Exemplary antibodies are described in U.S. Pat. Nos. U.S. Pat. Nos. 8,735,553, 8,354,509, and 8,008,449, all incorporated herein by reference. Other PD-1 axis antagonists for use in the methods provided herein are known in the art such as described in U.S. Patent Application No. US20140294898, US2014022021, and US20110008369, all incorporated herein by reference.

In some embodiments, the PD-1 binding antagonist is an anti-PD-1 antibody (e.g., a human antibody, a humanized antibody, or a chimeric antibody). In some embodiments, the anti-PD-1 antibody is selected from the group consisting of nivolumab, pembrolizumab, and CT-011. In some embodiments, the PD-1 binding antagonist is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PD-1 binding antagonist is AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. CT-011, also known as hBAT or hBAT-1, is an anti-PD-1 antibody described in WO2009/101611. AMP-224, also known as B7-DCIg, is a PDL2-Fc fusion soluble receptor described in WO2010/027827 and WO2011/066342.

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to CD80 or CD86 on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to CD80 and CD86, also called B7-1 and B7-2 respectively, on antigen-presenting cells. CTLA4 transmits an inhibitory signal to T cells, whereas CD28 transmits a stimulatory signal. Intracellular CTLA4 is also found in regulatory T cells and may be important to their function. T cell activation through the T cell receptor and CD28 leads to increased expression of CTLA-4, an inhibitory receptor for B7 molecules.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al. (1998) Proc Natl Acad Sci USA 95(17): 10067-10071; Camacho et al. (2004) J Clin Oncology 22(145): Abstract No. 2505 (antibody CP-675206); and Mokyr et al. (1998) Cancer Res 58:5301-5304 can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001014424, WO2000037504, and U.S. Pat. No. 8,017,114; all incorporated herein by reference.

An exemplary anti-CTLA-4 antibody is ipilimumab (also known as 10D1, MDX-010, MDX-101, and Yervoy®) or antigen binding fragments and variants thereof (see, e.g., WO 01/14424). In other embodiments, the antibody comprises the heavy and light chain CDRs or VRs of ipilimumab. Accordingly, in one embodiment, the antibody comprises the CDR1, CDR2, and CDR3 domains of the VH region of ipilimumab, and the CDR1, CDR2 and CDR3 domains of the VL region of ipilimumab. In another embodiment, the antibody competes for binding with and/or binds to the same epitope on CTLA-4 as the above-mentioned antibodies. In another embodiment, the antibody has at least about 90% variable region amino acid sequence identity with the above-mentioned antibodies (e.g., at least about 90%, 95%, or 99% variable region identity with ipilimumab).

Other molecules for modulating CTLA-4 include CTLA-4 ligands and receptors such as described in U.S. Pat. Nos. U.S. Pat. Nos. 5,844,905, 5,885,796 and International Patent Application Nos. WO1995001994 and WO1998042752; all incorporated herein by reference, and immunoadhesins such as described in U.S. Pat. No. 8,329,867, incorporated herein by reference.

4. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

5. Other Agents

It is contemplated that other agents may be used in combination with certain aspects of the present embodiments to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present embodiments to improve the treatment efficacy.

### I. Kits of the Disclosure

Any of the compositions described herein may be comprised in a kit. In a non-limiting example, cells, reagents to produce cells, vectors, and reagents to produce vectors and/or components thereof may be comprised in a kit. In certain embodiments, NK cells may be comprised in a kit. Such a kit may or may not have one or more reagents for manipulation of cells. Such reagents include small molecules, proteins, nucleic acids, antibodies, buffers, primers, nucleotides, salts, and/or a combination thereof, for example. Nucleotides that encode one or more BCMA-targeting CARs, suicide gene products, and/or cytokines may be included in the kit. Proteins, such as cytokines or antibodies, including monoclonal antibodies, may be included in the kit. Nucleotides that encode components of engineered CAR receptors may be included in the kit, including reagents to generate same.

In particular aspects, the kit comprises the NK cell therapy of the disclosure and also another cancer therapy. In some cases, the kit, in addition to the cell therapy embodiments, also includes a second cancer therapy, such as chemotherapy, hormone therapy, and/or immunotherapy, for example. The kit(s) may be tailored to a particular cancer for an individual and comprise respective second cancer therapies for the individual.

The kits may comprise suitably aliquoted compositions of the present disclosure. The components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also may generally contain a second, third or other additional container into which the additional components may be separately placed. However, various combinations of components may be comprised in a vial. The kits of the present invention also will typically include a means for containing the composition and any other reagent containers in close confinement for commercial sale. Such containers may include injection or blow-molded plastic containers into which the desired vials are retained.

### VII. EXAMPLES

The following examples are included to demonstrate certain non-limiting aspects of the disclosure. It should be appreciated by those of skill in the art that the techniques disclosed in the examples that follow represent techniques discovered by the inventors to function well in the practice of the disclosed subject matter. However, those of skill in the art should, in light of the present disclosure, appreciate that many changes can be made in the specific embodiments that are disclosed and still obtain a like or similar result without departing from the spirit and scope of the disclosed subject matter.

### Example 1

### BCMA-Targeting Chimeric Antigen Receptor in NK Cells

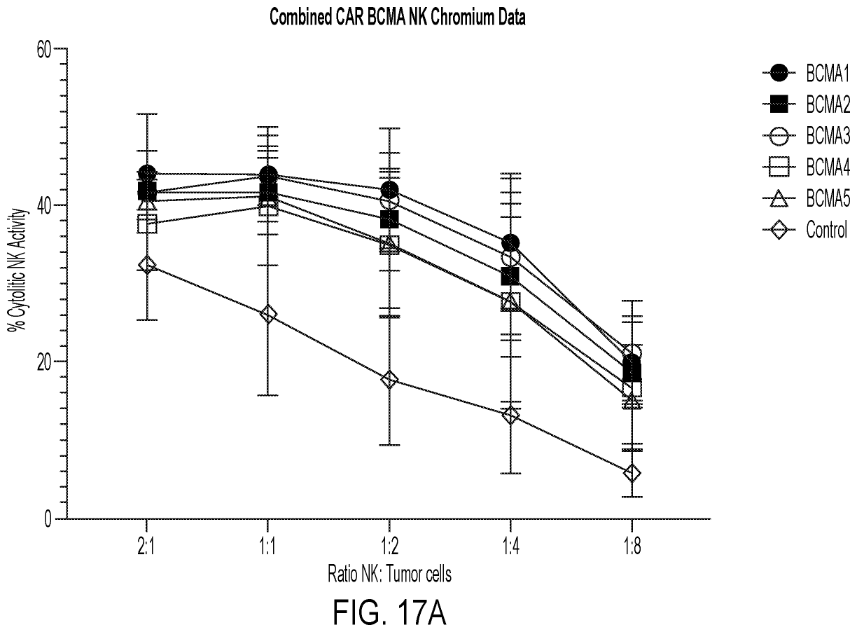

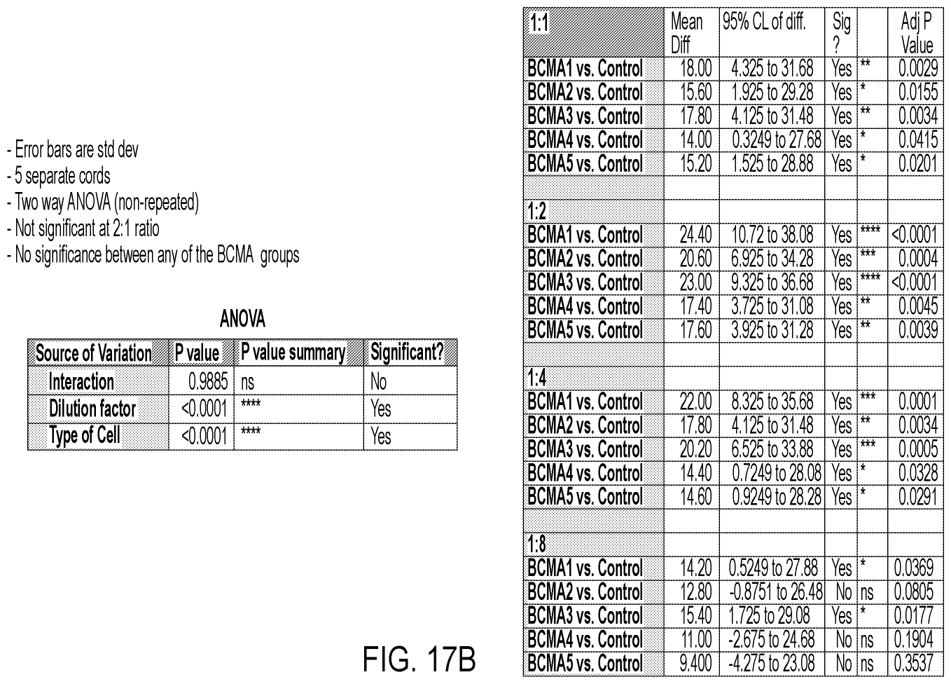

Cord blood-derived NK cells were transduced with one of each of the CAR BCMA constructs (BCMA1-BCMA5) and their cytotoxicity was tested against MM1.S myeloma targets. All 5 constructs were equally effective at increasing the cytotoxicity of NK cells against MM1.S targets compared to non-transduced ex vivo expanded NK cells (FIGS. 17A and 17B). The assay was performed using a standard ⁵¹Chromium assay.

### Example 2

### BCMA-Targeting Chimeric Antigen Receptor in T Cells

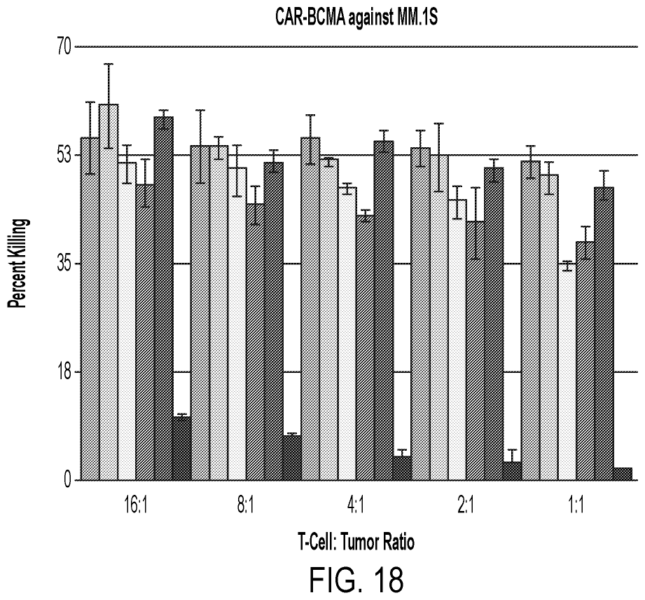

T cells were transduced with each of the BCMA CAR constructs 1-5 and their cytotoxicity was tested against MM1.S myeloma targets. T cells harboring each of the BCMA CAR constructs exert superior cytotoxicity against MM1.S targets compared to non-transduced expanded T cells (FIG. 18). The assay was performed using a standard ⁵¹Chromium assay.

### Example 3

### BCMA-Targeting Chimeric Antigen Receptor in NK Cells

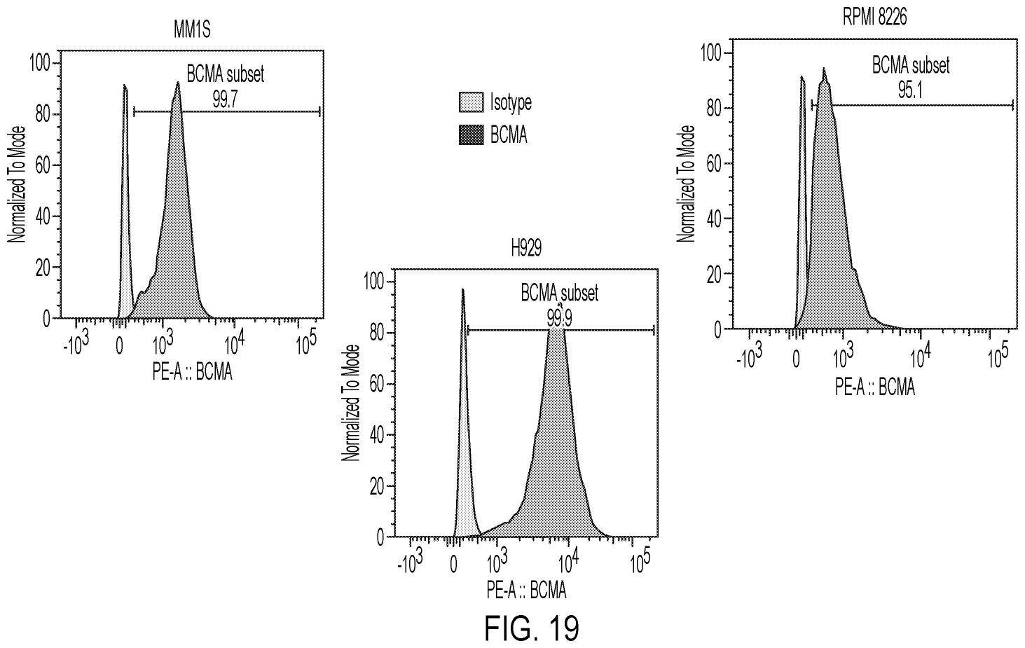

The present example concerns characterization and activity of NK cells bearing BCMA-targeting CAR molecules. As part of the studies, it was shown that multiple myeloma cell lines have surface expression of BCMA (FIG. 19).

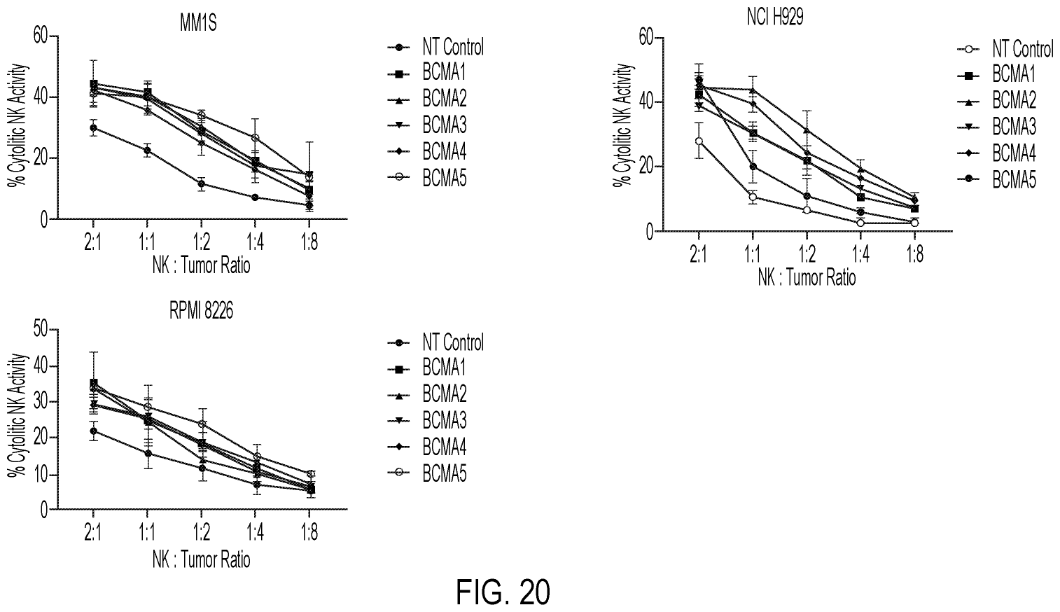

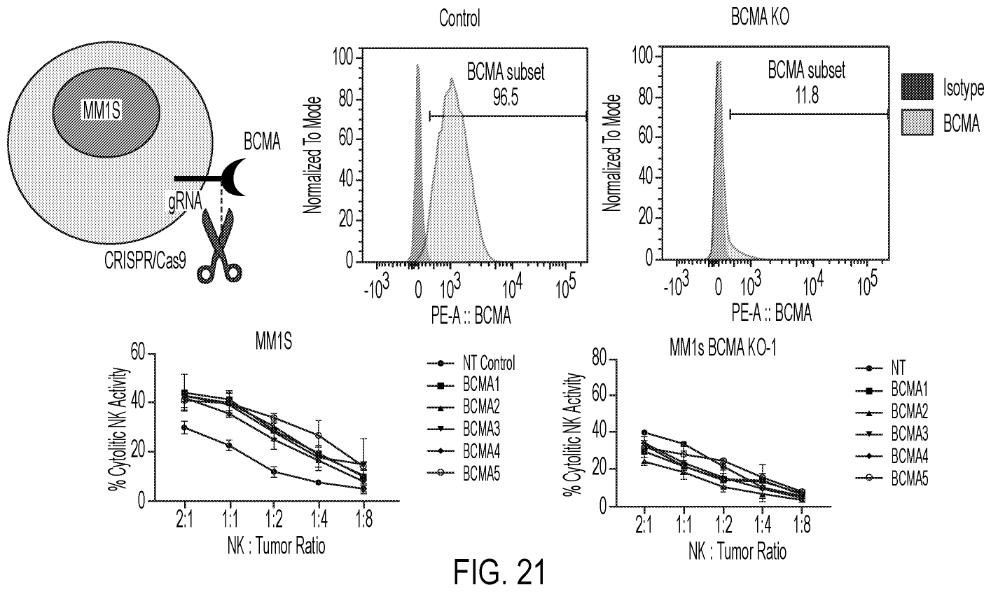

Superior in vitro cytotoxicity was observed by a chromium assay for all BCMA CAR NK cells against MM1S, H929 and RPMI 8226 compared to control NT NK cells. The constructs utilized therein are as follows: BCMA1 is IgSPCOA7D12VLVH28Z15; BCMA2 is CD8SPC11D53VLVH15; BCMA3 is COGSPC11D53VLVHZIL15; BCMA4 is IgSPA7D12VHVL28Z15; and BCMA5 is IgSPA7D12VLVH28Z15 (FIG. 20). FIG. 21 demonstrates that silencing of BCMA by CRISPR deletion in MM1S cell line eliminates enhanced killing from CAR BCMA NK cells, indicating that the killing by the CAR BCMA NK cells is specific.

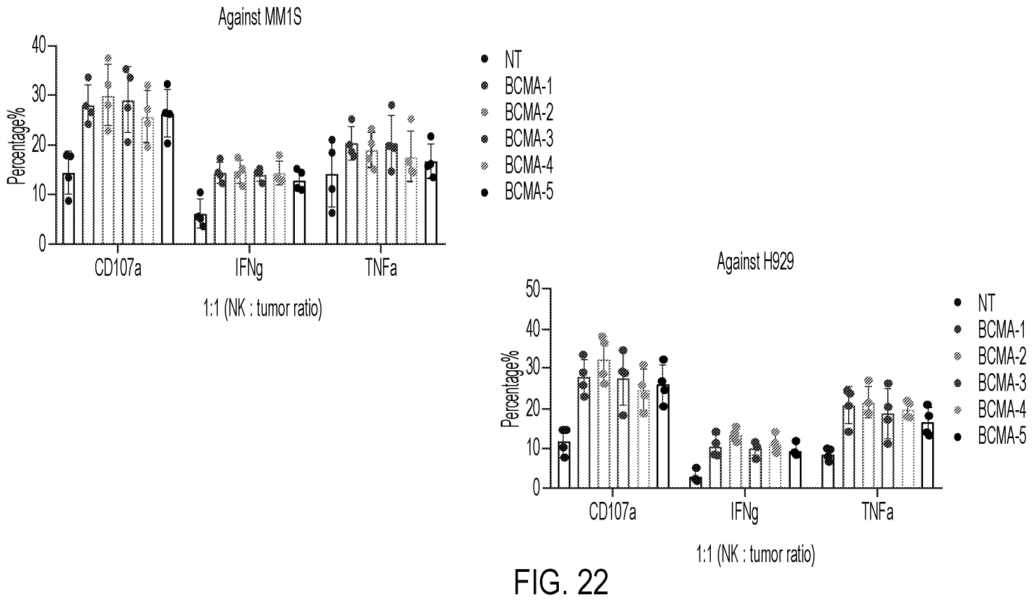

As shown in FIG. 22, BCMA CAR NK cells showed greater degranulation (as represented by CD107a) and produced higher amounts of IFN-γ and TNF-α against MM1S and H929 tumor cells compared to control NT NK cells.

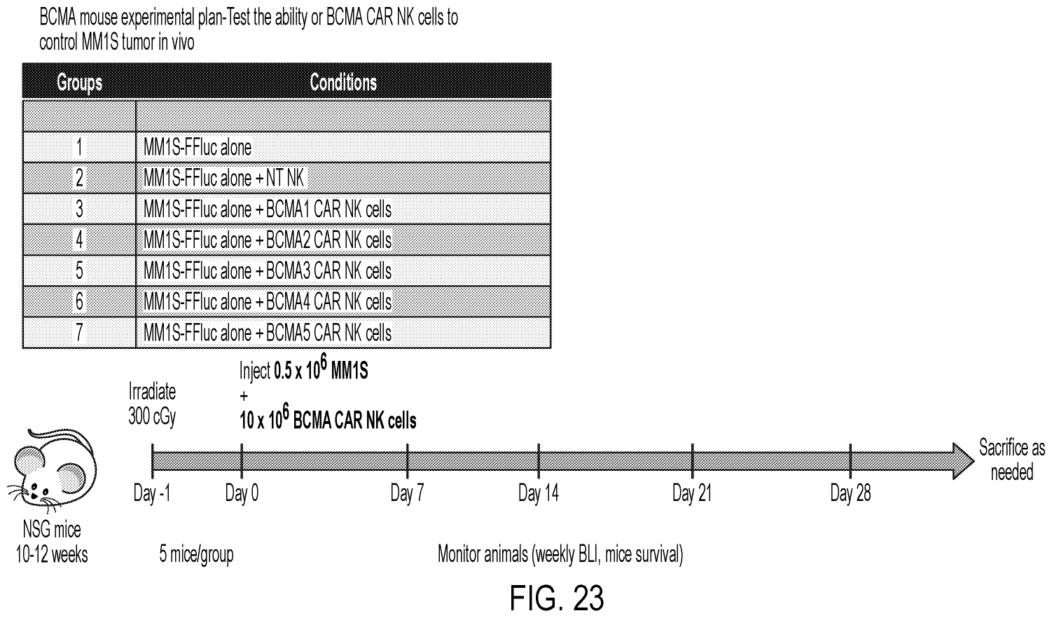

FIG. 23 illustrates an example of an in vivo study to characterize the ability of BCMA CAR NK cells to impact the survival of MM1S tumor-bearing mice.

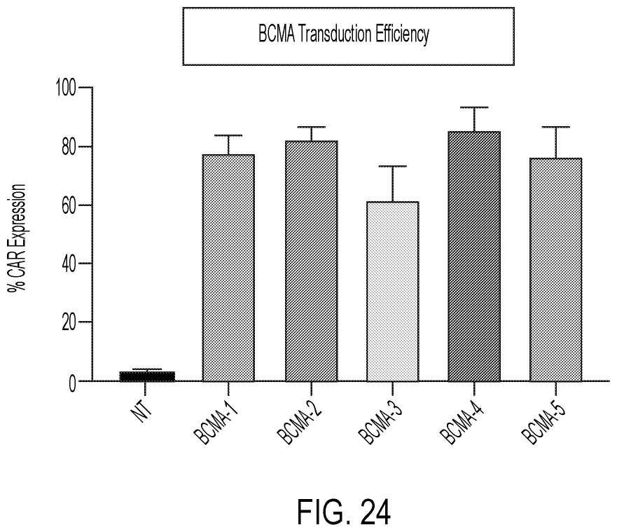

FIG. 24 characterizes the transduction efficiency of NK cells with various BCMA CAR constructs.

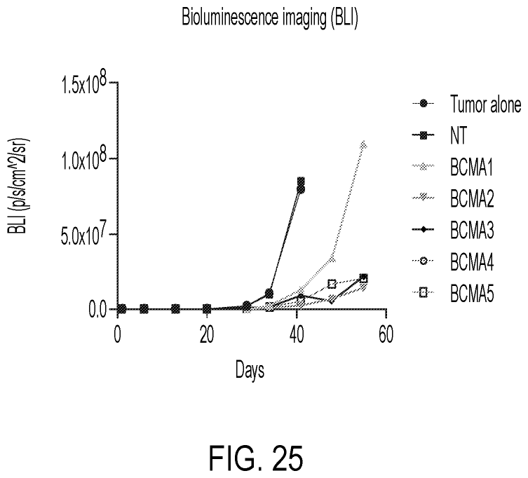

In a MM1S mouse model, BCMA CAR NK cell antitumor activity was assessed, and lower tumor burden was observed for all animals treated with BCMA CAR NK cells when compared to tumor alone or NT NK cells (FIG. 25)

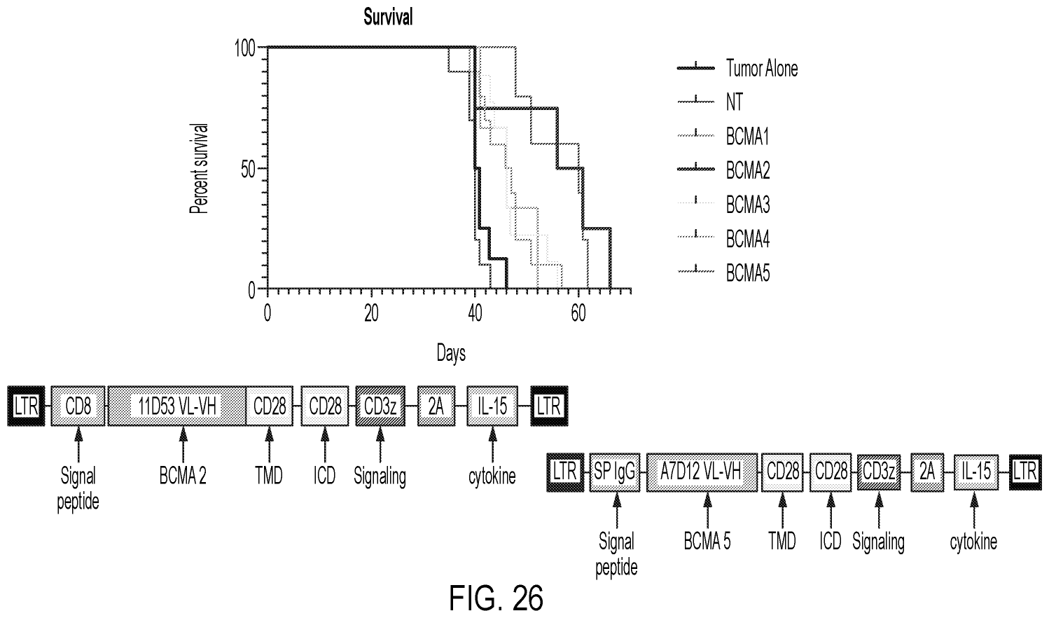

In FIG. 26, the antitumor activity of BCMA CAR NK cells was assessed in a MM1S mouse model. Prolonged survival was observed for all animals treated with BCMA CAR NK cells when compared to tumor alone or NT NK cells. In this study, the BCMA2 (C11D5.3 scFv; VL-VH) and BCMA5 (A7D12; VL-VH) constructs resulted in greater survival than the other CAR constructs.

The present examples show that BCMA CAR NK cells had superior cytotoxicity against multiple myeloma targets (MM1S and NIH929) compared to NT NK cells and that BCMA CAR NK cells exert enhanced antitumor activity and prolonged survival in vivo.

Although the present disclosure and its advantages have been described in detail, it should be understood that various changes, substitutions and alterations can be made herein without departing from the spirit and scope of the design as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

What is claimed is:

1. An expression construct comprising sequence that encodes a B-cell maturation antigen (BCMA)-targeting chimeric antigen receptor (CAR) and that encodes one or both of the following:
(a) a suicide gene; and
(b) a cytokine.

2. The expression construct of claim 1, wherein the CAR comprises a signaling peptide.

3. The expression construct of claim 2, wherein the signaling peptide is from CD8alpha, Ig heavy chain, granulocyte-macrophage colony-stimulating factor receptor, or a signal peptide derived from one or more other surface receptors.

4. The expression construct of any one of claims 1-3, wherein the BCMA-targeting CAR comprises a scFv having a heavy chain and a light chain, and wherein the heavy chain in the sequence that encodes the CAR is upstream of the light chain in a 5′ to 3′ direction.

5. The expression construct of any one of claim 1-3, wherein the BCMA-targeting CAR comprises a scFv having a heavy chain and a light chain, and wherein the heavy chain in the sequence that encodes the CAR is downstream of the light chain in a 5′ to 3′ direction.

6. The expression construct of any one of claims 1-5, wherein the BCMA-targeting CAR comprises a codon optimized scFv.

7. The expression construct of any one of claims 1-6, wherein the BCMA-targeting CAR comprises C11D5.3 scFv, A7D12.2 scFv, CA12A3.2 scFv, C13F12.1 scFv, humanized C11D5.3 scFv, humanized A7D12.2 scFv, humanized CA12A3.2, or humanized C13F12.1 scFv.

8. The expression construct of any one of claims 1-7, wherein the BCMA-targeting CAR comprises one or more costimulatory domains.

9. The expression construct of claim 8, wherein the costimulatory domain is selected from the group consisting of CD28, CD27, OX-40 (CD134), DAP10, DAP12, 4-1BB (CD137), CD40L, 2B4, DNAM, CS1, CD48, NKG2D, NKp30, NKp44, NKp46, NKp80, and a combination thereof.

10. The expression construct of any one of claims 1-9, wherein the CAR comprises CD3zeta.

11. The expression construct of any one of claims 1-10, wherein the CAR comprises a hinge between the scFv and a transmembrane domain.

12. The expression construct of claim 11, wherein the hinge is CD8-alpha hinge, CD28 hinge, the hinge comprises an artificial spacer comprised of Gly3, or the hinge comprises CH1, CH2, and/or CH3 domains of IgGs.

13. The expression construct of any one of claims 1-12, wherein the cytokine is IL-15, IL-12, IL-2, IL-18, IL-21, or a combination thereof.

14. The expression construct of any one of claims 1-13, wherein the suicide gene is a mutant TNF-alpha, inducible caspase 9, HSV-thymidine kinase, CD19, CD20, CD52, or EGFRv3.

15. The expression construct of claim 14, wherein the suicide gene is a mutant TNF-alpha.

16. The expression construct of claim 14 or 15, wherein the mutant TNF-alpha is an engineered nonsecretable mutant TNF-alpha.

17. The expression construct of claim 14, 15, or 16, wherein the TNF-alpha mutant comprises a deletion of the following:
amino acid residue 1 and amino acid residue 12;
amino acid residue 1 and amino acid residue 13;
amino acid residues 1-12;
amino acid residues 1-13; or
amino acid residues −1 to 13.

18. An immune cell, comprising the expression construct of any one of claims 1-17.

19. The immune cell of claim 18, wherein the immune cell is a natural killer (NK) cell, T cell, gamma-delta T cells, macrophages, or invariant NKT (iNKT) cell.

20. The immune cell of claim 18 or 19, wherein the immune cell is a NK cell.

21. The immune cell of claim 20, wherein the NK cell is derived from cord blood, peripheral blood, induced pluripotent stem cells, hematopoietic stem cells, bone marrow, or from a cell line.

22. The immune cell of claim 21, wherein the NK cell line is NK-92 cell line or another NK cell line derived from a tumor or from a healthy NK cell or a progenitor cell.

23. The immune cell of any one of claims 19-22, wherein the NK cell is a cord blood mononuclear cell.

24. The immune cell of any one of claims 19-23, wherein the NK cell is a CD56+NK cell.

25. The immune cell of any one of claims 19-24, wherein the NK cell was expanded in the presence of an effective amount of universal antigen presenting cells (UAPCs).

26. The immune cell of claim 25, wherein the NK cells were cultured with the UAPCs at a ratio of 10:1 to 1:10.

27. The immune cell of claim 25 or 26, wherein the NK cells were cultured with the UAPCs at a ratio of 1:2.

28. The immune cell of any one of claims 25-27, wherein the NK cells were expanded in the presence of IL-2.

29. The immune cell of claim 28, wherein the IL-2 is present at a concentration of 10-500 U/mL.

30. The immune cell of any one of claims 19-29, wherein the NK cells express one or more exogenously provided cytokines.

31. The immune cell of claim 30, wherein the cytokine is IL-15, IL-2, IL-12, IL-18, IL-21, or a combination thereof.

32. A plurality of immune cells of any one of claims 18-31, said cells present in a suitable medium.

33. The plurality of claim 32, wherein the immune cells are NK cells.

34. A method of treating a BCMA-positive cancer in an individual, comprising the step of administering to the individual an effective amount of cells harboring the expression vector of any one of claims 1-17.

35. The method of claim 34, wherein the cells are NK cells, T cells, or iNKT cells.

36. The method of claim 35, wherein the NK cells are derived from cord blood, peripheral blood, induced pluripotent stem cells, bone marrow, or from a cell line.

37. The method of claim 36, wherein the cell line is NK-92 cell line or another NK cell line derived from a tumor or from a healthy NK cell or a progenitor cell.

38. The method of any one of claims 35-37, wherein the NK cells are derived from cord blood mononuclear cells.

39. The method of any one of claims 35-38, wherein the NK cells are CD56+NK cells.

40. The method of any one of claims 35-39, wherein the NK cells were expanded in the presence of an effective amount of universal antigen presenting cells (UAPCs).

41. The method of claim 40, wherein the NK cells were cultured with the UAPCs at a ratio of 10:1 to 1:10.

42. The method of claim 40 or 41, wherein the NK cells were cultured with the UAPCs at a ratio of 1:2.

43. The method of any one of claims 35-42, wherein the NK cells were expanded in the presence of IL-2.

44. The method of claim 43, wherein the IL-2 is present at a concentration of 10-500 U/mL.

45. The method of any one of claims 34-44, wherein the individual has a B cell malignancy, multiple myeloma, lung cancer, breast cancer, thyroid cancer, head and neck cancer, or a combination thereof.

46. The method of any one of claims 34-45, wherein the cells are allogeneic with respect to the individual.

47. The method of any one of claims 34-45, wherein the cells are autologous with respect to the individual.

48. The method of any one of claims 34-47, wherein the individual is a human.

49. The method of any one of claims 34-48, wherein the cells are administered to the individual once or more than once.

50. The method of claim 49, wherein the duration of time between administration of the cells to the individual is hours, days, weeks, or months.

51. The method of any one of claims 34-50, further comprising the step of providing to the individual an effective amount of an additional therapy.

52. The method of claim 51, wherein the additional therapy comprises surgery, radiation, gene therapy, immunotherapy, or hormone therapy.

53. The method of claim 51 or 52, wherein the additional therapy comprises one or more antibodies.

54. The method of any one of claims 34-53, wherein the cells are administered to the individual by injection, intravenously, intraarterially, intraperitoneally, intratracheally, intratumorally, intramuscularly, endoscopically, intralesionally, percutaneously, subcutaneously, regionally, by perfusion, in a tumor microenvironment, or a combination thereof.

55. The method of any one of claims 34-54, further comprising the step of identifying BCMA-positive cells in the individual.

56. The method of claim 55, wherein the identifying step utilizes antibodies.

57. As a composition of matter, the sequences of SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:151, SEQ ID NO:152, SEQ ID NO:153, SEQ ID NO:154, SEQ ID NO:155, SEQ ID NO:156, SEQ ID NO:157, or SEQ ID NO:158.
